(19) United States
(12) Patent Application Publication
(10) Pub. No.: US 2015/0212068 A1
(43) Pub. Date: Jul. 30, 2015

(54) Cholyl-L-Lysyl-Fluorescein Assay

(71) Applicant: Novassay SA, Yverdons-les-Bains (CH)

(72) Inventor: Marc Jonathan Kearsey, Paris (FR)

(73) Assignee: Novassay SA, Yverdon-les-Bains (CH)

(21) Appl. No.: 14/423,094

(22) PCT Filed: Aug. 20, 2013
(86) PCT No.: PCT/EP2013/067280

§ 371 Date: Feb. 20, 2015

Related U.S. Application Data
(60) Provisional application No. 61/691,089, filed on Aug. 20, 2012.

(51) Int. Cl. G01N 33/50; G01N 33/58; G01N 33/72

(52) U.S. Cl. CPC G01N 33/5014; G01N 33/728; G01N 33/582; G01N 2800/085

(57) ABSTRACT

Fluorescent bile salt derivatives such as cholyl-L-lysyl-fluorescein have shown diagnostic potential to evaluate biliary elimination. The invention relates to the development of a novel competitive Enzyme-linked immunosorbent assay (ELISA) assay to quantify fluorescent bile salt derivatives for the evaluation of drug-drug interactions and for diagnostic applications.

The present invention relates to Cholyl-L-Lysyl-Fluorescein (CLF) assays, and particularly, although not exclusively, to quantitative CLF assays to measure CLF levels in biological fluids. The invention extends to the use of such assays in a range of different biological applications, for example fluorescent bile salt detection, monitoring pharmacokinetic parameters (distribution and elimination) following IV administration of CLF, in order to provide an in vivo diagnostic test primarily to monitor drug-drug interactions, characterization of drug-drug interactions in in vitro cellular assays, and a range of other diagnostic purposes.

Bile acids have been implicated in the pathogenesis of several diseases primarily affecting the liver. A methodology has been developed to measure the levels of bile acids (in vitro) in plasma samples through the addition of a known quantity of a tagged derivative of said bile acid followed by capture of both the bile acid and tagged derivative using a limited quantity of antibody specific to the bile acid. The amount of tagged bile acid captured is inversely proportional to the amount of free bile acid in the plasma sample. This methodology can be quantifiable and has been used to determine whether a liver is diseased or damaged, and for determining the presence, in humans, of hepatobiliary diseases including viral hepatitis, hepatic malignancy, cholestasis, hepatic cirrhosis, and biliary atresia (Hixson et al. U.S. Pat. No. 4,264,514).

Drug-induced cholestasis, the pathophysiological syndrome resulting from impaired hepatic bile flow, is a significant mechanism of adverse drug reactions, and the inhibition of bile acid efflux transporters is believed to be one mechanism of drug-induced cholestasis. With reference to FIG. 32, Cholyl-L-Lysyl-Fluorescein (CLF) is a fluorescein-labelled bile acid whose biological behaviour closely resembles cholyl glycine, and is taken up by the OATP1B3 and ABCC2/Abcc2 transporters which most likely mediate hepatic uptake and biliary excretion of CLF, respectively. As such, CLF has been used in a commercialized test (GENTEST®, Becton Dickinson, Bedford, Mass., USA) as an agent to visualize the bile canaliculi and also to assess cholestatic hepatotoxicity in human and rodent primary hepatocyte cultures. This methodology is able to assess in vitro hepatic cholestasis in the presence of a cholestatic agent or a drug.

Drug interactions occur when one drug alters the effect of another drug, and when both drugs concurrently reside in the body (drug-drug interaction). This action can be additive or synergistic (when the drug's effect is increased), but can also be antagonistic (when the drug's effect is decreased) or a new effect can be produced that neither produces on its own.

Such interactions can therefore affect the efficacy of a given drug but can also negatively influence the safety of a given drug and result in adverse or serious adverse events. It is therefore important to evaluate such pharmacological interactions both in vitro and in vivo prior to testing novel drugs in patients, during later stage clinical and post-registration studies. Such drug-drug interactions may affect the excretion/elimination and liver metabolism of certain compounds through altering the efficacy of certain anionic or cationic carriers. In so doing the pharmacokinetics of certain drugs may be significantly modified. In addition, certain disease states and/or certain genetic isoforms of drug carriers may influence drug excretion/elimination and metabolism.

CLF has been used as an agent to determine liver function in patients following an intravenous injection of CLF into the patient and then assessing the colour or fluorescence in plasma samples and fitting the acquired values on a comparative plasma elimination curve for CLF (Mills, U.S. Pat. No. 6,030,841). Such a methodology only provides an indirect quantification of CLF (through fluorescence). Such prior art does not however suggest any utility of CLF to evaluate drug-drug interactions in experimental animals and in human subjects and also for the detection and monitoring of other pathologies.

Novel and sensitive methods are therefore required to evaluate drug-drug interactions, for bile-specific elimination of drugs and also for the detection and monitoring of other pathologies.

As described in Example 4, the inventors set out to develop high performance liquid chromatography (HPLC) tandem mass spectrometry (MS/MS) and ELISA assays for the quantification of Cholyl-Lysyl-L-Fluorescein (CLF) in biological samples, including human blood plasma. In their initial experiments, two working solutions of CLF were prepared, the first was CLF dissolved in 100% methanol and the second was CFL in aqueous ethanol under a basic pH (i.e. water/ethanol/sodium hydroxide). However, the inventors observed surprising differences between the quantification of CLF in the plasma samples using HPLC-MS/MS method and the ELISA methodology. The inventors have unexpectedly found that a bile salt derivative conjugated with CLF is more tautomerically stable when solubilised in an alcohol (such as methanol) as compared with the prior art methods and techniques, wherein the bile salt conjugate is solubilised in the presence of an alkali, such as sodium hydroxide. The inventors therefore consider that compositions comprising a bile salt conjugated with CLF when solubilised in an alcohol (e.g. methanol) is not only more chemically stable, but is also more stable when measured using immunological techniques. The inventors therefore believe that any assay or analysis using the compositions disclosed herein in which alcohol is used will have greater sensitivity than compared with a similar assay having a bile salt solubilised with another composition, for example an acid or an alkali. This discovery could not have been predicted in view of the prior art and therefore the invention comprises unexpectedly superior results.

Thus, in a first aspect of the invention, there is provided a method for preparing a sample for analysis in an assay for Cholyl-L-Lysyl-Fluorescein (CLF), the method comprising contacting a sample comprising CLF with an alcohol under non-basic conditions.

Therefore, the method of the first aspect preferably precedes a CLF assay.

Hence, in a second aspect of the invention, there is provided a Cholyl-L-Lysyl-Fluorescein (CLF) assay for determining the concentration of CLF in a sample, the method comprising—
(i) contacting a sample comprising CLF with an alcohol under non-basic conditions, and
(ii) exposing the resultant sample to a CLF assay, to thereby determine the concentration of the CLF.

Preferably, the method of the first aspect is initially carried out prior to the CLF assay.

The inventors believe that they are the first to have produced a novel composition comprising CLF and an alcohol but under non-basic conditions, i.e. in the absence of alkali.

As such, in a third aspect, there is provided a composition comprising Cholyl-L-Lysyl-Fluorescein (CLF) and an alcohol, wherein the composition does not comprise alkali.

In a fourth aspect, there is provided use of the composition of the third aspect for:—
(i) quantifying Cholyl-L-Lysyl-Fluorescein (CLF) in a sample;
(ii) the in vitro determination of a drug-drug interaction (DDI) in a subject;
(iii) the in vitro detection of cholestatic hepatoxicity in a subject;
(iv) measuring in vitro biliary elimination of a drug from a subject;
(v) diagnosing disease in a subject; or
(vi) a competitive immunoassay for detecting CLF in a sample.

Corresponding methods of each use (i) to (vi) of the fourth aspect are also envisaged and form separate aspects of the invention.

For example, in a fifth aspect, there is provided a method for determining drug-drug interactions (DDI) in a subject comprising:—
(a) administering, into the bloodstream of a subject, Cholyl-L-Lysyl-Fluorescein (CLF);
(b) contacting a first blood sample, taken from the subject at time (T)=0 after the administration of one or more pharmacologically active compound(s), with an alcohol under non-basic conditions, and measuring the CLF concentration in the subject's plasma;
(c) contacting a second blood sample, taken from the subject at T=X, wherein X represents a unit of time after the administration of the one or more pharmacologically active compound(s), with an alcohol under non-basic conditions, and measuring the CLF concentration in the subject's plasma; and
(d) comparing the CLF concentration at T=X with that of T=0, whereby a difference in plasma levels of the CLF is determined, and the DDI is thereby evaluated.

In a sixth aspect, there is provided an analyte or test composition comprising a physiological substrate conjugated to a signalling molecule, wherein the analyte or test composition is soluble in alcohol under non-basic conditions, for use in determining the in vivo serum or plasma levels of a composition whose metabolism is affected by the in vivo liver enzyme activity, wherein the liver enzyme activity corresponds to a drug-drug interaction, detoxification of a drug metabolite, or metabolism of the analyte.

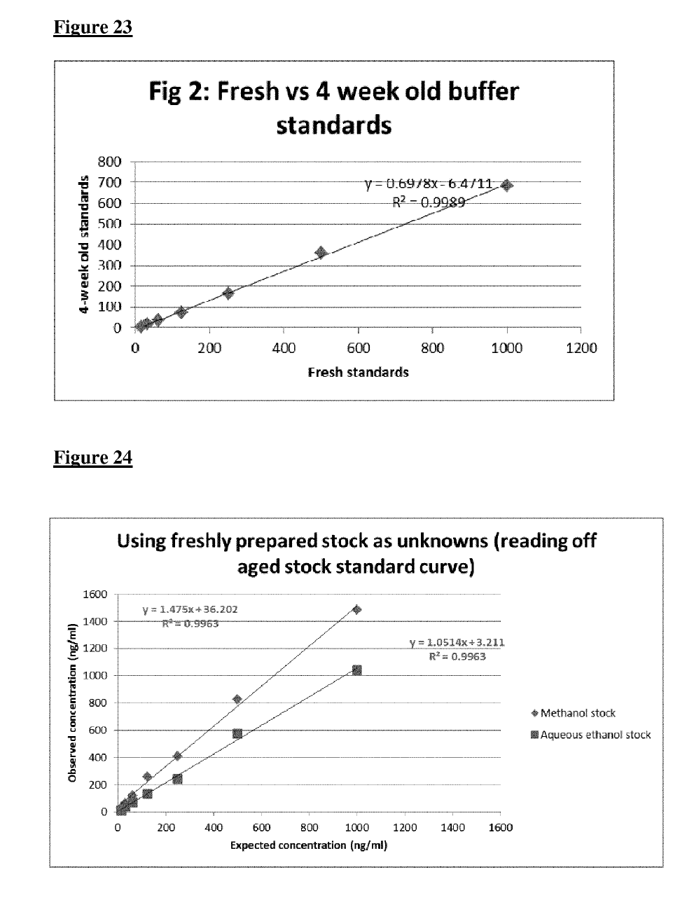

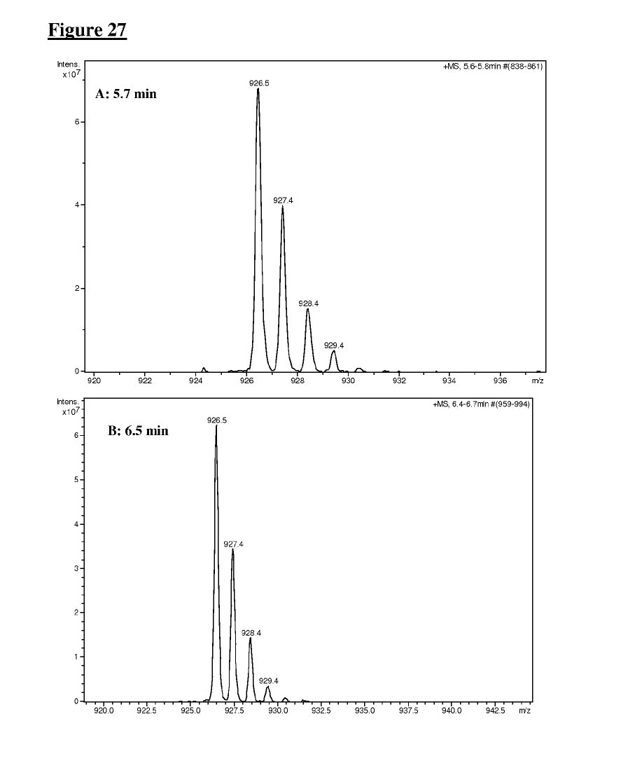

As shown in FIGS. 2 and 5, the selected mass spectrometric conditions led to the production of specific daughter ions from a specific [M+H]⁺ parent ion for CLF, and the retention times for CLF and its internal standard (IS) were 6.5 and 6.9 min, respectively. The UV chromatograms obtained at 254 nm are shown in FIG. 26. In the working solution prepared in methanol (i.e. SS CLF-MetOH) and in a control solution prepared in ammonium bicarbonate (0.1 M), one major chromatographic peak was observed having a retention time of 6.5 min, which corresponded to CLF. However, in the working solution prepared in aqueous ethanol-sodium hydroxide (SS CLF-EtOH), two additional chromatographic peaks were observed at 5.7 and 9.7 min. The inventors found that the peak area of CLF was around 10-fold lower in aqueous ethanol than in methanol and aqueous ammonium bicarbonate. Furthermore, the inventors believed that the two additional peaks from the aqueous sample reflected impurities, and the inventors believed that these were likely due to inherent instability of CLF.

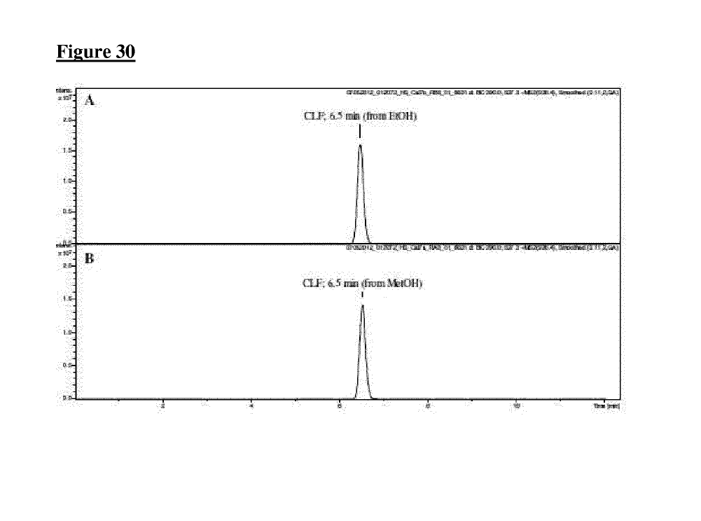

However, as described in Example 4, the compounds that were believed to be impurities were in fact shown to be tautomers of the fluorescein moiety of CLF, as shown in FIG. 29.

This reaction commonly results in the formal migration of a hydrogen atom or proton, accompanied by a switch of a single bond and adjacent double bond. However, because of the rapid inter-conversion, tautomers are generally considered to be the same chemical compound. Surprisingly, the inventors have demonstrated that the tautomerization of CLF, and especially of the fluorescein moiety, is pH-dependant. In other words, CLF is in its native form when the pH of the solution is below [pKa−2]. The addition of sodium hydroxide (i.e. a base) has moved the steady state to the other tautomeric form (i.e. pH>pKa+2). Thus, the inventors have found that the presence of the hydroxyl ions (i.e. a base) pushes the pH equilibrium to the right of the equation, that is, an increased, more alkali, pH, thereby causing increased tautomerization of the compositions in solution into two components, as disclosed in the Examples. However, in the ELISA assay, the sodium hydroxide was diluted and buffered in the plasma and the pH of samples was decreased in order to obtain another steady state of CLF. The isomeric form of CLF was not detected in standard samples as shown in FIG. 30. The working solution was believed to be buffered in the plasma and during the sample extraction, and this observation showed that the tautomerization of CLF is reversible and depends on the pH of the medium. Therefore, in the ELISA assay, CLF was quantified in its native form. Furthermore, the fluorescence properties of both isomeric forms are also likely to be different. Indeed, quantum yield of fluorescence depends of the time-life of the excited state of the fluorophore (as shown by the colour of working solutions). CLF was in its native form in the ammonium bicarbonate solution.

The prior art compositions comprised increased tautomerization of the bile salt derivative conjugated with the fluorescein molecule, thereby resulting in a reduced number of native bile salt conjugated with fluorescein molecules potentially available for detection for example by immunoassay (for example, ELISA), HPLC, or other methods, as disclosed below. The improved methods, assays and compositions of the invention have utility since the resulting calibration standard curves that are determined have improved sensitivity that those of the prior art and in current use. The resulting new standard curves will enable clinicians, pharmacologists, and related clinical professionals to be able to better determine blood plasma levels of CLF, and thereby be able to determine improved prognoses, outcomes, and the like. It is evident to one of skill in the art that if a calibration standard curve using incremental quantities of a particular composition give a lower measurement (for example, optical density or peak area value), then test samples comprising the same composition will be determined to comprise lower levels of said composition than are actually present. In addition, at potential saturating values, the sensitivity of the prior art assay may be reduced. Therefore, the compositions herein disclosed and described will enable one of skill in the art to form a more complete understanding of CLF levels in a sample.

In view of the above, advantageously, by contacting the sample with an alcohol under non-basic conditions in the methods, uses and composition of the invention, it is preferred that the CLF is retained substantially in its non-tautomered conformation. It will be appreciated that the non-tautomered conformation of CLF is as represented on the left hand side of FIG. 29. As shown in FIG. 26, the retention time for the non-tautomered conformation of CLF is about 6.5 min (see the UV chromatograms obtained at 254 nm), which is clearly distinguishable from CLF tautomers having a retention time of 5.7 min.

In one embodiment, the sample in the methods and uses of the invention may be a test sample, for example a biological sample obtained from the subject. The sample may be a blood sample, which may be either serum or plasma. Preferably, the sample is blood plasma. In another embodiment, the sample may be a control sample, for example one which is used to form a standard curve and against which an unknown test sample may be measured.

The alcohol contacted with the sample may comprise an aliphatic alcohol, preferably a C1-C5 alcohol. In one embodiment, the alcohol is selected from the group consisting of: methanol, ethanol and propanol. In a preferred embodiment, the alcohol is selected from the group consisting of methanol and ethanol. In a most preferred embodiment, the alcohol is methanol.

Preferably, the concentration of alcohol (which is preferably, methanol) following contacting with the sample is at least 30% (v/v), more preferably at least 40% (v/v), even more preferably at least 50% (v/v). It is preferred that the concentration of alcohol following contacting with the sample is at least 60% (v/v), and most preferably at least 70% (v/v).

Preferably, the sample is contacted with an alcohol (which is preferably methanol) under non-basic conditions. Thus, the pH is preferably 7.0 or less. The pKa of the OH group in methanol is about 15, and about 16 in ethanol. As such, there is little or no ionisation under normal conditions and any pH measurements of an alcohol solvent will be about 7.0. Hence, the pH of the resultant sample may be neutral or acidic. Preferably, the sample is contacted with the alcohol in the absence of alkali, and preferably in the absence of sodium hydroxide. The uses and methods of the invention preferably comprise quantifying the concentration of Cholyl-L-Lysyl-Fluorescein (CLF) in the sample using an immunoassay, for example an enzyme-linked immuno-specific assay (ELISA), and preferably a sandwich ELISA format. The uses and methods of the invention preferably comprise the use of a competitive immunoassay for the detection of the CLF in the sample. It will be appreciated that a sandwich ELISA involves using two different antibodies that recognise different epitopes on the same target molecule, a capture and a detecting antibody, in order to have a quantitative test with sufficient sensitivity.

Preferably, the uses and methods of the invention comprise measuring plasma levels of CLF using a fluorescein-specific monoclonal antibody. Preferably, the method comprises determining the levels of CLF by measuring the signal generated by a coupled secondary antibody using enzyme-linked immuno-specific assay (ELISA) based on a known standard curve. The standard curve may be generated using the method of the first aspect.

Preferably, the method comprises determining the levels of CLF by measuring the signal generated by a competitive enzyme-linked immuno-specific assay (ELISA). In a preferred embodiment, the measurement of dynamic elimination kinetics between T=X and T=0, following injection of CLF, provides specific disease-characteristic signatures. In a preferred embodiment, the method can be used to provide a single diagnostic test capable of identifying and differentiating pathologies affecting the following organs/tissues: liver, kidneys, intestine, blood-brain barrier, blood-testicular barrier, placenta, and single or multi-organ drug-drug interactions. Accordingly, diseases which may be diagnosed by the uses and methods of the invention are preferably selected from a disease affecting the following organs/tissues: liver, kidneys, intestine, blood-brain barrier, blood-testicular barrier and placenta.

As described in the Examples, the inventors have developed two independent competitive immunoassay formats in accordance with the invention. In one preferred embodiment (Assay format 1), the immunoassay preferably comprises a biotin conjugated anti-FITC (fluorescein isothiocyanate) antibody (which may be either monoclonal or polyclonal), a FITC-Horse Radish Peroxidase (HRP) conjugate, CLF (fluorescein (FITC)-conjugated bile salt) and a streptavidin-coated ELISA microtitre plate. The biotinalyated antibody is preferably captured by the streptavidin-coated ELISA plate, and competition for antibody binding occurs between fluorescein (FITC)-conjugated bile salt and the FITC-HRP conjugate. The amount of FITC-HRP may then be amplified and quantified directly through an enzymatic colorimetric reaction, for example using tetramethylbenzidine (TMB) substrate.

In another preferred embodiment (Assay format 2), the anti-FITC antibody is preferably conjugated to HRP (anti-FITC-HRP) and the FITC is preferably conjugated to biotin (FITC-biotin). In this assay format, the FITC-biotin conjugates are preferably captured on the biotinylated ELISA plate (rather than the antibody in Assay format 1). The fluorescein (FITC)-conjugated bile salt and FITC-biotin conjugate preferably compete for binding to the anti-FITC-HRP antibody. Unbound antibody is then preferably removed, and the amount of remaining antibody may be quantified colorimetrically, for example using tetramethylbenzidine (TMB) substrate. Standard curves may be established with known concentrations of fluorescein (FITC)-conjugated bile salt, which will compete for FITC-HRP or anti-FITC-HRP binding and allow the quantification of an unknown fluorescein (FITC)-conjugated bile salt in a biological fluid to be defined (see FIG. 4).

Preliminary experimental data quantifying CLF in PBS (phosphate buffered saline) has showed that both Assay format 1 and 2 worked well. However, testing in serum samples showed lower recovery, which suggested significant protein binding and higher than acceptable inter-serum variation. A number of different agents were experimentally tested to establish whether binding of the fluorescein-conjugated bile salt to serum proteins could be blocked, including 8-anilino-1-naphthalene sulfonic acid (ANS, 0.2%), deoxycholic acid, cholic acid, low/high pH, phenol red and Orange G. ANS and deoxycholic acid showed the best blocking characteristics, but the ANS blocking agent showed superior characteristics and was adopted. Both immunoassay formats were then compared using the ANS blocking substrate. Assay format 2 (HRP-anti-FITC antibody) showed superior recovery and accuracy and was therefore adopted and further optimised.

Therefore, preferably the immunoassay comprises the use of a blocking agent to inhibit binding between the fluorescein-conjugated bile salt and serum proteins. Preferably, the blocking agent comprises 8-anilino-1-naphthalene sulfonic acid (ANS) or deoxycholic acid, and most preferably ANS.

In another embodiment, the use of the fourth aspect may comprise quantifying CLF in the sample using HPLC.

In another aspect, the invention contemplates an analyte or a test composition for use in determining the in vivo serum levels of a composition whose metabolism is affected by the in vivo metabolic processes, such as liver enzyme activity, wherein the liver enzyme activity relates to drug-drug interactions, detoxification of drug metabolites, metabolism of the analyte, ADME, and the like.

In one embodiment the analyte or test composition comprises a physiological substrate conjugated to a signalling molecule, wherein the analyte is soluble in alcohol. In one embodiment the signalling molecule is a fluorescent composition. In another embodiment, the signalling molecule is a radiological composition. In another embodiment, the signalling molecule is an immunological composition (for example, biotin, etc.). In a yet other embodiment, the signalling molecule is a colorimetric molecule, a fluorescent compound, anthocyanins, green fluorescent protein (GFP), β-glucuronidase, luciferase, Cy3, Cy5 chemiluminescent, or chromogenic agents.

In one embodiment the analyte or test composition comprises a bile salt, wherein the bile salt is selected from the group of cholic acid derivatives. In a preferred embodiment, the analyte comprises a derivatized bile salt, wherein the derivatizing moiety is a signalling molecule.

In a preferred embodiment, the analyte or test composition comprises the bile salt derivative cholyl-L-lysyl-fluorescein (CLF) and an alcohol. In another embodiment, the analyte comprises cholyl-L-lysyl-fluorescein (CLF) and an alcohol, wherein the CLF is substantially in its native non-tautomered conformation. In a yet other embodiment, the analyte comprises cholyl-L-lysyl-fluorescein (CLF) and an alcohol but not an alkali.

In one embodiment the alcohol is selected from the group consisting of methanol, ethanol, propanol, etc. In a preferred embodiment, the alcohol is selected from the group consisting of methanol and ethanol. In a most preferred embodiment, the alcohol is methanol. In one other preferred embodiment, the alcohol concentration in the analyte is at least 30% v/v. In one other preferred embodiment, the alcohol concentration in the analyte is at least 40% v/v. In one other preferred embodiment, the alcohol concentration in the analyte is at least 50% v/v. In another more preferred embodiment, the alcohol concentration in the analyte is at least 60% v/v. In a yet more preferred embodiment, the alcohol concentration in the analyte is at least 70% v/v.

In another embodiment, the invention contemplates a test composition, the test composition comprising cholyl-L-lysyl-fluorescein (CLF) and an alcohol.

In another embodiment, the invention contemplates a test composition, the test composition comprising cholyl-L-lysyl-fluorescein (CLF) and an alcohol, wherein the CLF is substantially in its native non-tautomered conformation.

In another embodiment, the invention contemplates a test composition, the test composition comprising cholyl-L-lysyl-fluorescein (CLF) and an alcohol but not an alkali. In a preferred embodiment, the alcohol is selected from the group consisting of methanol, ethanol, propanol, isopropanol.

In another preferred embodiment the analyte comprises cholyl-L-lysyl-fluorescein (CLF) and ammonium bicarbonate. In another preferred embodiment the test composition comprises cholyl-L-lysyl-fluorescein (CLF) and ammonium bicarbonate.

In another embodiment the invention contemplates a method for determining drug-drug interactions (DDI) in an experimental animal or human subject, the method comprising determining the plasma levels of a cholyl-L-lysyl-fluorescein (CLF) in the experimental animal or the human subject, the method comprising the steps of (a) administering into the bloodstream of the experimental animal or the human subject a compound comprising CLF as disclosed herein, (b) measuring the CLF concentration in the animal's or subject's plasma at time (T)=0 after the administration of a pharmacologically active compound or a combination of two or more pharmacologically active compounds, (c) measuring the CLF concentration in the animal's or subject's plasma at T=X, wherein X represents a unit of time after the administration of a pharmacologically active compound or a combination of two or more pharmacologically active compounds, (d) comparing the CLF concentration at T=X with that of T=0, whereby the difference in plasma levels of the CLF is determined and the DDI is thereby evaluated.

In a preferred embodiment, the method comprises the step of further measuring the CLF concentration in plasma of an experimental animal or human subject at a number of different time points thereby creating a dynamic pattern of measurement. In a preferred embodiment, a specific dynamic measurement is characteristic of a specific disease state. In a preferred embodiment, the disease state is selected from a disease affecting the following organs/tissues: Liver, Kidneys, Intestine, Blood-Brain Barrier, Blood-Testicular Barrier and Placenta.

In a more preferred embodiment, the specific dynamic pattern is characteristic of multi-organ drug-drug interactions. In a preferred embodiment, the method measures plasma levels of CLF using a fluorescein-specific monoclonal antibody.

In a preferred embodiment, the method determines the levels of CLF by measuring the signal generated by a coupled secondary antibody using enzyme-linked immuno-specific assay (ELISA) based on a known standard curve. In a preferred embodiment, the method determines the levels of CLF by measuring the signal generated by a competitive enzyme-linked immuno-specific assay (ELISA). In a preferred embodiment, the measurement of dynamic elimination kinetics following injection of CLF provides specific disease-characteristic signatures.

In a preferred embodiment, the method can be used to provide a single diagnostic test capable of identifying and differentiating pathologies affecting the following organs/tissues: Liver, Kidneys, Intestine, Blood-Brain Barrier, Blood-Testicular Barrier, Placenta, and Multi-organ drug-drug interactions.

In another more preferred embodiment, the invention contemplates a composition comprising X-linker-Y, where X is any bile salt or other circulation hepatic transported molecule and Y can be FITC or any other structure (or tag) that can be captured by a monoclonal antibody and where Y does not significantly alter the elimination of X. linker is a stable connection between X and Y that does not undergo hepatic metabolism.

### DETAILED DESCRIPTION OF THE INVENTION

This invention relates to the development of a novel competitive immunoassay for the detection of fluorescent conjugated bile salt derivatives and their use for detection of drug-drug interactions. It also relates to the use of the invention as a diagnostic tool for quantification of biliary elimination of drugs, renal, intestinal as well as diseases affecting the placenta and the blood-brain barrier.

There are a number of different potential immunoassay formats and detection strategies that could be adopted for such a test, but it is not possible for someone knowledgeable in the art to determine which format would be appropriate without significant experimental validation. Such an immunoassay would usually be performed using a sandwich ELISA format (using two different antibodies that recognise different epitopes on the same target molecule, a capture and a detecting antibody) in order to have a quantitative test with sufficient sensitivity. The selection of the optimal antibody combination for such a sandwich immunoassay is not however trivial, in the case of fluorescein-conjugated bile salts. The bile portion of the fluorescein-conjugated bile salt can not be used as a capture antibody in biological fluids where the bile salt is present endogenously as it will compete for binding with the fluorescein-conjugated bile salt. In addition, the use of conventional sandwich ELISA methods is not appropriate due to the low molecular size of the fluorescent conjugated bile salt (in the region of 800-1000 MW (Daltons, Da), for which stearic hindrance prevents the use of monoclonal antibodies that recognise two independent epitopes on the fluorescein-conjugated bile salt. The use of a capture antibody specific for the fluorescein and/or linker region of the fluorescein-conjugated bile salt followed by direct detection of the fluorescence is also problematic as the signal generated would not provide an assay with sufficient sensitivity. Novel immunoassay techniques were therefore developed to circumvent these issues and obtain a robust immunoassay with sufficient sensitivity.

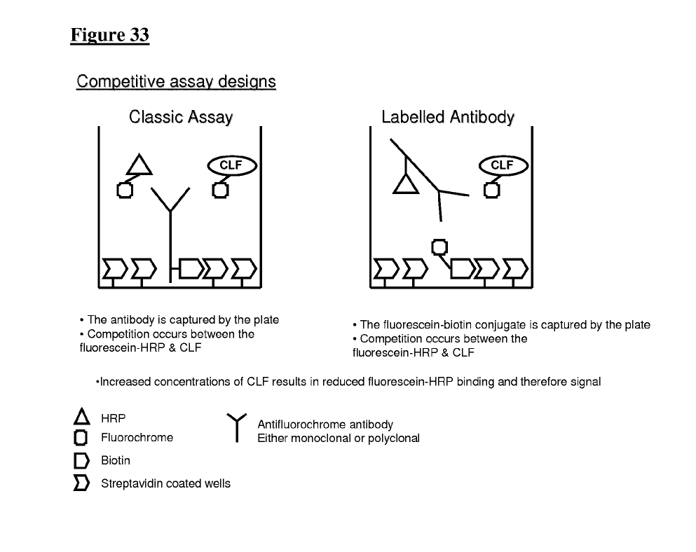

Two independent competitive immunoassay formats were developed and experimentally tested. The first (Assay format 1) comprises a biotin conjugated anti-FITC (fluorescein isothiocyanate) antibody (either monoclonal or polyclonal), a FITC-Horse Radish Peroxidase (HRP) conjugate, CLF (fluorescein (FITC)-conjugated bile salt) and a streptavidin coated ELISA microtitre plate. In this assay format the biotinalyated antibody is captured by the streptavidin coated ELISA plate. Competition for antibody binding occurs between fluorescein (FITC)-conjugated bile salt and the FITC-HRP conjugate. The amount of FITC-HRP can then be amplified and quantified directly through an enzymatic colorimetric reaction using tetramethylbenzidine (TMB) substrate. For the second assay (Assay format 2), the anti-FITC antibody was conjugated to HRP (anti-FITC-HRP) and the FITC conjugated to biotin (FITC-biotin). In this format the FITC-biotin conjugates are captured on the biotinylated ELISA plate (rather than the antibody in Assay format 1). The fluorescein (FITC)-conjugated bile salt and FITC-biotin conjugate compete for binding to the anti-FITC-HRP antibody. Unbound antibody is removed and the amount of remaining antibody quantified colorimetrically using tetramethylbenzidine (TMB) substrate. Standard curves can then be established with known concentrations of fluorescein (FITC)-conjugated bile salt which will compete for FITC-HRP or anti-FITC-HRP binding and allow the quantification of an unknown fluorescein (FITC)-conjugated bile salt in a biological fluid to be defined (see FIG. 33).

Preliminary experimental data quantifying CLF in PBS (phosphate buffered saline) showed that both assay formats (1 and 2) were functional, however testing in serum samples showed significantly lower recovery (<60% compared to PBS) which suggested significant protein binding and higher than acceptable inter-serum variation. A number of different agents were experimental tested to establish whether binding of the fluorescein-conjugated bile salt to serum proteins could be blocked. Agents tested included 8-anilino-1-naphthalene sulfonic acid (ANS, 0.2%), deoxycholic acid, cholic acid, low/high pH, phenol red and Orange G. ANS and deoxycholic acid showed the best blocking characteristics in Assay format 1, but the ANS blocking agent showed superior characteristics and was adopted. Both immunoassay formats were then compared using the ANS blocking substrate. Assay format 2 (HRP-anti-FITC antibody) showed superior recovery and accuracy and was therefore adopted and further optimised.

In parallel to developing the immunoassay, a second group has developed a quantitative HPLC method for CLF in plasma samples. Comparison of the two methods initially showed a 40% difference between the two methods, which was shown to originate from the method used to re-suspend CLF. CLF samples prepared using methanol (100%) showed a quantitative value approximately 40% higher than CLF prepared in aqueous ethanol (EtOH:H₂O:NaOH (50%) at a ratio of 500:1000:30) using both immunoassay and the HPLC methodologies. Determination of CLF in plasma samples using a standard curve prepared with known concentrations of CLF dissolved in 100% methanol is accurate in terms of expected and determined values. This is a novel and unexpected observation and shows that the correct preparation of CLF is absolutely required to obtain diagnostically relevant data in either animal or human studies.

### DEFINITIONS

The term “fluorescent conjugated bile salt” or “fluorescent conjugated bile salt derivatives” can mean a bile salt covalently bound to a fluorescent composition.

The term “detecting” is used in the broadest sense to include both qualitative and quantitative measurements of a target molecule.

The term “biological fluid” refers to a body sample from any animal, but preferably is from a mammal, more preferably from a human. In certain embodiments, such a biological sample is from a patient, prior to or following a given drug treatment. Such samples include biological fluids, such as serum, plasma, vitreous fluid, lymph fluid, whole blood, urine, bile and tissue culture medium, as well as tissue extracts, such as homogenized tissue and cellular extracts. In certain embodiments, the sample is a body sample from any animal. In one embodiment, it is from a mammal. In one embodiment, it is from a human subject. In one embodiment, such a biological sample is from clinical patients.

A “subject” may be a vertebrate, mammal, or domestic animal. The subject may be an experimental animal, such as a mouse or rat. Preferably, the subject is any mammal, for example livestock (e.g. a horse, a dog), pets, or may be used in other veterinary applications. Most preferably, however, the subject is a human being.

The term “detectable antibody” refers to an antibody that is capable of being detected either directly through a label amplified by a detection means, or indirectly through, e.g., another antibody that is labelled. For direct labelling, the antibody is typically conjugated to a moiety that is detectable by some means. In one embodiment, the detectable antibody is biotinylated antibody.

The term “detection means” refers to a moiety or technique used to detect the presence of the detectable antibody in the ELISA herein and includes detection agents that amplify the immobilized label, such as label captured onto a microtiter plate. In one embodiment, the detection means is a colorimetric detection agent such as avidin or streptavidin-HRP.

The term “capture reagent” refers to a reagent capable of binding and capturing a target molecule in a sample such that under suitable conditions, the capture reagent-target molecule complex can be separated from the rest of the sample. Typically, the capture reagent is immobilized or immobilizable. In a sandwich immunoassay, the capture reagent is preferably an antibody or a mixture of different antibodies against a target antigen.

The term “antibody” herein is used in the broadest sense and specifically covers intact monoclonal antibodies, polyclonal antibodies, multispecific antibodies (e.g. bispecific antibodies) formed from at least two intact antibodies, and antibody fragments so long as they exhibit the desired biological activity. Examples of antibody fragments include Fab, Fab′, F(ab′)2, and Fv fragments; diabodies; linear antibodies; single-chain antibody molecules; and multispecific antibodies formed from antibody fragments.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which:—

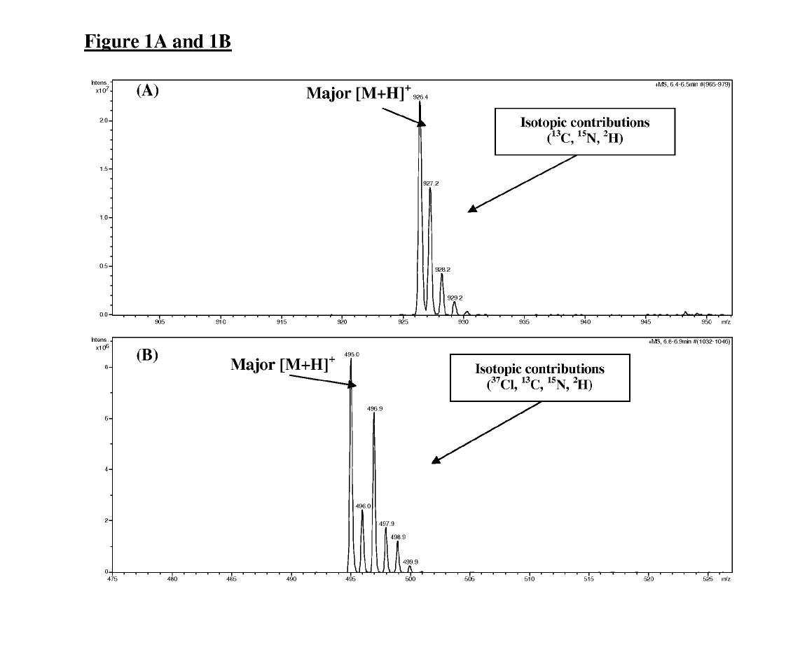

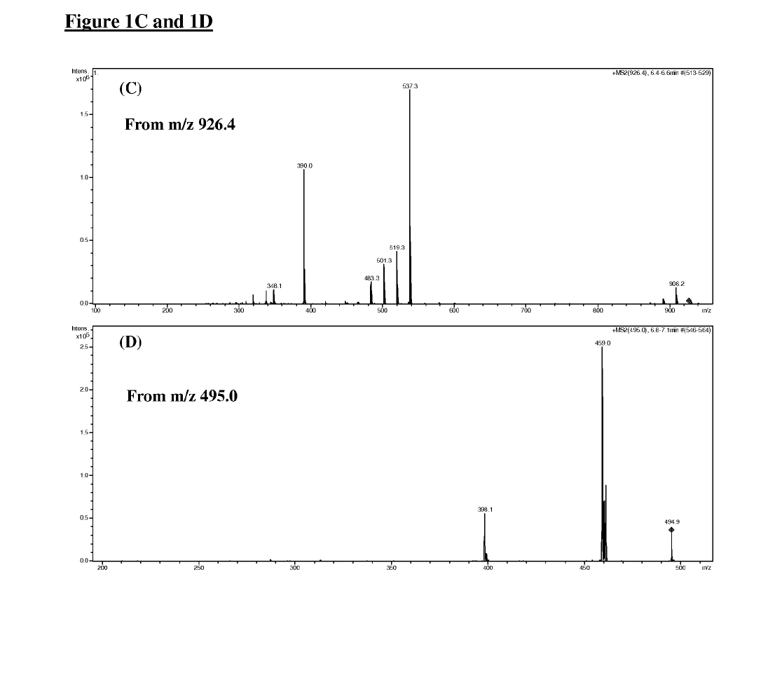

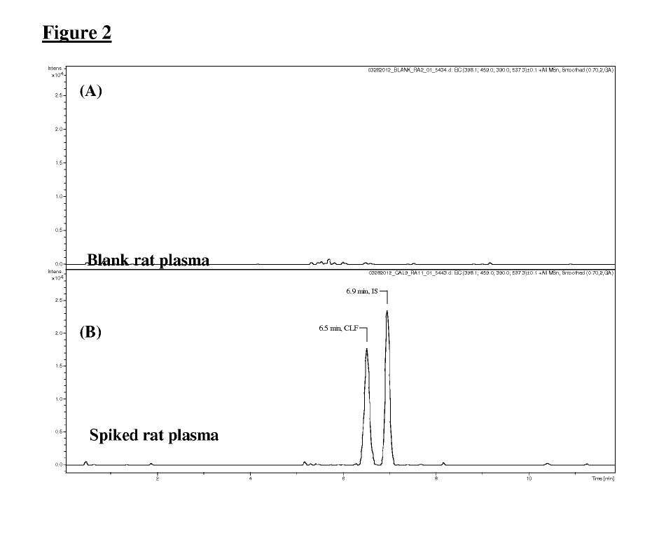

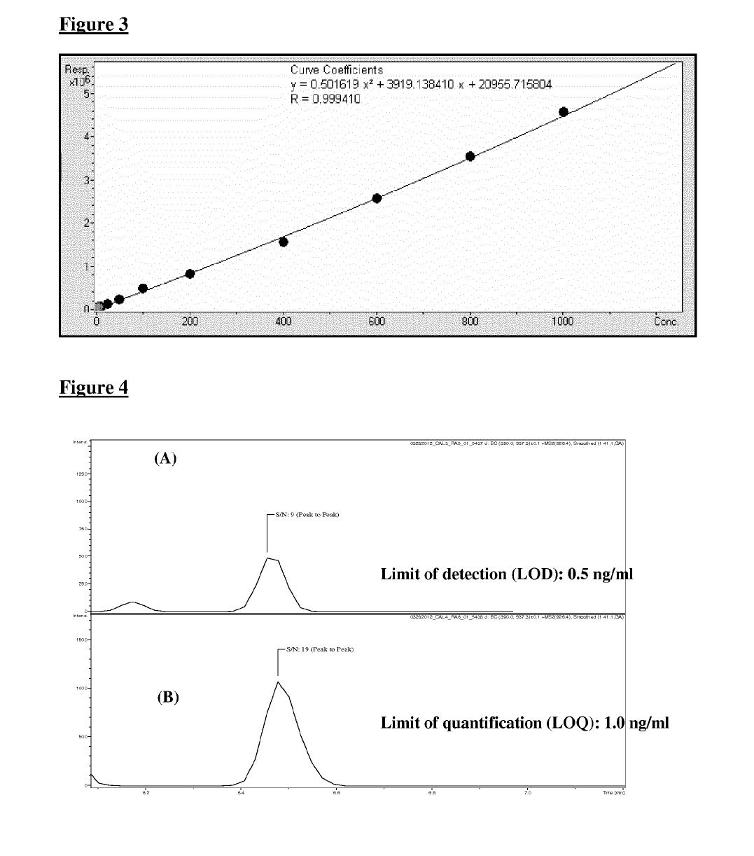

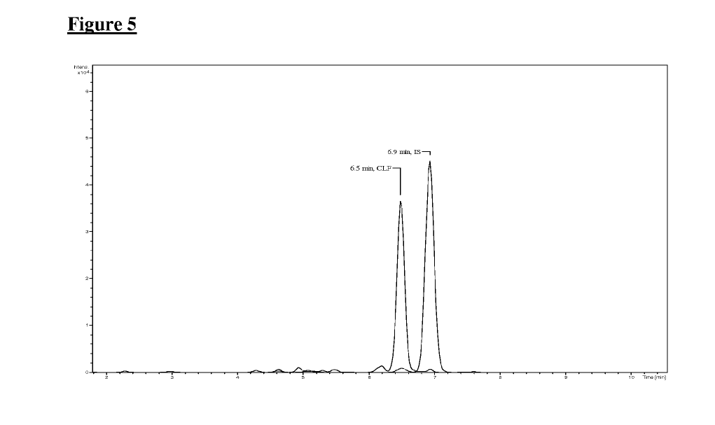

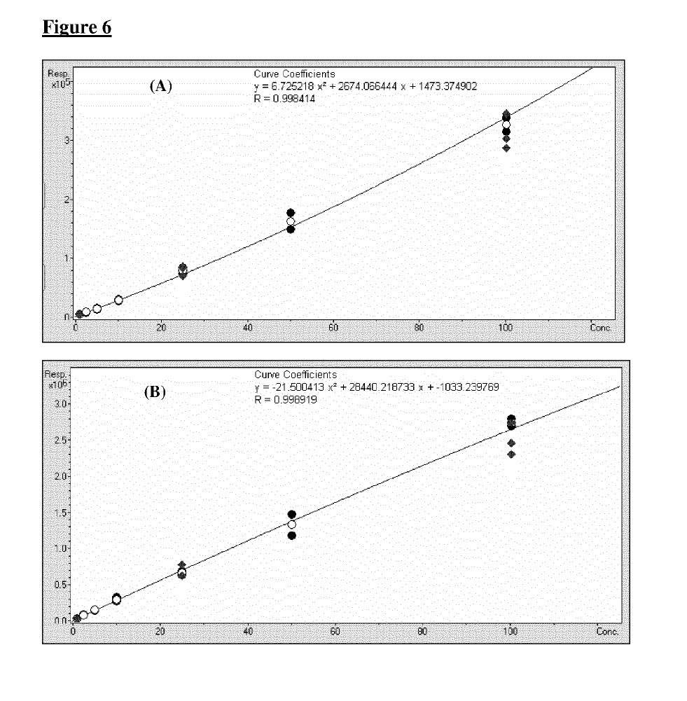

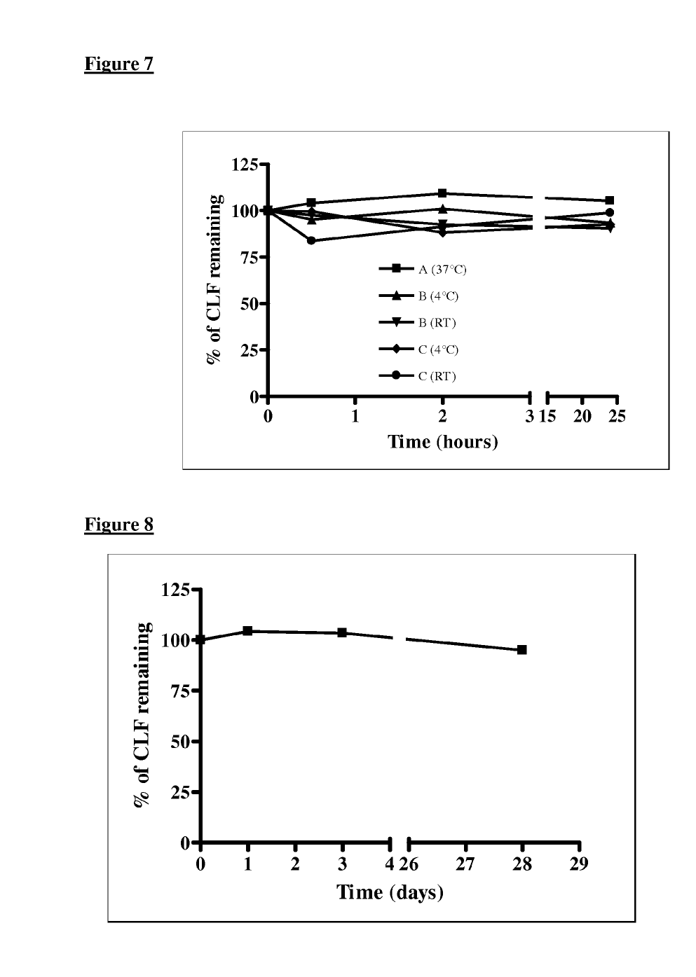

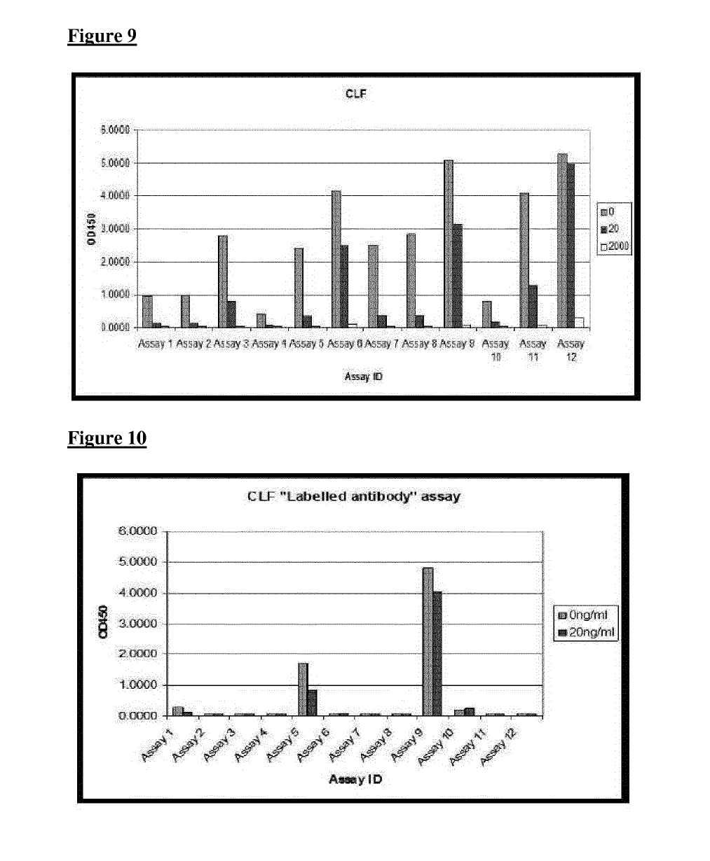

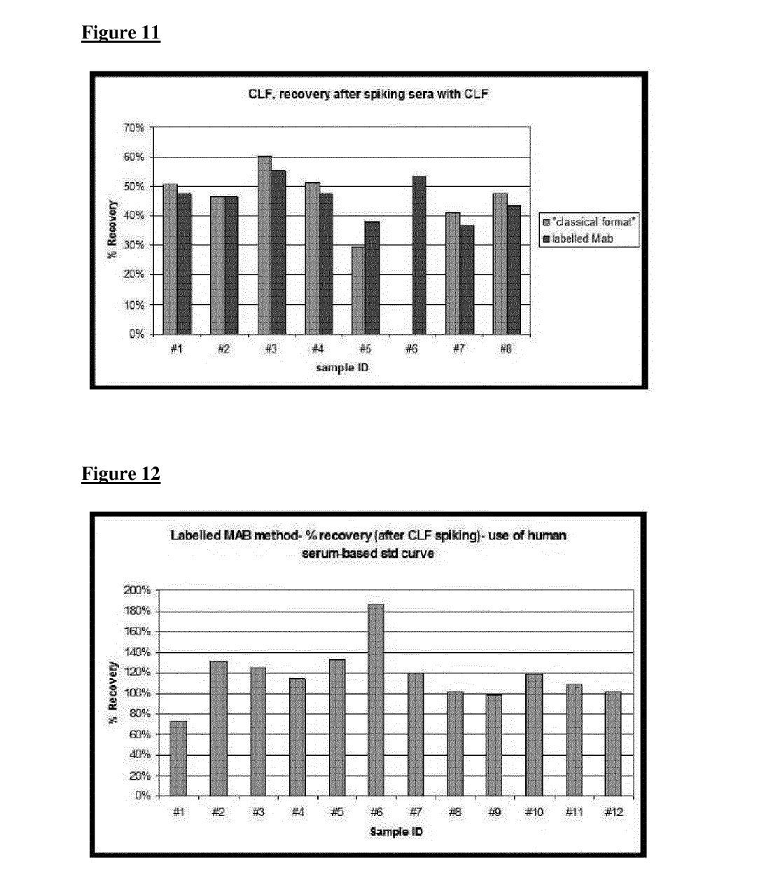

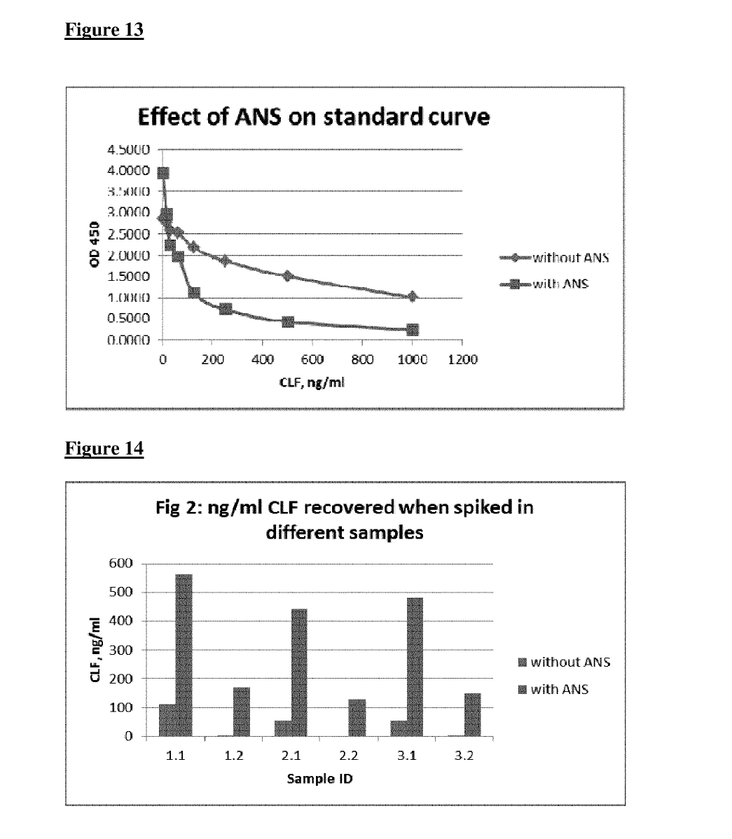

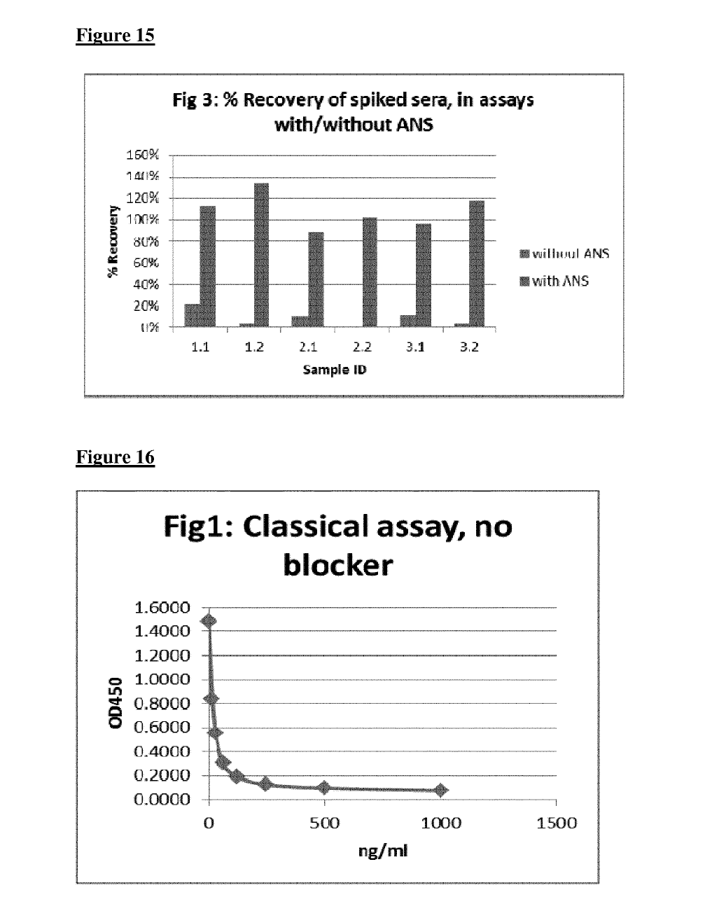

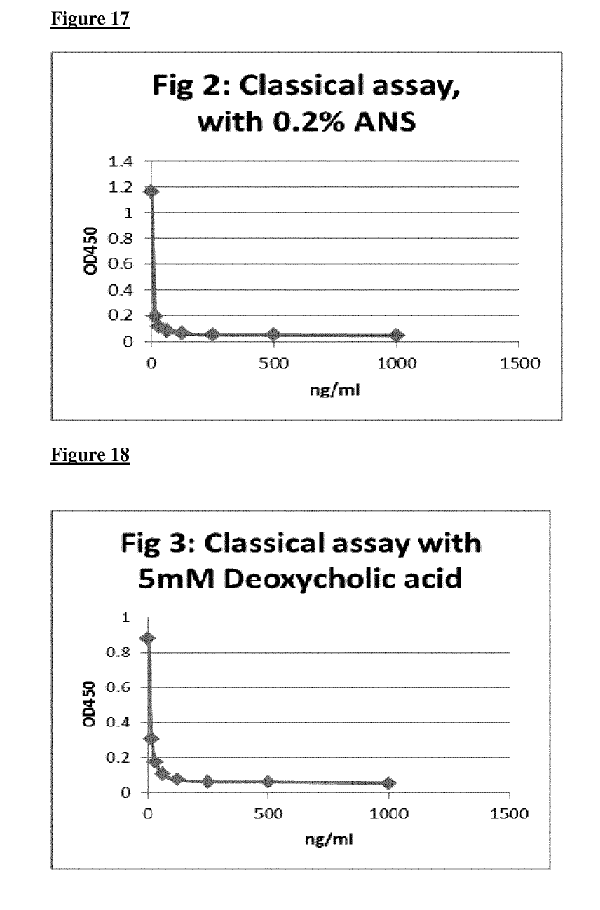

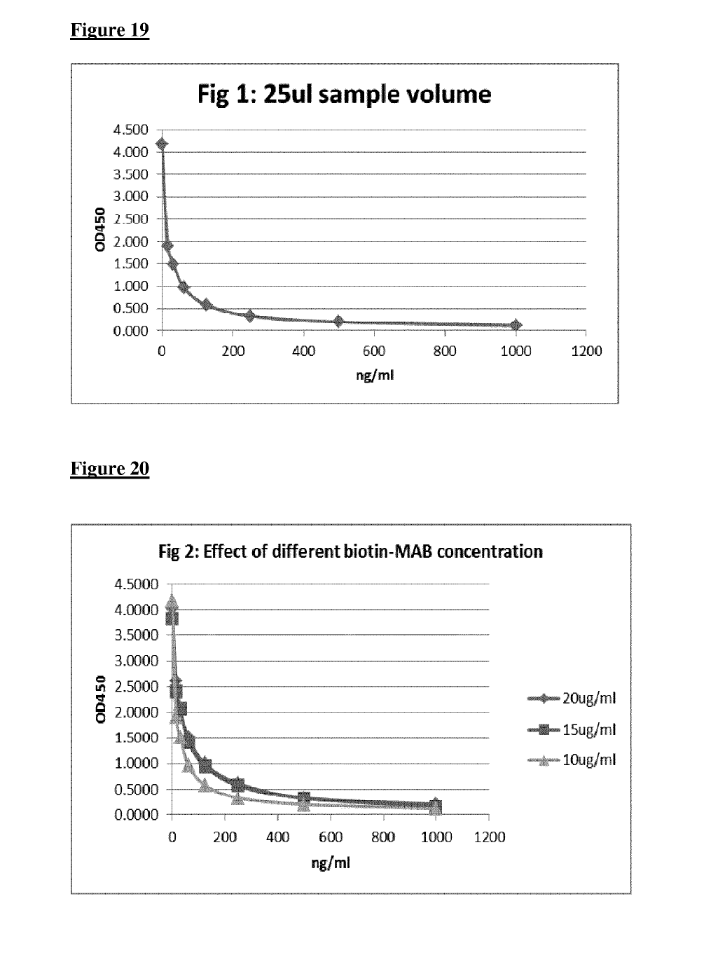

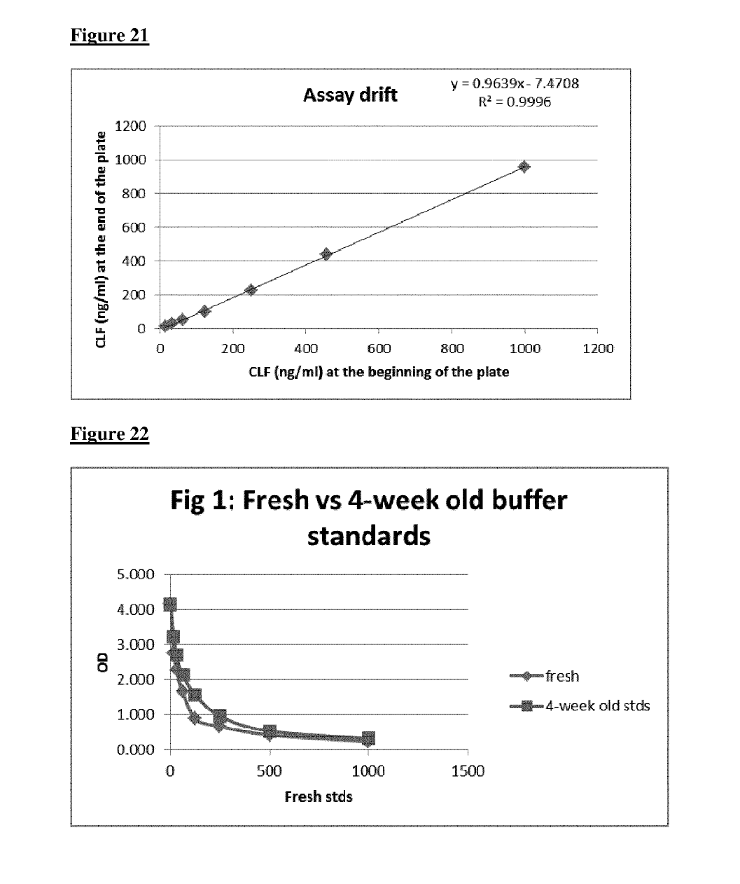

FIG. 1 (A) is a mass spectrum of CLF (m/z 926.4, [M+H]⁺), (B) is a mass spectrum of IS (m/z 495.0, [M+H]⁺), (C) is daughter ion spectra of CLF from m/z 926.4 and (D) is daughter ion spectra of IS from m/z 495.0;

FIG. 2 (A) is an EIC of rat blank plasma sample and (B) EIC of rat plasma sample spiked with CLF (too ng/ml) and IS (500 ng/ml). Transitions used for EIC of CLF: m/z 926.4>537.3+390.0. Transitions used for EIC of IS: m/z 495.0>459.0+398.1;

FIG. 3 is a calibration curve of CLF in rat plasma;

FIG. 4 is (A) an EIC of rat plasma sample spiked with 0.5 ng/ml of CLF and (B) EIC of rat plasma sample spiked with 1 ng/ml of CLF. Transitions used for EIC of CLF: m/z 926.4>537.3+390.0;

FIG. 5 is an EIC of a human plasma sample spiked with CLF (too ng/ml) and IS (2 μg/ml);

FIG. 6 is mean calibration curves and QCs of CLF obtained in human plasma during (A) the first and (B) the second experiment;

FIG. 7 shows the stability of CLF at 100 ng/ml in human blood. (A) Incubation of CLF in whole blood at 37° C., (B) Pre-incubation of CLF in whole blood at 37° C. during 30 minutes before preparation and incubation of plasma at room temperature and 4° C. and (C) incubation of CLF in plasma at room temperature and 4° C.

FIG. 8 shows the stability of CLF at 100 ng/ml in human plasma at −70° C. over 4 weeks;

FIG. 9 shows the results of a classical competitive ELISA performed under various conditions described within table 12;

FIG. 10 shows the results of a labelled-antibody competitive ELISA performed under various conditions described within table 13;

FIG. 11 shows the % recovery of CLF from sera samples spiked with CLF and detected using a classical competitive ELISA and a labelled-antibody competitive ELISA;

FIG. 12 shows the % recovery of CLF from sera samples spiked with CLF and detected using a labelled-antibody competitive ELISA with human sera present in the standard curve samples;

FIG. 13 shows the effect of ANS on the standard curve of a labelled-antibody competitive immunoassay detecting CLF;

FIG. 14 shows the recovery of CLF within human plasma samples spiked with CLF (moo ng/ml) in the presence and absence of ANS. CLF was detected using a labelled-antibody competitive immunoassay;

FIG. 15 is the percentage recovery of data displayed in FIG. 14. CLF was detected using a labelled-antibody competitive immunoassay;

FIG. 16 shows standard curve a of classical competitive immunoassays for CLF performed in the absence of a blocker;

FIG. 17 shows standard curve of a classical competitive immunoassays for CLF performed in the presence of 0.2% ANS;

FIG. 18 shows standard curve of a classical competitive immunoassays for CLF performed in the presence of 5 mM Deoxycholic acid;

FIG. 19 is a standard curve of a labelled-antibody competitive immunoassay for CLF performed with 25 μl samples;

FIG. 20 shows the standard curves of labelled-antibody competitive immunoassay for CLF performed increasing concentrations of biotin-MAB;

FIG. 21 is a line graph showing the extent of assay drift at both ends of a microtitre plate on which CLF was detected using a labelled-antibody competitive assay;

FIG. 22 shows the standard curve of a labelled-antibody immunoassay performed with fresh and 4-week old standard samples, which had been stored at 2-8° C.;

FIG. 23 is a linear regression between the concentration of CLF in fresh and 4-week old standard samples, which had been stored at 2-8° C.;

FIG. 24 is a line graph showing the observed vs expected concentrations of CLF detected when using a labelled-antibody competitive immunoassay. CLF standard stock samples were either diluted in methanol or aqueous ethanol;

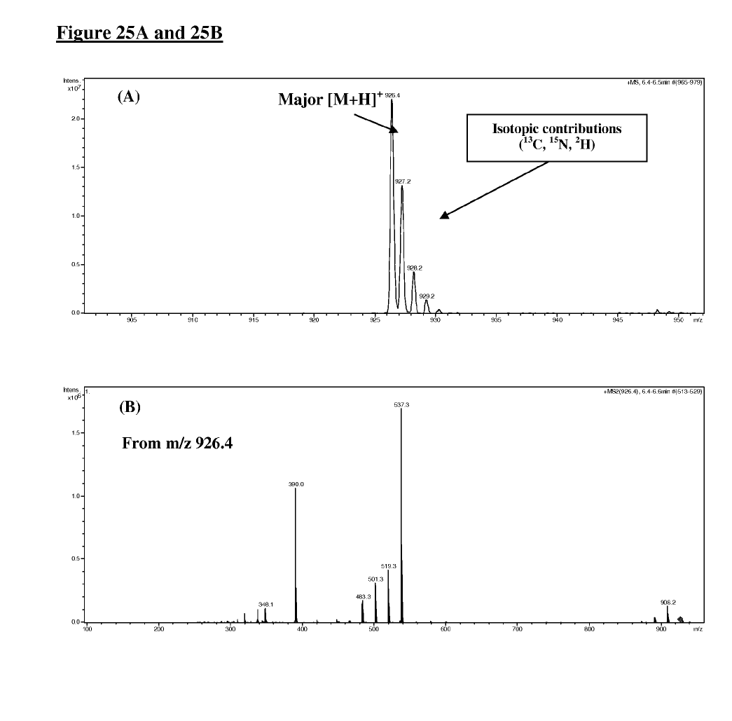

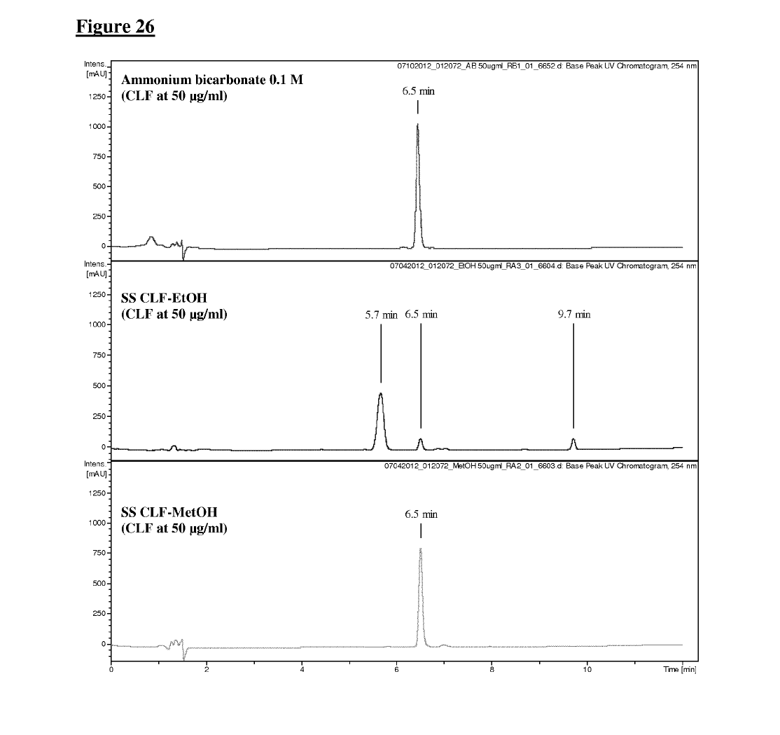

FIG. 25 is (A) mass spectrum of CLF (m/z 926.4, [M+H]⁺) and (B) Daughter ion spectra of CLF from m/z 926.4;

FIG. 26 is a HPLC-UV analysis of working and ammonium bicarbonate solutions (254 nm);

FIG. 27 is a mass spectrum obtained at 5.7 min (A) and 6.5 min (B);

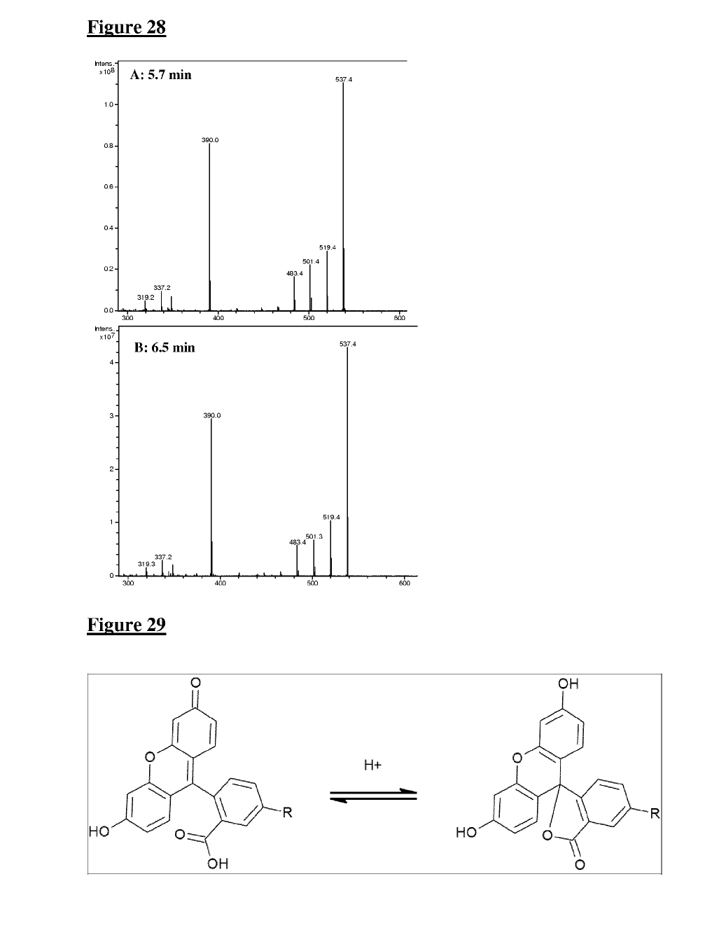

FIG. 28 is a MS/MS spectrum obtained at 5.7 min (A) and 6.5 min (B) from m/z 926.5 as parent ion;

FIG. 29 shows the tautomerization of the fluorescein moiety of CLF;

FIG. 30 is a representative chromatogram of CLF (1 μg/ml) in standard samples. Human plasma was spiked with concentrated solutions of CLF obtained from working solutions named SS CLF-EtOH (A) and SS CLF-MetOH (B);

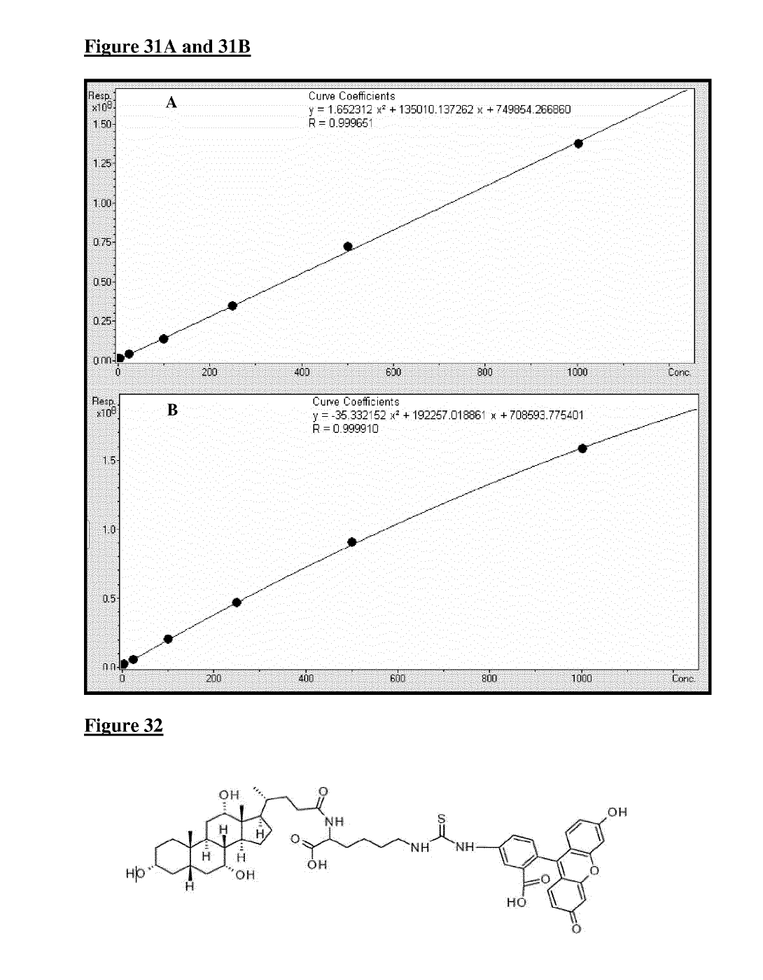

FIG. 31 are HPLC calibration curves of CLF in human plasma obtained from working solutions named SS CLF-MetOH (A) and SS CLF-EtOH (B);

FIG. 32 is the chemical structure of Cholyl-L-Lysyl-Fluorescein (CLF); and

FIG. 33 is a schematic diagram of a classical competitive and a competitive labelled-antibody immunoassay for CLF.

### EXAMPLES

### Example 1

### Development of a Sensitive Method for the Quantification of Cholyl-Lysyl-L-Fluorescein (CLF) in plasma by liquid chromatography tandem mass spectrometry [Formerly Appendix A]

### Summary

A high performance liquid chromatography tandem mass spectrometry assay was developed for the analysis of Cholyl-Lysyl-L-Fluorescein (CLF) and an internal standard (IS), 5-([4,6-Dichlorotriazin-2-yl]amino)fluorescein, from rat plasma samples. The method involves precipitation of proteins with acetonitrile and subsequent analysis by reversed phase liquid chromatography with mass spectrometry detection and positive electrospray ionization. The elution of compounds was achieved within 12 min on an Acclaim 120 C₁₈ column by elution with a linear gradient of acetonitrile/water/formic acid (0.1%). For a plasmatic concentration of CLF at 500 ng/ml, the extraction method allowed to obtain 111.3±9.7% of recovery (mean±SEM, n=3) and to minimize matrix effect to 116.8%. In our conditions of use, recovery and matrix effect obtained for IS (Internal Standard) were of 46.3±7.5% (mean±SEM, n=3) and 111.9%, respectively. The assay was found linear in the concentration range from 1 to 1000 ng/ml. The accuracy values, expressed by the bias between theoretical and back-calculated concentrations, were into the range −8.2-15.3% over the tested concentration levels, within our acceptance criteria. Limit of detection and limit of quantification were evaluated at 0.5 and 1.0 ng/ml, respectively. Thus, the developed method provides the rapid, easy, sensitive and selective requirement to quantify CLF in rat plasma or plasma of any other mammalian origin. This method could also be upgraded to quantify CLF in other biological matrices.

### Introduction

Cholyl-Lysyl-L-Fluorescein is a fluorescent bile salt derivative that is being developed as an agent in liver research (Mills et al., 1997). Cholyl-Lysyl-L-Fluorescein is classically quantified in biological matrices by measuring the fluorescence (Mills et al., 1997) with some limitations as pH dependence of the fluorescence quantum yield (Mills et al., 1997). Thus, the aim of this study was to develop a sensitive method for the quantification of Cholyl-Lysyl-L-Fluorescein in plasma by high performance liquid chromatography tandem mass spectrometry.

Prior to this study, the poor degree of sensitivity using established methods had not been fully appreciated in the art, and this study now demonstrated unexpectedly superior results with regards to assay sensitivity and accuracy.

### Materials and Methods

Reagents CLF and its IS, 5-([4,6-Dichlorotriazin-2-yl]amino)fluorescein hydrochloride, were purchased from Becton-Dickinson (France) and Sigma Aldrich (France), respectively (see Table 1). Water and acetonitrile (LC-MS grade) were obtained from Carlo Erba (France). Formic acid, methanol (100%), isopropanol (100%) and perchloric acid (70%) were purchased from Sigma Aldrich (France). Free-drug rat plasma was prepared from 8 adult male rats Sprague-Dawley obtained from Elevage Janvier (France).

TABLE 1
Structure of test compounds

| | | Monoisotopic |
|---|---|---|
| Compound | Molecular formula | mass (g/mol) |
| CLF | 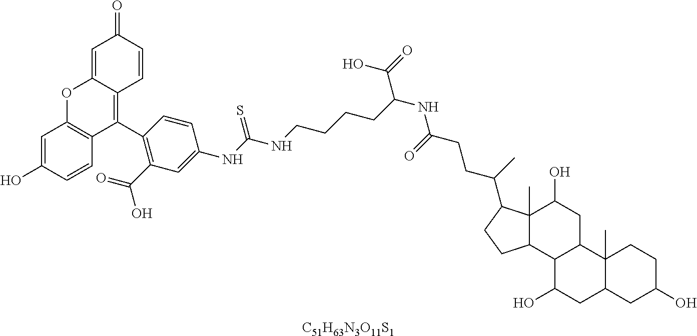 [SMILES: CC(CCC(=O)NC(CCCCNC(=S)Nc1ccc(-c2c3ccc(=O)cc-3oc3cc(O)ccc23)c(C(=O)O)c1)C(=O)O)C1CCC2C3C(O)CC4CC(O)CCC4(C)C3CC(O)C12C ; "C51H63N3O11S1"] | 925.42 |
| Internal standard (IS) | 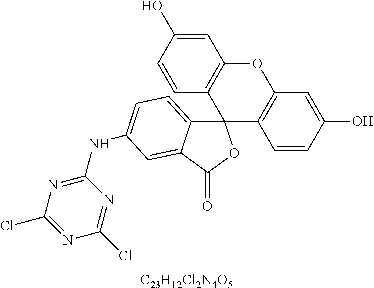 [SMILES: "C23H12Cl2N4O5" ; O=C1OC2(c3ccc(O)cc3Oc3cc(O)ccc32)c2ccc(Nc3nc(Cl)nc(Cl)n3)cc21] | 494.02 |

### Materials

Analyses were performed on a HPLC-MS/MS system consisting of an AmaZon SL ion trap mass spectrometer (Bruker) equipped with an ESI interface and an Ultimate 3000 device as HPLC system (Dionex) equipped with a thermostated autosampler and a thermostated column compartment. Data were acquired and processed using Hystar and its extension QuantAnalysis (version 3.2, Bruker), respectively.

### Chromatographic and Mass Spectrometric Conditions

HPLC separation was operated with a mixture of water and acetonitrile containing 0.1% of formic acid and the reversed phase mode was selected. The injected volume of sample was 20 μl and the “full loop” mode was applied. The temperature of the analytical column was kept at 30° C.

CLF and its IS were separated on an Acclaim 120 C₁₈ column (5 μm, 2.1×100 mm, Dionex) by elution with a linear gradient of acetonitrile in water as described in Table 2.

An ion trap mass spectrometer equipped with an ESI interface was operated in the positive ionization mode. For each compound, the most abundant ion formed in MS was selected for MS/MS fragmentations. In order to increase the sensitivity and the specificity of the method, the MRM mode was selected. The most specific daughter ions formed in MS/MS were used for quantification.

The conditions for the HPLC-MS/MS are presented in Table 2.

TABLE 2
Summary of HPLC-MS/MS conditions

| | |
|---|---|
| Column | Acclaim 120 C₁₈ column (5 μm, 2.1 × 100 mm, |
| | Dionex) |
| Mobile phase A | 0.1% formic acid in 5/95 (acetonitrile/water; |
| | v/v) |
| Mobile phase B | 0.1% formic acid in acetonitrile |
| Linear gradient | From 20 to 100% of mobile phase B in |
| | 8 minutes |
| | Then, B = 100% during 2 min |
| | (8-10 minutes) |
| | Then, from 100 to 20% of mobile phase B in |
| | 2 minutes |
| | Total run time: 12 min |
| Flow rate | 200 μl/min |
| Injection volume | 20 μl in full loop mode |
| Column temperature | 30° C. |
| Polarity | Positive ESI |
| Capillary voltage | 4.5 kV |
| Dry gas flow | 8 l/min |
| Dry temperature | 200° C. |
| Nebulizer pressure | 15 psi |
| Desolvatation gas | Dinitrogen N₂ |
| CID gas | Helium |
| MS/MS transitions | CLF: m/z 926.4 > 537.3 and 926.4 > 390.0 |
| | IS: m/z 495.0 > 459.0 and 495.0 > 398.1 |

### Recovery and Matrix Effect Evaluation

Recovery and matrix effect were evaluated in plasma at one concentration level (500 ng/ml) in three replicates. Four solvents were assayed to precipitate the plasmatic proteins: acetonitrile, methanol, isopropanol and perchloric acid 7%.

Three standard samples were prepared at 500 ng/ml by adding 10 μl of a 10-fold concentrated solution of CLF in methanol (5 μg/ml) to 90 μl of free-drug rat plasma. These samples were extracted by adding 100 μl of solvent containing 1 μg/ml of IS. Then, samples were vortex-mixed and centrifuged at 20,000 g for 5 min (4° C.). The supernatant (150 μl) was transferred in 1.2 ml HPLC glass vials and sealed.

One recovery sample was prepared by adding 10 μl of methanol to 90 μl of free-drug rat plasma. This sample was extracted by adding 100 μl of solvent without IS. Then, samples were vortex-mixed and centrifuged at 20,000 g for 5 min (4° C.). After centrifugation, 127.5 μl of supernatant were spiked with 7.5 μl of the 10-fold concentrated solution of CLF in methanol and 15 μl of an IS solution at 5 μg/ml in solvent. The final quantities of CLF and IS were theoretically close to those of extracted standard samples. Sample was transferred in 1.2 ml HPLC glass vials, sealed, placed into the refrigerated autosampler and 20 μl were injected to the HPLC-MS/MS system.

One control sample was prepared by adding 10 μl of a 10-fold concentrated solution of CLF in methanol to 90 μl of water and then 100 μl of solvent containing 1 μg/ml of IS were added. The final quantities of CLF and IS were theoretically close to those of extracted standard samples. Sample was transferred in 1.2 ml HPLC glass vials, sealed, placed into the refrigerated autosampler and 20 μl were injected to the HPLC-MS/MS system.

Recovery was calculated as follows:

$$\frac{( Peak area of compound in standard sample ) \times 100}{( Peak area of compound in recovery sample )}$$

Matrix effect was calculated as follows:

$$\frac{( Peak area of compound in recovery sample ) \times 100}{( Peak area of compound in control sample )}$$

### Calibration Curve Preparation

A standard stock solution of CLF at 100 μg/ml was prepared in methanol as described above. Then, this stock solution was serially diluted in methanol in order to obtain fourteen 10-fold concentrated solutions of CLF (from 1 to 10000 ng/ml).

The calibration curve consisted of fourteen single points and was generated by adding 10 of 10-fold required concentrated solutions of CLF to 90 μl of free-drug rat plasma. The final concentrations of CLF in plasma were 1000, 800, 600, 400, 200, 100, 50, 25, 10, 5, 1, 0.5, 0.25 and 0.1 ng/ml. A blank sample was prepared by adding 10 μl of water to 90 μl of free-drug rat plasma.

A solution of IS at 1 μg/ml in acetonitrile was prepared. Then, 100 μl of this solution was added to 100 μl of standard samples in order to precipitate the proteins. The blank sample was extracted by 100 μl of acetonitrile without IS. Then, Samples were vortex-mixed and centrifuged at 20,000 g for 5 min (4° C.). The supernatant (150 μl) was transferred in 1.2 ml HPLC glass vials and sealed. Samples were placed into the refrigerated autosampler and 20 μl were injected to the HPLC-MS/MS system.

### Method Development

The method was developed by carrying out a test of linearity, an evaluation of LOQ and LOD, and a comparison of recovery and matrix effect obtained with classical protein precipitation methods using four distinct solvents.

### Results

### Chromatographic and Mass Spectrometric Conditions

The selected mass spectrometric conditions led to the production of specific daughters ions from a specific [M+H]⁺ parent ion for both compounds, as shown in FIG. 1. The parent ions formed were detected at m/z 926.4 and m/z 495.0 for CLF and its IS, respectively. Two major daughter ions were detected at m/z 537.3 and m/z 390.0 for CLF (from m/z 926.4) and 2 major daughter ions were detected at m/z 459.0 and m/z 398.1 for IS (from m/z 495.0). The hypothetic chemical structures of CLF specific daughter ions formed are available in Table 3.

TABLE 3
CLF daughter ions formed in mass spectrometer (from m/z 926.4) and used for detection and quantification.

| [M + H]⁺ | Hypothetic chemical structure (no | Theoretical |
|---|---|---|
| (m/z) | charged form) | m/z of [M + H]⁺ |
| 537.3 | 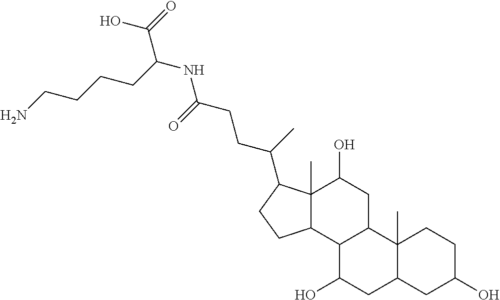 [SMILES: CC(CCC(=O)NC(CCCCN)C(=O)O)C1CCC2C3C(O)CC4CC(O)CCC4(C)C3CC(O)C12C] | 537.39 |
| 390.0 | 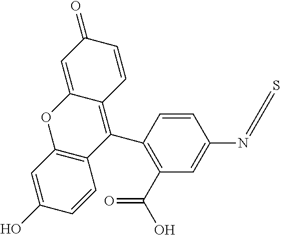 [SMILES: O=C(O)c1cc(N=C=S)ccc1-c1c2ccc(=O)cc-2oc2cc(O)ccc12] | 390.04 |

The selected HPLC conditions allowed a clear-cut separation of the CLF and its IS, as shown by their retention times (see FIG. 2). Retention times were of 6.5 and 6.9 min for CLF and its IS, respectively. Chromatograms obtained with rat plasma samples showed the absence of interfering peaks using the selected MRM mode (see FIG. 2).

### Recovery and Matrix Effect Evaluation

The four solvents were compared with each other in terms of recovery and matrix effect. A matrix effect can be observed as ion suppression, in cases where values were lower than 100%, and as ion enhancement, in cases where values were found higher than 100%. Recovery reflects the ability of the extraction method to extract compounds from their matrix.

Results obtained with the four solvents tested are summarized in Table 4.

TABLE 4
Comparison between 4 protein precipitation methods

| | | Recovery (%) | Matrix effect |
|---|---|---|---|
| Compounds | Solvent | Mean ± SEM, n = 3 | (%) |
| CLF | Acetonitrile | 111.3 ± 9.7 | 116.8 |
| | Methanol | 87.6 ± 5.1 | 36.7 |
| | Isopropanol | 110.5 ± 9.4 | 71.3 |
| | Perchloric acid 7% | 1.1 ± 0.1 | 96.2 |
| IS | Acetonitrile | 46.3 ± 7.5 | 111.9 |
| | Methanol | 37.8 ± 6.3 | 0.2 |
| | Isopropanol | 43.1 ± 3.0 | 45.6 |
| | Perchloric acid 7% | 9.8 ± 0.6 | 130.9 |

Thus, acetonitrile was selected as the extraction solvent. Indeed, it offered the most satisfactory results in terms of recovery and matrix effect for both CLF and its IS.

### Linearity of the Method

Chromatographic peak analysis was performed by QuantAnalysis software. Area under chromatographic peaks was calculated for CLF and its IS. The peak area ratio CLF/IS was calculated and used as signal response. The quadratic regression model (1/x² weighting; origin excluded) was used for quantification. Calibration levels at 0.1, 0.25 and 0.5 ng/ml were exclude because they were below the limit of quantification (S/N ratio lower than 10). Calibration level at 5 ng/ml was excluded for the evaluation of linearity because it was out of specification (bias >20%).

Linearity of the method was demonstrated by the r² value and the accuracy of each calibrator was reported with the bias between theoretical and back-calculated concentration at each level (see FIG. 3 and Table 5).

TABLE 5
Linearity of the method

| Theoretical | | Calculated | |
|---|---|---|---|
| concentration | | concentration | Accuracy |
| (ng/ml) | n | (ng/ml) | (%) |
| 1.00 | 1 | 1.01 | 0.7 |
| 5.00* | 1* | 8.02* | 60.4* |
| 10.00 | 1 | 9.18 | −8.2 |
| 25.00 | 1 | 24.90 | −0.4 |
| 50.00 | 1 | 50.40 | 0.8 |
| 100.00 | 1 | 115.31 | 15.3 |
| 200.00 | 1 | 194.91 | −2.5 |
| 400.00 | 1 | 370.66 | −7.3 |
| 600.00 | 1 | 596.17 | −0.6 |
| 800.00 | 1 | 805.19 | 0.6 |
| 1000.00 | 1 | 1016.31 | 1.6 |

*Exclude for the evaluation of linearity

Accuracies were into the range −8.2-15.3%, and the r² value was of 0.9998. Thus, the linearity of the method was successfully tested from 1 to 1000 ng/ml using 10 levels of concentration.

### Sensitivity of the Method

The sensitivity of the method was defined by the evaluation of the LOD and the LOQ obtained in rat plasma samples. The LOD and the LOQ were of 0.5 and 1 ng/ml and the signal to noise ratios obtained were 9 and 19 (Peak to Peak mode), respectively (see FIG. 4), thereby validating the LOD and the LOQ of the assay.

### Conclusions

The main drawbacks in the established art of CLF quantification in biological matrices by fluorescence are the pH dependence of the fluorescence quantum yield and the protein binding, such as binding of CLF to albumin (Mills et al., 1997). Indeed, the efficiency of fluorescence (that is, the quantum yield) is determined by the ratio between the number of emitted and absorbed photons by the molecule. This quantum yield may vary depending on the environment of fluorochromes (for example, concentration, polarity, pH) (Fayet M et al., 1971).

Thus, we have developed a new method for the quantification of CLF in rat plasma based on protein precipitation extraction followed by HPLC-MS/MS analysis and positive electrospray ionization. The developed method provides the rapid, easy, sensitive and selective requirement to quantify CLF in rat plasma or plasma of other origin. This method could also be upgraded to quantify CLF in other biological matrices.

### Example 2

### Stability of Cholyl-Lysyl-L-Fluorescein (CLF) in Human Blood by High Performance Liquid Chromatography Tandem Mass Spectrometry [Formerly Appendix B]

### Summary

A high performance liquid chromatography tandem mass spectrometry method was previously developed for the analysis of Cholyl-Lysyl-L-Fluorescein (CLF) and an internal standard (IS), 5-([4,6-Dichlorotriazin-2-yl]amino)fluorescein in human plasma (Report # CP-2012022). During the study described here, in vitro stability of CLF (at 100 ng/ml) was evaluated in human blood and plasma. CLF was found stable during 24 hours at 37° C. in whole human blood; Pretreatment of whole human blood with CLF at 37° C. followed by collection of plasma and incubation at room temperature and at 4° C. showed CLF to be stable, for up to 24 hours, under these conditions. CLF stability in human plasma was also evaluated and confirmed at −70° C. for at least 4 weeks. The relationship between concentrations used for spiked whole blood and CLF concentrations quantified in plasma (from 147.7 to 180.3 ng/ml) has suggested that CLF was mainly distributed in the plasmatic fraction following centrifugation of whole blood.

### Introduction

The aim of this study was to evaluate the stability of CLF in human blood at 37° C. for 30 min, 120 min and 24 h. Stability of CLF in human plasma was also studied at room temperature and 4° C. for 30 min, 120 min and 24 h. Stability of CLF in human plasma was also studied with pre-incubation of CLF in whole blood at 37° C. during 30 min before preparation and incubation of plasma. Finally, stability of CLF in our storage conditions (−70° C.) was evaluated over 4 weeks. Each sample was split in two equal fractions: one for ELISA analysis and one for HPLC-MS/MS analysis.

### Material and Methods

### Reagents

CLF and its internal standard (IS), 5-([4,6-Dichlorotriazin-2-yl]amino)fluorescein hydrochloride, were purchased from Becton-Dickinson (France) and Sigma Aldrich (France), respectively. Water and acetonitrile (both LC-MS grade) were obtained from Carlo Erba (France). Formic acid and methanol were purchased from Sigma Aldrich (France). Human EDTA blood was obtained from 3 individual consenting male donors by the “Etablissement Francais du Sang” (EFS, France). Human plasma was prepared from human whole blood by centrifugation of samples at 2000 g (4° C.) for 10 min.

### Materials

Incubation of samples was carried out using a water bath (PBX30, Jouan, France) at 37° C. and in a cold room at 4° C. Analyses were performed on a HPLC-MS/MS system consisting of an AmaZon SL ion trap mass spectrometer (Bruker) equipped with an ESI interface and an Ultimate 3000 device as HPLC system (Dionex) equipped with a thermostated autosampler and a thermostated column compartment. Data were acquired and processed using Hystar and its extension QuantAnalysis (version 3.2, Bruker), respectively.

### Preparation of Samples

### Test Samples

A working solution of CLF was prepared at 10 μg/ml by diluting the compound in methanol (100%).

### Test 1:

Fifty microliters (50 μl) of the working solution of CLF were added to 5 ml of human EDTA blood. The final concentration of CLF in blood was of 100 ng/ml. One aliquot of 1 ml of blood was taken (To) and the remaining solution was incubated at 37° C. in a water bath for 30 min, 120 min and 24 h. At the end of each incubation time, 1 ml of blood was taken and plasma was prepared by centrifugation of samples at 2000 g (4° C.) for 10 min. Two equal aliquots of 200 μl were prepared and stored at −70° C. until further analysis.

### Test 2:

Eighty microliters (80 μl) of the working solution of CLF were added to 8 ml of human EDTA blood. The final concentration of CLF in blood was of 100 ng/ml. Then, sample was incubated at 37° C. in water bath for 30 min and plasma was prepared as described previously. Plasma was then divided into two equal fractions of 2 ml each. One fraction was incubated at room temperature and the second one was incubated at 4° C. in a cold room. Two aliquots of 200 μl were taken from both fractions just before incubation and after 30 min, 120 min and 24 h of incubation. Samples were stored at −70° C. until further analysis.

Test 3: Forty microliters (40 μl) of the working solution of CLF were added to 4 ml of human plasma. The final concentration of CLF was of 100 ng/ml. Two aliquots of 200 μl of plasma spiked with CLF were taken (To) and the remaining solution was divided in 2 equal fractions. One fraction was incubated at room temperature and the second one was incubated at 4° C. Two aliquots of 200 μl were taken from both fractions after 30 min, 120 min and 24 h of incubation. Samples were stored at −70° C. until further analysis.

### Test 4:

At To and 4 weeks, 72 hours and 24 hours before To, 4 μl of the working solution of CLF were added to 400 μl of human plasma. The final concentration of CLF was of 100 ng/ml. Samples were divided in 2 equal fractions of 200 μl and stored at −70° C. until further analysis.

### Standard Samples

The HPLC-MS/MS analysis was conducted with two calibration curves, constituted with 7 single points (non zero), 9 quality controls (3 per level, 3 levels) and 2 zero points.

Standard solutions were prepared from a stock solution of CLF at 100 μg/ml prepared the day of the analysis. Each standard was prepared in drug-free human plasma from 10-fold concentrated solutions, prepared in methanol by serial dilution of the stock solution.

Three calibration curves were prepared by adding 20 μl of the concentrated standard solution (or 20 μl of methanol for zero samples) to 180 μl of free-drug human plasma. The final concentrations of calibrators were of 100, 50, 25, 10, 5, 2.5 and 1 ng/ml. The first calibration curve was injected at the beginning of analytical run, the second one at the end and the third one was stored for further analysis.

In the same way, 3 levels of quality controls (QC) were prepared in methanol in order to evaluate the precision and the accuracy of the analytical run. One, at the high level of the calibration curve (HQC, 100 ng/ml), one in the middle range (MQC, 25 ng/ml) and one at the low level of the calibration curve (LQC, 1 ng/ml). Each QC was prepared in 3 replicates. Three control samples were prepared at 100, 25 and 1 ng/ml by replacing free-drug human plasma with water. These samples allowed us to estimate the efficiency of the extraction method.

Recovery was calculated as follows:

$$\frac{( Peak area of compound in QC sample ) \times 100}{( Peak area of compound in control sample )}$$

### Extraction of Samples

An internal standard (IS), 5-([4,6-Dichlorotriazin-2-yl]amino)fluorescein hydrochloride, was added to each plasma sample (calibrators, QC, zero and test samples) in order to monitor the extraction. Thus, a stock solution of IS at 100 μg/ml in acetonitrile was 50-fold diluted in acetonitrile in order to obtain a final concentration of 2 μg/ml. Then, 200 μl of this solution were added to each plasma sample in order to precipitate the proteins. Samples were vortexed/mixed and centrifuged 5 min at 20,000 g (4° C.). After centrifugation, the clear supernatant (around 250 μl) was transferred into 1.2 ml HPLC glass vials and sealed. Samples were placed into the refrigerated autosampler and 20 μl were injected into the HPLC-MS/MS system. Test samples were injected twice.

### Chromatographic and Mass Spectrometric Conditions

HPLC separation was operated with a mixture of water and acetonitrile containing 0.1% of formic acid and the reversed phase mode was selected. The injected volume of sample was 20 μl and the “full loop” mode was applied. The temperature of the analytical column was kept at 30° C.

CLF and its IS were separated on an Acclaim 120 C₁₈ column (5 μm, 2.1×100 mm, Dionex) by elution with a linear gradient of acetonitrile in water as described in Table 6.

TABLE 6
Summary of HPLC-MS/MS conditions

| | |
|---|---|
| Column | Acclaim 120 C₁₈ column (5 μm, 2.1 × 100 mm, |
| | Dionex) |
| Mobile phase A | 0.1% formic acid in 5/95 (acetonitrile/water; |
| | v/v) |
| Mobile phase B | 0.1% formic acid in acetonitrile |
| Linear gradient | From 20 to 100% of mobile phase B in |
| | 8 minutes |
| | Then, B = 100% during 2 min (8-10 minutes) |
| | Then, from 100 to 20% of mobile phase B in |
| | 2 minutes |
| | Total run time: 12 min |
| Flow rate | 200 μl/min |
| Injection volume | 20 μl in full loop mode |
| Column temperature | 30° C. |
| Retention times | CLF: 6.5 min |
| | IS: 6.9 min |
| Polarity | Positive ESI |
| Capillary voltage | 4.5 kV |
| Dry gas flow | 8 l/min |
| Dry temperature | 200° C. |
| Nebulizer pressure | 15 psi |
| Desolvatation gas | Dinitrogen N₂ |
| CID gas | Helium |
| MS/MS transitions | CLF: m/z 926.4 > 537.3 and 926.4 > 390.0 |
| | IS: m/z 495.0 > 459.0 and 495.0 > 398.1 |

An ion trap mass spectrometer equipped with an ESI interface was operated in the positive ionization mode. For each compound, the most abundant ion formed in MS was selected for MS/MS fragmentations. In order to increase the sensitivity and the specificity of the method, the MRM mode was selected. The most specific daughter ions formed in MS/MS were used for quantification.

### Results

### Chromatographic and Mass Spectrometric Conditions

The selected mass spectrometric conditions led to the production of specific daughters ions from a specific [M+H]⁺ parent ion for both compounds, as shown in FIG. 1. The parent ions formed were detected at m/z 926.4 and m/z 495.0 for CLF and its IS, respectively. Two major daughter ions were detected at m/z 537.3 and m/z 390.0 for CLF (from m/z 926.4) and 2 major daughter ions were detected at m/z 459.0 and m/z 398.1 for IS (from m/z 495.0). The hypothetic chemical structures of CLF specific daughter ions formed are shown in Table 7.

TABLE 7
CLF parent and daughter ions formed in mass spectrometer and used for detection and quantification.

| | | Theoretical |
|---|---|---|
| [M + H]⁺ | Hypothetic chemical structure | m/z of |
| (m/z) | (no charged form) | [M + H]⁺ |
| 926.4 (parent ion) | 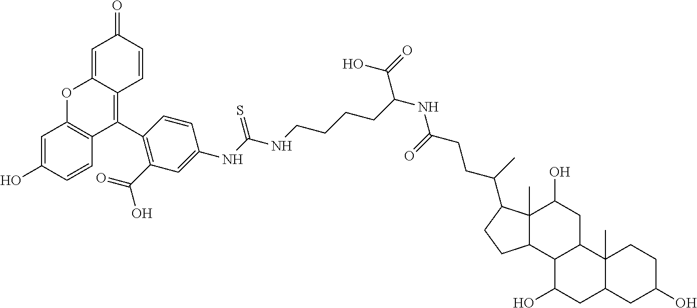 [SMILES: CC(CCC(=O)NC(CCCCNC(=S)Nc1ccc(-c2c3ccc(=O)cc-3oc3cc(O)ccc23)c(C(=O)O)c1)C(=O)O)C1CCC2C3C(O)CC4CC(O)CCC4(C)C3CC(O)C12C] | 926.43 |
| 537.3 (daughter ion) | 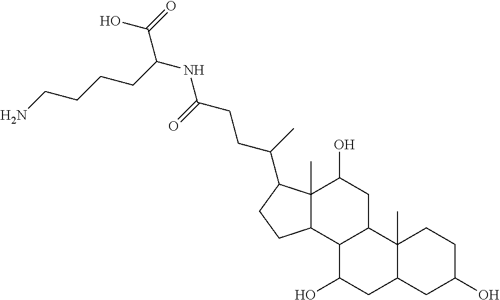 [SMILES: CC(CCC(=O)NC(CCCCN)C(=O)O)C1CCC2C3C(O)CC4CC(O)CCC4(C)C3CC(O)C12C] | 537.39 |
| 390.0 (daughter ion) | 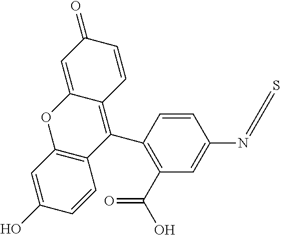 [SMILES: O=C(O)c1cc(N=C=S)ccc1-c1c2ccc(=O)cc-2oc2cc(O)ccc12] | 390.04 |

The selected HPLC-MS/MS conditions allowed a clear-cut for both separation and detection of the CLF and its IS, as shown by their retention times (see FIG. 5). Retention times were of 6.5 and 6.9 min for CLF and its IS, respectively.

### Evaluation and Validation of CLF Assay in Human Plasma

### Linearity of the Method

The mean back-calculated concentrations were processed with the 1/x² weighed quadratic regression model after analysis of four calibration curves consisting of seven concentration levels each obtained over two distinct experiments. The mean r² value of the mean curves obtained over the 2 experiments was calculated and was 0.9973±0.0007 (mean±SEM, n=2).

The linearity of the method was successfully tested from 1 to 100 ng/ml for CLF. At the limit of quantification (LOQ), the coefficient of variation (CVs) was 12.3%. For other concentration levels, the CVs obtained were lower than 12.4%. The mean absolute calculated concentrations (mean bias) did not deviate by more than 2.1% at each concentration level (Table 8 and FIG. 6).

### Precision and Accuracy of the Method

Intra- and inter-assay precisions and accuracy parameters were assessed using freshly prepared calibration curves and QC samples, obtained as described in Materials and Methods, and were calculated from two experiments of three replicates at three concentration levels (1, 25 and 100 ng/ml). At the LOQ, the intra- and inter-assay CVs and the accuracy (expressed with the mean bias) were 10.7%, 10.7% and 3.2%, respectively. For other concentration levels, the intra- and inter-assay CVs and the accuracy (expressed with the mean bias) did not exceed 11.6%, 13.1% and 6.8%, respectively (Table 9).

TABLE 8
Linearity of the method in human plasma over 2 experiments.

| Theoretical | | Mean calculated | | Mean |
|---|---|---|---|---|
| concentration | | concentration | CV | bias |
| (ng/ml) | n | (ng/ml) | (%) | (%) |
| 1.0 | 4 | 1.0 | 12.3 | 0.5 |
| 2.5 | 4 | 2.5 | 9.4 | −0.4 |
| 5.0 | 4 | 4.9 | 9.1 | −2.1 |
| 10.0 | 4 | 10.1 | 8.4 | 1.0 |
| 25.0 | 4 | 25.0 | 9.9 | 0.0 |
| 50.0 | 4 | 50.3 | 12.4 | 0.6 |
| 100.0 | 4 | 100.2 | 4.9 | 0.2 |

TABLE 9
Accuracy and precision of the method in
human plasma over 2 experiments

| | | Mean | | | | |
|---|---|---|---|---|---|---|
| Theo- | | calcu- | | Intra- | Inter- | |
| retical | | lated | | assay | assay | |
| conc. | | conc. | Accuracy¹ | precision² | precision³ | Recovery⁴ |
| (ng/ml) | n | (ng/ml) | (%) | (%) | (%) | (%) |
| 1.0 | 6 | 1.0 | 3.2 | 10.7 | 10.7 | 98.1 ± 3.8 |
| 25.0 | 6 | 25.3 | 1.3 | 11.6 | 13.1 | 98.6 ± 4.8 |
| 100.0 | 6 | 93.2 | −6.8 | 8.9 | 8.9 | 83.2 ± 5.2 |

¹Accuracy expressed with the mean bias obtained between theoretical and back-calculated concentrations.
²Intra-assay precision expressed with the coefficient of variation (CV) obtained over the 3 replicates in 2 experiments.
³Inter-assay precision expressed with the coefficient of variation (CV) obtained over the 3 replicates in 2 experiments.
⁴Recovery expressed by the mean ± SEM over 6 replicates.

### Efficiency of the Extraction Method

The efficiency of the extraction method was determined at three concentration levels corresponding to the QC samples. Recovery obtained was higher than 83.2±5.2% for CLF over the three concentration levels tested (mean±SEM, n=6 per concentration level, 3 concentration levels). Recovery obtained was higher than 52.3±9.5% for IS in our conditions of use (mean±SEM, n=18).

Recovery evaluated here includes both matrix effect and extraction yield of CLF and its IS from human plasma (Table 9).

### Stability of CLF in Human Blood

Stability of CLF in human blood and plasma was assayed over 3 distinct experiments. Samples whose CLF concentrations were higher than 100 ng/ml were 2-fold diluted in acetonitrile containing IS and re-injected.

First, CLF stability was studied in whole blood at 37° C. In this condition CLF was stable during 24 h. The percentage of compound remaining (by comparing with content at To) was at least 104.1%. The plasmatic concentrations measured were into the range 165.1-180.3 ng/ml while the concentration used was 100 ng/ml. This finding could be explained by the repartition of CLF in blood. Indeed, whole blood was spiked with 100 ng/ml of CLF. During plasma preparation, CLF was mainly distributed in the plasma fraction (corresponding to about the half-volume of the total sample). Results are shown in FIG. 7 and Table 10A.

Then, CLF stability was studied in plasma after a pre-incubation of whole blood at 37° C. for 30 min. After plasma preparation, CLF stability was studied 24 h at 4° C. and at room temperature. In this condition CLF was stable during 24 h. The percentage of compound remaining (by comparing with content at To) was at least 90.5% and 93.5% at room temperature and at 4° C., respectively. As previously observed, the plasmatic concentrations measured were in the range of 147.7-164.9 ng/ml whilst the concentration used was of 100 ng/ml. Results are shown in FIG. 7 and Table 10B.

CLF stability was studied directly in plasma during 24 h at 4° C. and at room temperature. In this condition CLF was stable for 24 h. The percentage of compound remaining (by comparing with content at To) was at least 83.7% and 88.2% at room temperature and at 4° C., respectively. The plasmatic concentrations measured were in the range of 85.3-101.9 ng/ml considering the concentration used (100 ng/ml). Results are shown in FIG. 7 and Table 10C.

In addition, CLF stability in plasma was studied in samples stored for 4 weeks at −70° C. Indeed, the percentage of compound remaining (by comparing with content at To) was 95.0% after 4 weeks at −70° C. The plasmatic concentrations measured were into the range 96.2-105.5 ng/ml considering the concentration used (100 ng/ml). Results are shown in FIG. 8 and Table 10D.

TABLE 10
Stability of CLF at 100 ng/ml in human blood. (A) Incubation of CLF
in whole blood at 37° C., (B) Pre-incubation of CLF in whole
blood at 37° C. during 30 minutes before preparation and
incubation of plasma at room temperature and 4° C.
(C) incubation of CLF in plasma at room temperature and 4° C.
and (D) storage of CLF in plasma at −70° C.
A

37° C.

| | | CLF | CLF |
|---|---|---|---|
| Time | | concentration¹ | remaining² |
| (hours) | n | (ng/ml) | (%) |
| 0.0 | 2 | 165.1 ± 2.6 | 100.0 |
| 0.5 | 2 | 171.9 ± 6.7 | 104.1 |
| 2.0 | 2 | 180.3 ± 7.4 | 109.2 |
| 24.0 | 2 | 173.8 ± 1.7 | 105.3 |

B

| | | | | Room temperature | |
|---|---|---|---|---|---|
| | | 4° C. | | (25 ± 2° C.) | |
| | | CLF | CLF | CLF | CLF re- |
| Time | | concentration¹ | remaining² | concentration¹ | maining² |
| (hours) | n | (ng/ml) | (%) | (ng/ml) | (%) |
| 0.0 | 2 | 163.3 ± 3.6 | 100.0 | 163.3 ± 3.6 | 100.0 |
| 0.5 | 2 | 155.4 ± 7.0 | 95.2 | 159.3 ± 0.6 | 97.6 |
| 2.0 | 2 | 164.9 ± 4.2 | 101.0 | 151.1 ± 0.8 | 92.5 |
| 24.0 | 2 | 152.7 ± 4.2 | 93.5 | 147.7 ± 3.7 | 90.5 |

C

| | | | | Room temperature | |
|---|---|---|---|---|---|
| | | 4° C. | | (25 ± 2° C.) | |
| | | CLF | CLF | CLF | CLF re- |
| Time | | concentration¹ | remaining² | concentration¹ | maining² |
| (hours) | n | (ng/ml) | (%) | (ng/ml) | (%) |
| 0.0 | 2 | 101.9 ± 4.6 | 100.0 | 101.9 ± 4.6 | 100.0 |
| 0.5 | 2 | 101.4 ± 3.5 | 99.5 | 85.3 ± 0.0 | 83.7 |
| 2.0 | 2 | 89.9 ± 3.4 | 88.2 | 93.2 ± 0.0 | 91.4 |
| 24.0 | 2 | 94.5 ± 3.6 | 92.7 | 100.7 ± 3.3 | 98.8 |

D

37° C.

| | | CLF | CLF |
|---|---|---|---|
| Time | | concentration¹ | remaining² |
| (days) | n | (ng/ml) | (%) |
| 0.0 | 2 | 101.2 ± 5.8 | 100.0 |
| 1.0 | 2 | 105.5 ± 2.5 | 104.3 |
| 3.0 | 2 | 104.7 ± 6.9 | 103.5 |
| 28.0 | 2 | 96.2 ± 1.4 | 95.0 |

¹Plasmatic concentration of CLF expressed by the mean ± SEM (n = 2).
²Percentage of CLF remaining obtained by comparing the mean concentrations at each incubation time with the mean concentration measured at To.

### Conclusions

The methodology described in Example 1 for quantification of CLF in plasma by HPLC-MS/MS was successfully validated in human plasma in terms of linearity, accuracy, intra- and inter-assay precision and extraction rate. Thus, stability of CLF in whole blood and plasma was demonstrated. CLF was found stable over 24 hours at 37° C. in whole human blood; Pretreatment of whole blood with CLF at 37° C. followed by collection of plasma and incubation at room temperature and at 4° C. showed CLF to be stable under these conditions. (up to 24 hours). CLF was also shown to be stable in plasma up to 4 weeks at −70° C. The relationship between concentration used for spiked whole blood and CLF concentrations quantified in plasma led us to conclude that CLF was mainly distributed in the plasmatic fraction recovered after centrifugation of whole blood.

### Example 3

### Immunoassay for the Detection of Cholyl-Lysyl-L-Fluorescein (CLF) in Plasma [Formerly Appendix C]

This report summarises a programme of experimental studies executed by NovAssay Ltd. to establish whether a prototype immunoassay could be established for the quantitative measurement of Cholyl-Lysyl-L-Fluorescein (CLF) in human blood serum or plasma, and to demonstrate the feasibility of achieving key assay performance claims with the prototype format.

CLF is a fluorescent analogue of the bile acid cholyl-lysine and has a number of existing and potential uses as an in vitro reagent for monitoring excretion to the bile canaliculi biliary tree imaging and others).

### Phase 1: Proof-of-Concept for Classical and Labelled Antibody Immunoassay Formats

Given the small size of the CLF molecule (molecular weight 992 Da), two competitive formats were adopted for initial evaluation, and appropriate reagents were purchased or prepared to allow their assessment.

### Classical Competitive Format

Streptavidin-coated microtitre plate Biotinylated anti-fluorescein antibody, “BJ/C” (NovAssay, using HyTest mouse monoclonal anti-fluorescein, cat code 5F3 2A3 (conjugate ref. DV038/182-1) or AbD Serotec sheep polyclonal anti-fluorescein, cat code 640001 (ref. DV038/182-2) is bound to the plate CLF from the sample competes with CLF-horseradish peroxidase, “CJ/C” (CLF-HRP; NovAssay, DV047/14) for the biotinylated anti-fluorescein antibody Bound CLF-HRP is measured colorimetrically using tetramethylbenzidine (TMB) substrate.

Twelve variations of this format were used to measure 0, 20 and 2000 ng/ml samples of CLF (BD 451041) diluted in buffer (see Table 12).

TABLE 12

| | MAB | MAB | MAB | polyclonal | polyclonal | polyclonal |
|---|---|---|---|---|---|---|
| | Assay 1 | Assay 2 | Assay 3 | Assay 4 | Assay 5 | Assay 6 |
| BJ/C Lot | DV038/182/1 | DV038/182/1 | DV038/182/1 | DV038/182/2 | DV038/182/2 | DV038/182/2 |
| BJ conc (ng/ml) | 10 | 100 | 1000 | 10 | 100 | 1000 |
| CJ/C Lot | DV047/14 | DV047/14 | DV047/14 | DV047/14 | DV047/14 | DV047/14 |
| CJ conc (ng/ml) | 20 | 20 | 20 | 20 | 20 | 20 |
| | MAB | MAB | MAB | polyclonal | polyclonal | polyclonal |
| | Assay 7 | Assay 8 | Assay 9 | Assay 10 | Assay 11 | Assay 12 |
| BJ/C Lot | DV038/182/1 | DV038/182/1 | DV038/182/1 | DV038/182/2 | DV038/182/2 | DV038/182/2 |
| BJ conc (ng/ml) | 10 | 100 | 1000 | 10 | 100 | 1000 |
| CJ/C Lot | DV047/14 | DV047/14 | DV047/14 | DV047/14 | DV047/14 | DV047/14 |
| CJ conc (ng/ml) | 100 | 100 | 100 | 100 | 100 | 100 |

The results demonstrated that in a number of these variations the assay could readily distinguish between the three CLF levels (see FIG. 9).

It was therefore concluded that the classical competitive format had the potential to yield an effective CLF assay.

### Labelled-Antibody Competitive Format

Streptavidin-coated microtitre plate
Biotin-fluorescein (Sigma B9431) is bound to the plate
CLF from the sample competes with bound biotin-fluorescein for an anti-fluorescein-HRP conjugate (NovAssay, using HyTest mouse monoclonal anti-fluorescein, cat code 5F3 2A3 (conjugate ref. DV047/17-3)
Bound anti-fluorescein-HRP is measured colorimetrically using tetramethylbenzidine (TMB) substrate.

Twelve variations of this format were used to measure 0 and 20 ng/ml samples of CLF diluted in buffer (see table 13).

TABLE 13

| | Assay 1 | Assay 2 | Assay 3 | Assay 4 | Assay 5 | Assay 6 |
|---|---|---|---|---|---|---|
| BJ/C Lot | FTC-biotin | FTC-biotin | FTC-biotin | FTC-biotin | FTC-biotin | FTC-biotin |
| BJ conc (ng/ml) | 10 | 100 | 1000 | 10000 | 10 | 100 |
| CJ/C Lot | DV047/17-3 | DV047/17-3 | DV047/17-3 | DV047/17-3 | DV047/17-3 | DV047/17-3 |
| CJ conc (ng/ml) | 10 | 10 | 10 | 10 | 100 | 100 |
| | Assay 7 | Assay 8 | Assay 9 | Assay 10 | Assay 11 | Assay 12 |
| BJ/C Lot | FTC-biotin | FTC-biotin | FTC-biotin | FTC-biotin | FTC-biotin | FTC-biotin |
| BJ conc (ng/ml) | 1000 | 10000 | 10 | 100 | 1000 | 10000 |
| CJ/C Lot | DV047/173 | DV047/17-3 | DV047/17-3 | DV047/17-3 | DV047/17-3 | DV047/17-3 |
| CJ conc (ng/ml) | 100 | 100 | 1000 | 1000 | 1000 | 1000 |

The results were less encouraging than those from the classical format, but demonstrated that in Variations 5 and 9 the assay could adequately distinguish between the two CLF levels (see FIG. 10).

It was therefore concluded that the labelled-antibody competitive format also had the potential to yield an effective CLF assay.

### Phase 2a: Optimisation of Classical and Labelled Antibody Immunoassay Formats Standard Curves

Following on from the Phase 1 proof-of-concept results, full sets of CLF standards covering a range of 0-1000 ng/ml were prepared (a) in buffer to emulate Phase 1 conditions, and (b) in horse serum, as a matrix more closely related to the actual intended sample type and therefore considered more appropriate for use in the final assay format. Both assay formats were re-optimised from the Phase 1 conditions, with the result that both yielded good standard curves.

In both formats there was virtually no difference in performance between the buffer and the horse serum standards.

### Recovery of Spiked CLF

Given the success of achieving workable standard curves in both formats, 100 ng/ml of CLF was spiked into a number of individual human serum samples, and the percentage CLF recovery calculated (e.g. 50 ng/ml=50%). This experiment was intended to confirm that CLF could be quantitatively recovered from human serum, and that there was no sample-to-sample variation in this recovery (ideal result=100% for all samples; individual sample recoveries of ±20% generally considered acceptable). See FIG. 11.

The results indicated two problems: a generally low level of recovery (all results ≦60%), and significant sample-to-sample variability (e.g. 0% recovery in the classical format for sample #6 vs. 60% for sample #3).

These results suggest that (a) CLF binds to serum proteins and is therefore “lost” from the assay, and (b) that the amount of CLF lost varies from one sample to another. This recovery performance is unacceptable in terms of generating a workable assay.

The particularly wide variability of the classical format—especially the complete loss of measurable CLF from sample #6—suggested that the labelled-antibody format might offer better prospects for improvement in this respect.

Regarding the generally low recovery, it was surmised that there was a higher level of binding to human serum than to horse serum (used in the standard curve), therefore (for the labelled-antibody format only), the spiked recovery experiment was repeated using a standard curve generated using human serum-based standards (see FIG. 12).

This was successful in shifting the mean recovery to ˜100%. However, an unacceptably wide sample-to-sample variation was still observed (˜70-180%), therefore it was concluded that a blocker approach would be required, in which a chemical agent is used to displace the analyte from the binding proteins, so that it can be recovered effectively in the assay. Initial studies using various known assay blockers in the labelled-antibody format (data not shown) indicated that 8-anilino-1-naphthalene sulfonic acid (ANS) and to a lesser extent deoxycholic acid showed promise for this purpose. ANS was therefore evaluated in more detail in this format.

### Effect of ANS on CLF Recovery in the Labelled-Antibody Format

The following study was executed to assess the recovery of CLF following spiking into human serum in assays, in the presence and absence of ANS.

25 μl of standard (in buffer) was added to streptavidin coated wells, followed by 100 ul of 20 ng/ml biotin-fluorescein (one formulation containing 0.2% ANS, the other without) and 100 μl of anti-fluorescein HRP (100 ng/ml). After 30 minutes incubation (RT/static) the wells were washed and 100 μl of SureBlue Reserve TMB substrate added. After a further 10 minutes incubation, 100 μl of 1M HCl was added and the colour intensity measured at 450 nm.

3 plasma samples (Lithium heparin) were spiked with CLF at 125 and 500 ug/ml; these concentrations (the expected values) are noted in the relevant sample IDs.

Table 14 and FIG. 13 show the standard curves obtained in the 2 assays (with/without ANS)

TABLE 14

| | OD450 | OD450 |
|---|---|---|
| | without | with |
| | ANS | ANS |
| 1000 | 1.0092 | 0.2382 |
| 500 | 1.5039 | 0.4304 |
| 250 | 1.8688 | 0.7230 |
| 125 | 2.1779 | 1.1028 |
| 62.5 | 2.5350 | 1.9869 |
| 31.25 | 2.5879 | 2.2317 |
| 15.625 | 2.7834 | 2.9673 |
| 0 | 2.8581 | 3.9688 |

Table 15 shows the CLF concentrations obtained in the two assays (with/without ANS). The data are also shown in FIG. 14.

TABLE 15

| | | ng/ml | ng/ml |
|---|---|---|---|
| sample | ID in | without | with |
| ID* | graph | ANS | ANS |
| #5879, | 1.1 | 112 | 562 |
| 500 | | | |
| #5879, | 1.2 | 3.6 | 168 |
| 125 | | | |
| #5878, | 2.1 | 53.3 | 444 |
| 500 | | | |
| #5878, | 2.2 | 0.4 | 128 |
| 125 | | | |
| #5877, | 3.1 | 55 | 481 |
| 500 | | | |
| #5877, | 3.2 | 3.6 | 148 |
| 125 | | | |

Table 16 shows a plot of the % recovery (observed/expected) of spiked CLF in the two assays. The data are also shown in FIG. 15.

TABLE 16

| | ID in | without | with |
|---|---|---|---|
| sample ID | graph | ANS | ANS |
| #5879, | 1.1 | 22% | 112% |
| 500 | | | |
| #5879, | 1.2 | 3% | 134% |
| 125 | | | |
| #5878, | 2.1 | 11% | 89% |
| 500 | | | |
| #5878, | 2.2 | 0% | 102% |
| 125 | | | |
| #5877, | 3.1 | 11% | 96% |
| 500 | | | |
| #5877, | 3.2 | 3% | 118% |
| 125 | | | |

The results clearly show that ANS is effective in displacing CLF bound to serum/plasma proteins. In the absence of ANS the recovery is very low, especially at low CLF concentrations.

### Effect of ANS and Deoxycholic Acid on CLF Recovery in the Classical Format

The recovery performance of the classical competitive format was considered to be inferior based on initial results (see “Effect of ANS on CLF Recovery in the Labelled-Antibody Format”). However, given the discovery that blockers such as ANS and deoxycholic acid are able to displace CLF from serum proteins, an evaluation of these blockers in the classical format was considered worthwhile.

10 μl of standard was added to streptavidin coated wells, followed by 100 μl of fluorescein-HRP (100 ng/ml) and 100 μl of Mab-biotin (100 ng/ml). After 30 (or 60 minutes, see below) minutes incubation (RT/static) the wells were washed and 100 μl of substrate added. After a further 10 minutes incubation, 100 μl of 1M HCl was added and the colour intensity measured at 450 nm.

Several assays were carried out using different biotin-Mab reagent formulations. These are described below:
Mab-biotin at 100 ng/ml containing 0.2% ANS. Incubation time was 30 minutes.
Mab-biotin at 100 ng/ml containing 5 mM Deoxycholic acid. Incubation time was 30 minutes.
Mab-biotin at 100 ng/ml containing 1.25 mM Deoxycholic acid. Incubation time was 60 minutes.
Mab-biotin at 100 ng/ml containing 0.05% ANS. Incubation time was 60 minutes.

Control assays (with no blocker in the biotin-MAB reagent) were also run (only standards assayed).

In the assays that used formulations that contained the blockers, 16 individual donor samples (serum) were spiked with 100 ng/ml CLF. These samples (and unspiked samples) were assayed alongside freshly made buffer-based standards.

FIGS. 16 to 18 shows the standard curves obtained in the control formulation (no blocker) and the formulations containing either the 0.2% ANS or 5 mM Deoxycholic acid (incubation was 30 minutes).

The results show that addition of the blockers resulted in reduced signals and much steeper curves. The steepness of the curves resulted in a reduced assay range. Thus, in the interpolation of the unknown signals to dose, the standard curve range was reduced to 125 ng/ml.

The data below show the concentrations obtained in the CLF spiked samples (the concentrations in the unspiked samples were not significantly different from zero).

Table 17 shows the calculated doses (and % recovery) in the formulation containing 5 mM Deoxycholic acid.

TABLE 17

| | | % |
|---|---|---|
| ID | ng/ml | recovery |
| 237 | 93 | 93% |
| 182 | 88 | 88% |
| 170 | 96 | 96% |
| 169 | 96 | 96% |
| 231 | 82 | 82% |
| 180 | 90 | 90% |
| 178 | 80 | 80% |
| 236 | 79 | 79% |
| 225 | 97 | 97% |
| 219 | 20 | 20% |
| 221 | 16 | 16% |
| 230 | 96 | 96% |
| 229 | 86 | 86% |
| 238 | 87 | 87% |
| 240 | 82 | 82% |
| 223 | 81 | 81% |
| mean | | 79% |
| recovery | | |

Table 18 shows the calculated doses (and % recovery) in the formulation containing 0.2% ANS.

TABLE 18

| | | % |
|---|---|---|
| ID | ng/ml | recovery |
| 237 | 107 | 107% |
| 182 | 108 | 108% |
| 170 | 116 | 116% |
| 169 | 117 | 117% |
| 231 | 112 | 112% |
| 180 | 106 | 106% |
| 178 | 104 | 104% |
| 236 | 109 | 109% |
| 225 | 99 | 99% |
| 219 | 119 | 119% |
| 221 | 116 | 116% |
| 230 | 86 | 86% |
| 229 | 105 | 105% |
| 238 | 115 | 115% |
| 240 | 104 | 104% |
| 223 | 102 | 102% |
| mean | | 108% |

In the presence of the blockers (even the reduced concentrations) the signals were reduced with the curves becoming steeper. The steeper curves in effect reduced the assay range (up to 500 ng/ml with the 1.25 mM Deoxycholic acid and 250 ng/ml with the 0.05% ANS).

Table 19 shows the calculated doses (and % recovery) in the formulation containing 1.25 mM Deoxycholic acid.

TABLE 19

| | | % |
|---|---|---|
| ID | ng/ml | recovery |
| 237 | 77 | 77% |
| 182 | 41 | 41% |
| 170 | 64 | 64% |
| 169 | 54 | 54% |
| 231 | 33 | 33% |
| 180 | 26 | 26% |
| 178 | 45 | 45% |
| 236 | 54 | 54% |
| 225 | 25 | 25% |
| 219 | 26 | 26% |
| 221 | 51 | 51% |
| 230 | 64 | 64% |
| 229 | 40 | 40% |
| 238 | 53 | 53% |
| 240 | 42 | 42% |
| 223 | 40 | 40% |
| Mean % | | 46% |
| recovery | | |

Table 20 shows the calculated doses (and % recovery) in the formulation containing 0.05% ANS.

TABLE 20

| | | % |
|---|---|---|
| | ng/ml | recovery |
| 237 | 112 | 112% |
| 182 | 127 | 127% |
| 170 | 119 | 119% |
| 169 | 97 | 97% |
| 231 | 141 | 141% |
| 180 | 173 | 173% |
| 178 | 78 | 78% |
| 236 | 81 | 81% |
| 225 | 138 | 138% |
| 219 | 157 | 157% |
| 221 | 104 | 104% |
| 230 | 112 | 112% |
| 229 | 94 | 94% |
| 238 | 108 | 108% |
| 240 | 72 | 72% |
| 223 | 81 | 81% |
| Mean % | | 112% |
| recovery | | |

The results presented show that addition of 5 mM Deoxycholic acid in the formulation of the “classical” competitive CLF assay resulted in reduced signals and steepening of the curve which in effect reduced the assay range (to 125 ng/ml).

Although mean % recovery of added CLF in 16 individual sera was marginally acceptable at 79%, unacceptable recoveries were observed in 2 sera (only 16% and 20% obtained).

With 0.2% ANS the curve was even steeper than was seen with Deoxycholic acid but the mean recovery was good (at 108%). In an attempt to improve the curve shape (i.e. have the range up to 1000 ng/ml) and increase the signal, the amount of blocker used was quartered and the assay incubation time increased from 30 minutes to 60 minutes. The results obtained suggested that the signals, and assay ranges increased (calculated range with the Deoxycholic acid was 500 ng/ml and it was 250 ng/ml with the 0.05% ANS), but the CLF recoveries were poor. The average CLF recovery with the 1.25 mM Deoxycholic acid was only 46% and whilst it was higher with ANS (112%) the sample to sample variability (minimum of 72%, maximum 173%) resulted in unacceptable performance.

On the basis of these results it was decided not to pursue the classical competitive assay format any further but to concentrate on the labelled-antibody format which demonstrated superior results.

Optimisation of Precision of the Labelled-Antibody Format with ANS

With the issue of variable binding to serum proteins fixed in the labelled-antibody format by the incorporation of ANS as a blocker, the next objective was to determine whether the precision of the resulting assay was acceptable.

10 μl of standard (in buffer) was added to streptavidin coated wells, followed by 100 μl of biotin-fluorescein (10 ng/ml containing 0.2% ANS) and 100 μl of anti-fluorescein HRP (100 ng/ml). After 30 minutes incubation (RT/static) the wells were washed and 100 μl of SureBlue Reserve added. After a further 10 minutes incubation, 100 ul of 1M HCl was added and the colour intensity measured at 450 nm.

3 individual donor samples (heparin plasmas) were spiked with 1000 ng/ml CLF. Each of these spiked samples was then serially diluted with the respective unspiked samples. The expected concentrations were thus 1000, 500, 250, 125, 62.5, 31.25, and 15.6 ng/ml. Each of these samples was then assayed in 8 replicates in order to obtain the intra-assay CV at each dose. The zero standard was also assayed (8 reps) to determine the analytical sensitivity. Analytical sensitivity was calculated by the interpolating into dose the OD corresponding to the mean zero signal minus 2SDs (calculated from the unspiked individual plasma signal).

Other experiments were carried out using an increased sample volume and biotin load. The details of these experiments will be shown with the results.

Tables 21 to 23 summarise the intra-assay precision obtained in the 3 individual plasmas used.

TABLE 21

| CLF | plasma 5877 | | | | | | |
|---|---|---|---|---|---|---|---|
| (ng/ml) | 1000 | 500 | 250 | 125 | 62.5 | 31.25 | 15.6 |
| Rep 1 | 895 | 569 | 225 | 122 | 72 | 46 | 27 |
| Rep 2 | 936 | 438 | 222 | 115 | 67 | 41 | 23 |
| Rep 3 | 925 | 458 | 212 | 123 | 98 | 40 | 30 |
| Rep 4 | 871 | 452 | 232 | 117 | 54 | 36 | 30 |
| Rep 5 | 1149 | 439 | 239 | 124 | 61 | 31 | 40 |
| Rep 6 | 1015 | 400 | 225 | 111 | 66 | 42 | 25 |
| Rep 7 | 786 | 400 | 224 | 122 | 66 | 45 | 27 |
| Rep 8 | 910 | 410 | 221 | 123 | 91 | 35 | 26 |
| mean | 936 | 446 | 225 | 120 | 72 | 40 | 29 |
| SD | 107.6 | 54.6 | 7.8 | 4.8 | 15.1 | 5.4 | 5.3 |
| % CV | 11.5% | 12.3% | 3.5% | 4.0% | 21.0% | 13.6% | 18.6% |
| Sensitivity | 3.6 ng/ml | | | | | | |

TABLE 22

| CLF | plasma 5878 | | | | | | |
|---|---|---|---|---|---|---|---|
| (ng/ml) | 1000 | 500 | 250 | 125 | 62.5 | 31.25 | 15.6 |
| Rep 1 | 1184 | 603 | 301 | 145 | 47 | 31 | 14.3 |
| Rep 2 | 1032 | 608 | 268 | 70 | 94 | 43 | 10.7 |
| Rep 3 | 1200 | 591 | 266 | 88 | 87 | 38 | 9.5 |
| Rep 4 | 1120 | 473 | 337 | 151 | 79 | 52 | 14.2 |
| Rep 5 | 1143 | 559 | 241 | 124 | 82 | 31 | 10.1 |
| Rep 6 | 978 | 501 | 266 | 107 | 72 | 35 | 8.0 |
| Rep 7 | 1040 | 517 | 269 | 117 | 64 | 27 | 6.8 |
| Rep 8 | 1065 | 496 | 238 | 153 | 64 | 31 | 11.4 |
| Mean | 1095 | 543 | 273 | 119 | 74 | 36 | 11 |
| SD | 78.9 | 53.3 | 32.2 | 30.0 | 15.1 | 8.3 | 2.7 |
| % CV | 7.2% | 9.8% | 11.8% | 25.1% | 20.4% | 23.1% | 25.1% |
| Sensitivity | 4.1 ng/ml | | | | | | |

TABLE 23

| CLF | plasma 6879 | | | | | | |
|---|---|---|---|---|---|---|---|
| (ng/ml) | 1000 | 500 | 250 | 125 | 62.5 | 31.25 | 15.6 |
| Rep 1 | 1127 | 457 | 283 | 124 | 73 | 44 | 28 |
| Rep 2 | 1133 | 477 | 265 | 126 | 64 | 40 | 26 |
| Rep 3 | 1007 | 448 | 261 | 118 | 70 | 46 | 21 |
| Rep 4 | 1043 | [1284] | 281 | 151 | 78 | 48 | 20 |
| Rep 5 | 993 | 563 | 249 | 135 | 46 | 41 | 19 |
| Rep 6 | 985 | 477 | 245 | 126 | 75 | 46 | 25 |
| Rep 7 | 951 | 479 | 274 | 93 | 65 | 38 | 13 |
| Rep 8 | 1029 | 463 | 242 | 129 | 58 | 46 | 19 |
| Mean | 1033 | 481 | 262 | 125 | 66 | 44 | 21 |
| SD | 65.6 | 38.3 | 15.9 | 16.4 | 10.4 | 3.4 | 4.7 |
| % CV | 6.3% | 8.0% | 6.1% | 13.1% | 15.7% | 7.8% | 22.0% |
| Sensitivity | 6.8 ng/ml | | | | | | |

The precision appeared to be unacceptable with % CVs often exceeding 10%. The most likely cause of imprecision was considered to be the small sample size (10 μl). For this reason another assay was carried out using a 25 μl sample volume.

The results are summarised in Table 24.

TABLE 24

| CLF | Sample 7877 | | | | | | |
|---|---|---|---|---|---|---|---|
| (ng/ml) | 1000 | 500 | 250 | 125 | 62.5 | 31.25 | 15.6 |
| Rep 1 | 873 | 484 | 221 | 122 | 64 | 30 | 15 |
| Rep 2 | 903 | 467 | 226 | 114 | 64 | 32 | 17 |
| Rep 3 | 861 | 474 | 226 | 132 | 63 | 33 | 18 |
| Rep 4 | 911 | 449 | 210 | 128 | 64 | 34 | 20 |
| Rep 5 | 867 | 436 | 229 | 124 | 63 | 33 | 19 |
| Rep 6 | 840 | 450 | 211 | 119 | 60 | 34 | 20 |
| Rep 7 | 837 | 452 | 216 | 111 | 61 | 33 | 17 |
| Rep 8 | 784 | 415 | 219 | 111 | 57 | 31 | 18 |
| Mean | 859 | 453 | 220 | 120 | 62 | 32 | 18 |
| SD | 40.2 | 21.9 | 7.2 | 7.9 | 2.6 | 1.4 | 1.6 |
| % CV | 4.7% | 4.8% | 3.3% | 6.6% | 4.2% | 4.3% | 9.1% |
| Sensitivity | 2. ng/ml | | | | | | |

It is clear from these data (Table 24) that the precision is greatly improved when the sample size is increased to 25 μl (from 10 μl). However, as shown in Table 25 and FIG. 19, the assay is too steep which could prevent achieving the necessary assay range of 1000 ng/ml.

TABLE 25

| ng/ml | OD450 |
|---|---|
| 1000 | 0.119 |
| 500 | 0.200 |
| 250 | 0.330 |
| 125 | 0.578 |
| 62.5 | 0.963 |
| 31.25 | 1.498 |
| 15.625 | 1.899 |
| 0 | 4.181 |

Table 26 shows the raw data of the 15 and 20 ng/ml biotin-MAB experiment.

TABLE 26

| | 20 ug/ml | 15 ug/ml |
|---|---|---|
| 1000 | 0.2005 | 0.1524 |
| 500 | 0.3283 | 0.3208 |
| 250 | 0.6162 | 0.5704 |
| 125 | 0.9912 | 0.9506 |
| 62.5 | 1.5031 | 1.4114 |
| 31.25 | 2.0410 | 2.0589 |
| 15.625 | 2.6054 | 2.3962 |
| 0 | 4.0291 | 3.8248 |

The results presented suggested that precision using the existing conditions (10 μl sample, 10 ng/ml biotin-MAB) were not acceptable. A significant improvement to the precision was achieved by increasing the sample volume from 10 μl to 25 μl, but the curve shape suggested that a small degree of desensitisation was required to assure the required assay range (1000 ng/ml).

Increasing the biotin-MAB concentration gave a better curve (the OD at 1000 ng/ml was almost double of the corresponding OD obtained with the 10 ng/ml biotin-MAB formulation). This change was therefore adopted.

Conclusions from Optimisation Studies

The studies described in Phase 2a (above) resulted in a labelled-antibody competitive immunoassay format which gave an acceptable range, precision and recovery of CLF from human serum and plasma samples.

It was concluded that this format met the key design goals for Phase 2, and it was therefore adopted for the performance verification studies (in Phase 2b below) and the evaluation of HPLC samples (below).

### Optimised Assay Format

These conditions were used for all assays described in Phase 2a (above) and Phase 2b (below). See Appendix B and C for buffer and conjugate formulations respectively:
Add 25 μl/well of sample or standard (0-1000 ng/ml CLF in Buffer #24) to the wells of a streptavidin-coated microtitre plate (NovAssay, F2SAC).
Add 100 μl/well of fluorescein-biotin (Sigma B9431) at 20 ng/ml in Buffer #24+0.2% w/v ANS (Sigma 10417).
Add 100 μl/well of anti-fluorescein HRP (NovAssay, DV047/17-3) at 100 ng/ml in Buffer #24.
Incubate the plate for 30 minutes at room temperature.
Wash the plate 3 times with 800 μl/well Buffer #29 and tap the plate dry.
Add 100 μl/well of SureBlue Reserve TMB Substrate (KPL) and incubate the plate for 10 minutes at room temperature.
Stop the substrate reaction by adding 100 μl/well of 1M HCl and read the absorbance at 450 nm wavelength (ThermoFisher Scientific, Multiskan FC).

### Phase 2B: Demonstration of Feasibility of Key Assay Performance Characteristics

### Inter-Assay Precision

In order to assess the inter-assay precision of the CLF assay, six assays were run. Each assay consisted of freshly made buffer standards and “QC controls”. The controls were prepared by spiking CLF to a human plasma pool at 3 concentrations (QC1=500 ng/ml, QC2=125 ng/ml and QC3=31.25 ng/ml).

A summary of the results is shown on Table 27.

TABLE 27

| | Assay | Assay | Assay | Assay | Assay | Assay | | Std | |
|---|---|---|---|---|---|---|---|---|---|
| | 1 | 2 | 3 | 4 | 5 | 6 | Mean | Dev | % CV |
| QC1 | 363 | 400 | 398 | 428 | 452 | 462 | 417 | 37.2 | 8.9% |
| (500 ng/ml) | | | | | | | | | |
| QC2 | 112 | 114 | 127 | 119 | 117 | 117 | 118 | 5.2 | 4.5% |
| (125 ng/ml) | | | | | | | | | |
| QC3 | 32 | 36 | 31 | 37 | 36 | 36 | 35 | 2.5 | 9.4% |
| (31.5 ng/ml) | | | | | | | | | |

The inter-assay CV (across 6 assays and 3 CLF concentrations) was <10% (range was 4.5% to 9.4%).

### Intra-Assay Precision, Sensitivity and Dilution Recovery of CLF Added to Different Plasmas

Five individual donor samples (Heparin plasmas) were spiked with 1000 ng/ml CLF (CLF dissolved in aqueous/ethanol solution (see Example 4). Each of these spiked samples was then serially diluted with the respective unspiked samples. The expected concentrations were thus 1,000, 500, 250, 125, 62.5, 31.25, and 15.6 ng/ml. Each of these samples was then assayed in 8 replicates in order to obtain the intra-assay CV at each dose. The zero standard was also assayed (8 reps) to determine the analytical sensitivity. Analytical sensitivity was calculated by the interpolating into dose the OD corresponding to the mean zero signal minus 2SDs (calculated from the unspiked individual plasma signal).

For recovery two different calculations were performed. One involved comparing the observed concentration with expected. This gives an estimate of the recovery after spiking. The second calculation involves multiplying the observed concentration by the dilution factor and comparing the results with the expected results for example, 1000 ng/ml is added to a plasma sample and the detected concentration is determined at 900 ng/ml. The “spiking” recovery will thus be 90%. If this sample is diluted 1 in 2, then the expected concentration will be 450 ng/ml (since the neat sample had a concentration of 900 ng/ml). If the 1 in 2 dilution gives a value of 400 ng/ml then the dilutional recovery will be 400/450=89%.

A summary of the intra-assay precision, sensitivity, spiking and dilution recovery for each plasma is shown on tables 28 to 33.

TABLE 28

| | plasma 5878 | | | | |
|---|---|---|---|---|---|
| | | observed | | “spiking” | dilutional |
| dilution factor | expected | mean | % CV | recovery | recovery |
| 1 | 1000 | 963 | 2.6 | 96% | |
| 2 | 500 | 461 | 5.8 | 92% | 96% |
| 4 | 250 | 227 | 6.3 | 91% | 94% |
| 8 | 125 | 113 | 6.7 | 90% | 94% |
| 16 | 62.5 | 62 | 7.6 | 100% | 104% |
| 32 | 31.25 | 36 | 13.4 | 114% | 118% |
| 64 | 15.6 | 22 | 22.2 | 141% | 147% |
| | 0 | 5 | 23.4 | | |

sensitivity 2.8 ng/ml

TABLE 29

| | plasma 5881 | | | | |
|---|---|---|---|---|---|
| dilution | | observed | | “spiking” | dilutional |
| factor | expected | mean | % CV | recovery | recovery |
| 1 | 1000 | 878 | 6 | 88% | |
| 2 | 500 | 424 | 7 | 85% | 97% |
| 4 | 250 | 204 | 6 | 82% | 93% |
| 8 | 125 | 109 | 3 | 87% | 99% |
| 16 | 62.5 | 60 | 9 | 96% | 109% |
| 32 | 31.25 | 35 | 5 | 113% | 129% |
| 64 | 15.6 | 18 | 10 | 118% | 134% |
| | 0 | 5 | 18 | | |

sensitivity 2.8 ng/ml

TABLE 30

| | plasma 5883 | | | | |
|---|---|---|---|---|---|
| dilution | | observed | | “spiking” | dilutional |
| factor | expected | mean | % CV | recovery | recovery |
| 1 | 1000 | 1022 | 3.3 | 102% | |
| 2 | 500 | 478 | 6.3 | 96% | 94% |
| 4 | 250 | 228 | 3.7 | 91% | 89% |
| 8 | 125 | 113 | 4.8 | 91% | 89% |
| 16 | 62.5 | 56 | 11.5 | 89% | 87% |
| 32 | 31.25 | 32 | 7.6 | 102% | 100% |
| 64 | 15.6 | 20 | 6.4 | 126% | 123% |
| | 0 | 5 | 14.0 | | |

sensitivity 3 ng/ml

TABLE 31

| | plasma 5884 | | | | |
|---|---|---|---|---|---|
| | | observed | | “spiking” | dilutional |
| dilution factor | expected | mean | % CV | recovery | recovery |
| 1 | 1000 | 841 | 6.9 | 84% | |
| 2 | 500 | 478 | 6.3 | 96% | 114% |
| 4 | 250 | 228 | 3.7 | 91% | 109% |
| 8 | 125 | 113 | 4.8 | 91% | 108% |
| 16 | 62.5 | 56 | 11.5 | 89% | 106% |
| 32 | 31.25 | 32 | 7.6 | 102% | 121% |
| 64 | 15.6 | 20 | 6.4 | 126% | 150% |
| | 0 | 5 | 14.0 | | |

sensitivity 3.2 ng/ml

TABLE 32

| | plasma 5885 | | | | |
|---|---|---|---|---|---|
| | | observed | | “spiking” | dilutional |
| dilution factor | expected | mean | % CV | recovery | recovery |
| 1 | 1000 | 872 | 10.7 | 87% | |
| 2 | 500 | 425 | 7.6 | 85% | 97% |
| 4 | 250 | 217 | 8.8 | 87% | 99% |
| 8 | 125 | 118 | 7.9 | 94% | 108% |
| 16 | 62.5 | 71 | 6.6 | 114% | 131% |
| 32 | 31.25 | 33 | 13.7 | 105% | 121% |
| 64 | 15.6 | 17 | 22.5 | 107% | 123% |
| | 0 | 5 | 147.6 | | |

sensitivity 3.8 ng/ml

TABLE 33

| | plasma 5876 | | | | |
|---|---|---|---|---|---|
| dilution | | observed | | “spiking” | dilutional |
| factor | expected | mean | % CV | recovery | recovery |
| 1 | 1000 | 815 | 6 | 82% | |
| 2 | 500 | 350 | 8 | 70% | 86% |
| 4 | 250 | 230 | 7 | 92% | 113% |
| 8 | 125 | 110 | 15 | 88% | 108% |
| 16 | 62.5 | 67 | 8 | 107% | 132% |
| 32 | 31.25 | 39 | 7 | 124% | 152% |
| 64 | 15.6 | 22 | 15 | 138% | 169% |
| | 0 | 3 | 69 | | |

sensitivity 3.8 ng/ml

Table 34 shows the individual spiking recovery data and the average recovery of each plasma and overall recovery (across the 6 plasmas).

TABLE 34
Spiking recovery

| expected | #5878 | #5881 | #5883 | #5884 | #5885 | #5876 | mean |
|---|---|---|---|---|---|---|---|
| 1000 | 96% | 88% | 102% | 84% | 87% | 82% | 90% |
| 500 | 92% | 85% | 96% | 96% | 85% | 70% | 87% |
| 250 | 91% | 82% | 91% | 91% | 87% | 92% | 89% |
| 125 | 90% | 87% | 91% | 91% | 94% | 88% | 90% |
| 62.5 | 100% | 96% | 89% | 89% | 114% | 107% | 99% |
| 31.25 | 114% | 113% | 102% | 102% | 105% | 124% | 110% |
| 15.6 | 141% | 118% | 126% | 126% | 107% | 138% | 126% |
| mean recovery | 104% | 95% | 100% | 97% | 97% | 100% | |
| Overall mean | | | | | | | 99% |

Table 35 shows the individual dilution recovery data and the average recovery of each plasma and overall recovery (across the 6 plasmas).

TABLE 35
Dilution recovery

| expected | #5878 | #5881 | #5883 | #5884 | #5885 | #5876 | mean |
|---|---|---|---|---|---|---|---|
| 1000 | | | | | | | |
| 500 | 96% | 97% | 94% | 114% | 97% | 86% | 97% |
| 250 | 94% | 93% | 89% | 109% | 99% | 113% | 100% |
| 125 | 94% | 99% | 89% | 108% | 108% | 108% | 101% |
| 62.5 | 104% | 109% | 87% | 106% | 131% | 132% | 111% |
| 31.25 | 118% | 129% | l00% | 121% | 121% | 152% | 123% |
| 15.6 | 147% | 134% | 123% | 150% | 123% | 169% | 141% |
| mean recovery | 109% | 110% | 97% | 118% | 113% | 127% | |
| Overall mean | | | | | | | 112% |

Table 36 shows the intra-assay precision and sensitivity in each of the assay plates (and the overall means).

TABLE 36
Intra-assay Precision (% CV) and sensitivity

| expected | #5878 | #5881 | #5883 | #5884 | #5885 | #5876 | mean |
|---|---|---|---|---|---|---|---|
| 1000 | 2.6 | 6 | 3.3 | 6.9 | 10.7 | 5.9 | 6 |
| 500 | 5.8 | 7 | 6.3 | 6.3 | 7.6 | 8.5 | 7 |
| 250 | 6.3 | 6 | 3.7 | 3.7 | 8.8 | 7.2 | 6 |
| 125 | 6.7 | 3 | 4.8 | 4.8 | 7.9 | 15.0 | 7 |
| 62.5 | 7.6 | 9 | 11.5 | 11.5 | 6.6 | 8.5 | 9 |
| 31.25 | 13.4 | 5 | 7.6 | 7.6 | 13.7 | 6.8 | 9 |
| 15.6 | 22.2 | 10 | 6.4 | 6.4 | 22.5 | 15.4 | 14 |
| sensitivity | 2.8 | 3 | 3.3 | 3.2 | 3.8 | 3.8 | |
| Overall precision | | | | | | | 8% |
| Overall sensitivity | 3.3 ng/ml | | | | | | |

The results presented show that the assay is able to deliver a <10% intra-assay precision across a range of 31.25 to 1000 ng/ml. The precision at lower doses e.g. 15.6 ng/ml was higher than 10% (at 14%) which is likely to be acceptable bearing in mind that the required sensitivity is 20 ng/ml. The actual sensitivity obtained was 3.3 ng/ml.

The spiking recovery data (mean % recovery of 99%) suggest that the assay is able to detect CLF added to plasma over the assay range. The mean dilution recovery was 112% over a concentration range of 15.6 to 1000 ng/ml (106% over a range of 31 to 1000 ng/ml).

The performance at this stage of development (feasibility stage) is therefore deemed acceptable. More work will be required in the next stage to challenge the performance using several reagents (conjugates/formulations), time (age of reagents) and operators.

### Interfering Substances and Stability Study Including the Effect of Light Exposure

The aim of this study was to assess the effect of haemolysis, icteria and lipaemia on assay results. In addition, the stability of spiked samples was investigated in different storage conditions (−20° C., 4° C., 37° C. and light/dark)

Two pools of serum samples were prepared. One was spiked with 500 ng/ml CLF, the other with 100 ng/ml CLF. Intralipid was spiked at 1 mg/ml into the 500 ng/ml pool, whereas bilirubin and haemoglobin were spiked at 1 mg/ml into the 100 ng/ml serum pool. In order to get the interfering substances haemoglobin and bilirubin into solution, 1 ml of 0.3M NaOH was added. The corresponding base pool was also prepared by adding 1 ml of 0.3M NaOH to 1 ml of the pool. The intralipid was added into the pool (no NaOH was added). These were tested at the beginning and end of the plate, in order to also look at any apparent assay drift).

Given the acceptability of the results at 1 mg/ml of each interferent, higher levels were examined; haemoglobin and bilirubin were also tested at concentrations of 4 and 4.5 mg/ml. However, the results obtained with both base and spiked pools were off scale (>1000 ng/ml), which is believed to be due to interference by the high level of NaOH present at these concentrations. The Intralipid was tested at a concentration of 230 mg/ml.

The stability study (−20° C., 4° C. and 37° C.) involved preparation of 3 serum pools spiked with 31.25 ng/ml, 125 ng/ml and 250 ng/ml CLF (aliquots of which were then stored at the 3 temperatures). Storage time was 48 hrs.

For the study of the effect of light, 3 pools of serum were spiked with CLF at 31.25, 125 and 500 ng/ml. One aliquot of each pool was put near a light source (fluorescent bulb 58 watts, at a distance of 85 cm). The “dark” sample was covered with aluminium foil. The samples were at RT (˜18-22° C.). Storage time was 48 hrs.

### Effect of Interfering Substances

The results are shown in the tables below.

Table 37 shows the concentrations in the base and test pools. Tables 37-39 are the results from the 1 mg/ml spikes.

TABLE 37

| | ng/ml | ng/ml |
|---|---|---|
| | beginning | end of |
| | of plate | plate |
| intralipid | 251 | 241 |
| base | | |
| intralipid | 239 | 246 |
| test | | |
| base | 84 | 87 |
| Hb | 83 | 83 |
| bilirubin | 90 | 86 |

Table 38 shows the % change in concentration due to the addition of the interfering factors.

TABLE 38

| | effect of | |
|---|---|---|
| | interference | effect of |
| | beginning | interference |
| | of plate | end of plate |
| intralipid | 0% | 0% |
| base | | |
| intralipid | −5% | 2% |
| test | | |
| base | 0% | 0% |
| Hb | −2% | −4% |
| bilirubin | 7% | −1% |

Table 39 shows the assay drift (% difference between results at the beginning and end of the plate.

TABLE 39

| | % drift |
|---|---|
| intralipid | |
| base | −4% |
| intralipid | |
| test | 3% |
| base | 3% |
| Hb | 1% |
| bilirubin | 4% |

The results from the high dose spikes are shown in Table 40.

TABLE 40

| | | % |
|---|---|---|
| | ng/ml | change |
| intralipid | | |
| base | 93 | |
| intralipid | | |
| test | 91 | −2% |
| base | off | |
| | scale | |
| Hb | off | |
| | scale | |
| bilirubin | off | |
| | scale | |

Table 41 shows the data from the −20° C. and 4° C., 48 hour storage. Table 42 shows the data from the −20° C. and 37° C., 48 hour storage.

TABLE 41

| | | | % |
|---|---|---|---|
| | | | difference |
| | Observed. | observed. | (4° C. from |
| Expected | −20° C. | 4° C. | −20° C.) |
| 31.25 | 37 | 34 | −8% |
| 125 | 110 | 107 | −2% |
| 250 | 240 | 211 | −12% |

TABLE 42

| | | | % difference |
|---|---|---|---|
| | observed. | observed. | (37° C. from |
| Expected | −20° C. | 37° C. | −20° C.) |
| 31.25 | 37 | 20 | −45% |
| 125 | 110 | 88 | −20% |
| 250 | 240 | >off scale | |

Table 43 shows the effect of light exposure after 48 hours at Room Temperature.

TABLE 43

| | | | % difference |
|---|---|---|---|
| | observed. | observed. | (light from |
| Expected | dark | Light | dark) |
| 31.25 | 37 | 37 | 0% |
| 125 | 143 | 107 | −25% |
| 500 | 495 | 423 | −15% |

The results presented suggest that the CLF assay is not unduly affected by haemolysis, icteria and lipaemia at interferent levels of 1 mg/ml. Evaluation of higher levels demonstrated excellent robustness to lipaemia (230 mg/ml), but no conclusions could be drawn on higher levels of haemolysis or icteria due to interference from the sodium hydroxide used to solubilise the interferents.

The data on the stability suggest that spiked samples can be stored for 48 hrs at 4° C. but should avoid higher temperatures, for example, 37° C.

In addition the results suggest that spiked CLF is unstable if exposed to light over a long period, with up to 25% of CLF immunoreactivity being lost after 48 hrs.

### Interfering Substances and Stability Study Including the Effect of Light Exposure

The aim of this study was to assess the effect of haemolysis, icteria and lipaemia on assay results. In addition, the stability of spiked samples was investigated in different storage conditions (−20° C., 4° C., 37° C. and light/dark).

Two pools of serum samples were prepared. One was spiked with 500 ng/ml CLF, the other with 100 ng/ml CLF. Intralipid was spiked at 1 mg/ml into the 500 ng/ml pool, whereas bilirubin and haemoglobin were spiked at 1 mg/ml into the 100 ng/ml serum pool. In order to get the interfering substances haemoglobin and bilirubin into solution, 1 ml of 0.3M NaOH was added. The corresponding base pool was also prepared by adding 1 ml of 0.3M NaOH to 1 ml of the pool. The intralipid was added into the pool (no NaOH was added). These were tested at the beginning and end of the plate, in order to also look at any apparent assay drift).

Given the acceptability of the results at 1 mg/ml of each interferent, higher levels were examined; haemoglobin and bilirubin were also tested at concentrations of 4 and 4.5 mg/ml. However, the results obtained with both base and spiked pools were off scale (>1000 ng/ml), which is believed to be due to interference by the high level of NaOH present at these concentrations. The Intralipid was tested at a concentration of 230 mg/ml.

The stability study (−20° C., 4° C. and 37° C.) involved preparation of 3 serum pools spiked with 31.25 ng/ml, 125 ng/ml and 250 ng/ml CLF (aliquots of which were then stored at the 3 temperatures). Storage time was 48 hrs.

For the study of the effect of light, 3 pools of serum were spiked with CLF at 31.25, 125 and 500 ng/ml. One aliquot of each pool was put near a light source (fluorescent bulb 58 watts, at a distance of 85 cm). The “dark” sample was covered with aluminium foil. The samples were at RT (˜18-22° C.). Storage time was 48 hrs.

### Effect of Interfering Substances

The results are shown in the tables 44-47 below.

Table 44 shows the concentrations in the base and test pools. Tables 1-3 are the results from the 1 mg/ml spikes.

TABLE 44

| | ng/ml | ng/ml |
|---|---|---|
| | beginning of | end of |
| | plate | plate |
| intralipid | | |
| base | 251 | 241 |
| intralipid | | |
| test | 239 | 246 |
| base | 84 | 87 |
| Hb | 83 | 83 |
| bilirubin | 90 | 86 |

Table 45 shows the % change in concentration due to the addition of the interfering factors.

TABLE 45

| | effect of | |
|---|---|---|
| | interference | effect of |
| | beginning of | interference |
| | plate | end of plate |
| intralipid | | |
| base | 0% | 0% |
| intralipid | | |
| test | −5% | 2% |
| base | 0% | 0% |
| Hb | −2% | −4% |
| bilirubin | 7% | −1% |

Table 46 shows the assay drift (% difference between results at the beginning and end of the plate.

TABLE 46

| | % drift |
|---|---|
| intralipid | |
| base | −4% |
| intralipid | |
| test | 3% |
| base | 3% |
| Hb | 1% |
| bilirubin | 4% |

The results from the high dose spikes are shown in Table 47.

TABLE 47

| | | % |
|---|---|---|
| | ng/ml | change |
| intralipid | | |
| base | 93 | |
| intralipid | | |
| test | 91 | −2% |
| base | off | |
| | scale | |
| Hb | off | |
| | scale | |
| bilirubin | off | |
| | scale | |

Table 48 shows the data from the −20° C. and 4° C., 48 hour storage. Table 49 shows the data from the −20° C. and 37° C., 48 hour storage.

TABLE 48

| | | | % |
|---|---|---|---|
| | | | difference |
| | observed. | observed. | (4° C. from |
| Expected | −20° C. | 4° C. | −20° C.) |
| 31.25 | 37 | 34 | −8% |
| 125 | 110 | 107 | −2% |
| 250 | 240 | 211 | −12% |

TABLE 49

| | | | % difference |
|---|---|---|---|
| | observed. | observed. | (37° C. from |
| Expected | −20° C. | 37° C. | −20° C.) |
| 31.25 | 37 | 20 | −45% |
| 125 | 110 | 88 | −20% |
| 250 | 240 | >off scale | |

Table 50 shows the effect of light exposure after 48 hours at Room Temperature.

TABLE 50

| | | | % difference |
|---|---|---|---|
| | observed. | observed. | (light from |
| Expected | dark | Light | dark) |
| 31.25 | 37 | 37 | 0% |
| 125 | 143 | 107 | −25% |
| 500 | 495 | 423 | −15% |

The results presented suggest that the CLF assay is not unduly affected by haemolysis, icteria and lipaemia at interferent levels of 1 mg/ml. Evaluation of higher levels demonstrated excellent robustness to lipaemia (230 mg/ml), but no conclusions could be drawn on higher levels of haemolysis or icteria due to interference from the sodium hydroxide used to solubilise the interferents.

The data on the stability suggest that spiked samples can be stored for 48 hrs at 4° C. but should avoid higher temperatures, for example, 37° C.

In addition the results suggest that spiked CLF is unstable if exposed to light over a long period, with up to 25% of CLF immunoreactivity being lost after 48 hrs.

### Assessment of Sample Drift and Stability of Spiked Serum Samples

Sample drift was assessed by using two sera spiked with CLF to 4 levels (62.5, 125, 250 and 500 ng/ml). Each of the samples were added to streptavidin coated wells (in duplicate) soon after the addition of the standards (time 0, in columns 5&6)); after 5 minutes the samples were added to columns 7&8; “5 minute” time point” 5 minutes later in columns 9&10 (“10 minute time point” and 5 minutes later in columns 11&12 (“15 minute time point”). The samples used were prepared 12 days prior to the experiment and were kept at 2-8° C. In addition, some other serum samples that were spiked with CLF at the same time (12 days prior to the assay) were also measured in order to determine the stability of CLF (note: The data obtained are the results of 2 variables (instability of CLF and the recovery) and thus represent a “worst case scenario”.

### Sample Drift

The data are sumamrised in table 51. None of the samples showed any significant drift over the 15 minute sample addition time.

TABLE 51

| Min | | 5 | 10 | 15 | 20 | mean | SD | % CV |
|---|---|---|---|---|---|---|---|---|
| #5885 | 62.5 | 42 | 49 | 47 | 45 | 46 | 2.7 | 6% |
| #5884 | 62.5 | 44 | 46 | 44 | 43 | 44 | 1.1 | 3% |
| #5885 | 125 | 82 | 82 | 88 | 83 | 84 | 2.9 | 3% |
| #5884 | 125 | 77 | 77 | 88 | 77 | 80 | 5.2 | 6% |
| #5885 | 250 | 173 | 185 | 187 | 163 | 177 | 11.5 | 7% |
| #5884 | 250 | 149 | 141 | 172 | 151 | 153 | 13.3 | 9% |
| #5885 | 500 | 383 | 397 | 381 | 353 | 379 | 18.3 | 5% |
| #5884 | 500 | 329 | 330 | 356 | 344 | 340 | 12.9 | 4% |

The level of instability (after 12 day storage at 2-8° C.) was calculated by comparing the results obtained with the expected results. The data are shown in the Table 52 below.

TABLE 52
12 day storage at 2-8° C.

| | Expected | observed | Difference |
|---|---|---|---|
| 5885 | 62.5 | 46 | −27% |
| 5884 | 62.5 | 44 | −29% |
| 5885 | 125 | 84 | −33% |
| 5884 | 125 | 80 | −36% |
| 5885 | 250 | 177 | −29% |
| 5884 | 250 | 153 | −39% |
| 5885 | 500 | 379 | −24% |
| 5884 | 500 | 340 | −32% |
| 5876 | 1000 | 756 | −24% |
| 5883 | 1000 | 903 | −10% |
| 5885 | 1000 | 804 | −20% |
| 5881 | 1000 | 820 | −18% |
| 5883 | 500 | 375 | −25% |
| pool1 | 125 | 76 | −39% |
| pool2 | 31.25 | 27 | −14% |
| pool3 | 500 | 365 | −27% |
| mean | | | −27% |

There was no significant drift, attributable to a 15 minute delay in sample addition. A significant reduction in measured CLF (mean 27%) occurs when samples are kept at 2-8° C. for 12 days. It is proposed that samples containing CLF should be kept frozen.

### Assay Drift and Recovery of Spiked CLF

Drift was examined by running samples at the beginning and end of the plate used to carry out the HPLC sample evaluation (see “Evaluation of HPLC samples”). Recovery was done by spiking CLF in two different human serum samples. One set (labelled as QC1 to QC3) was also used for inter-assay precision assessment (see separate paper). The concentrations of CLF spiked were 31.25, 125 and 500 ng/ml. This was prepared the day prior to the assay. The other spiking experiment was performed a week prior to the assay (plasma #5885 was spiked with 62.5, 125 and 500 ng/ml. These samples (and the QCs) were kept at 2-8° C. Freshly made standards were prepared by spiking CLF into buffer (concentration range of 0 to 1000 ng/ml).

TABLE 53
Recovery following CLF spiking

| | expected | observed | % recovery |
|---|---|---|---|
| QC2 | 125 | 119 | 95% |
| QC1 | 31.25 | 37 | 120% |
| QC3 | 500 | 438 | 88% |
| #5885 #1 | 62.5 | 63 | 101% |
| #5885 #2 | 125 | 119 | 95% |
| #5885 #3 | 500 | 485 | 97% |
| Mean | | | 99% |

The mean assay drift (difference in concentrations at the end of the plate from those at the beginning of the plate) was −4% (see Table 53 and FIG. 21).

The mean recovery after spiking of CLF into 2 sera was 99%.

Stability of Buffer-Based CLF Standards after 4 Weeks Storage at 2-8° C.

Standards were prepared by spiking CLF into buffer (6 standards having a concentration span of 0 to 1000 ng/ml). The standards were stored at 2-8° C. for 4 weeks. They were then taken out of the fridge and assayed alongside standards that were freshly made.

Analysis of the data is shown in the tables 54 & 55, and FIGS. 22 & 23.

TABLE 54
Concentration vs mean OD

| | | 4-week |
|---|---|---|
| | fresh | old stds |
| 0 | 4.164 | 4.145 |
| 16 | 2.745 | 3.219 |
| 31 | 2.265 | 2.699 |
| 63 | 1.653 | 2.133 |
| 125 | 0.884 | 1.577 |
| 250 | 0.661 | 0.953 |
| 500 | 0.419 | 0.528 |
| 1000 | 0.235 | 0.317 |

The data in table 54 are shown graphically in FIG. 22.

Table 55 shows the percent change in CLF immunoreactivity after storage of the standards for 4 weeks at 2-8° C. FIG. 2 shows a regression between the concentration of the fresh standards vs the stored standards.

TABLE 55
Percent change in the CLF immunoreactivity of the stored samples

| Fresh | 4-week | |
|---|---|---|
| stds | stds | % |
| (ng/ml) | (ng/ml) | change |
| 16 | 5 | −67% |
| 31 | 17 | −44% |
| 63 | 38 | −40% |
| 125 | 72 | −42% |
| 250 | 162 | −35% |
| 500 | 359 | −28% |
| 1000 | 685 | −31% |

Storage of the buffer-based CLF standards for 4 weeks at 2-8° C. resulted in a 30-40% decrease in immunoreactivity. It is clear that this degree of instability is unacceptable if there is a requirement for liquid standards. Options that need to be considered are (a) provision of freeze-dried standards, (b) investigation of alternative standard matrices and (c) provision of a concentrate which can be diluted prior to assay.

Conclusions from Feasibility Assay Performance Verification

The experimental programme successfully yielded a highly effective prototype ELISA for the quantification of CLF in human serum and plasma samples.

Areas requiring further attention are related to the stability of CLF—both with respect to storage conditions and shelf-life, and to correct handling of CLF-containing samples.

### Evaluation of HPLC Samples

### Sample Set 1

The first set of samples was assayed in duplicate, and gave the following results listed in table 56.

TABLE 56

| | Measured CLF (ng/ml) | | |
|---|---|---|---|
| Sample | Rep. 1 | Rep. 2 | Mean |
| LTD0 | 2.4 | 5.2 | 3.8 |
| LTD 1 | 6.5 | 9.3 | 7.9 |
| LTD 2.5 | 10.7 | 11.1 | 10.9 |
| LTD 5 | 13.0 | 15.5 | 14.3 |
| LTD 10 | 18.3 | 22.3 | 20.3 |
| LTD 25 | 46.8 | 44.9 | 45.8 |
| LTD 50 | 77.7 | 78.7 | 78.2 |
| LTD 100 | 139.7 | 150.4 | 145.1 |
| #2.5.1 T0 | 253.5 | 246.0 | 249.7 |
| #2.5.1 T30 4° C. | 252.8 | 260.4 | 256.6 |
| #2.5.1 T30 RT | 244.5 | 231.7 | 238.1 |
| #2.5.1 T120 | 239.4 | 265.4 | 252.4 |
| 4° C. | | | |
| #2.5.1 T120 RT | 255.8 | 215.4 | 235.6 |
| #2.5.1 24 h 4° C. | 232.1 | 224.0 | 228.1 |
| #2.5.1 T24 h RT | 221.9 | 215.0 | 218.5 |
| #2.5.2 T0 | 186.8 | 188.7 | 187.8 |
| #2.5.2 T30 4° C. | 209.2 | 204.8 | 207.0 |
| #2.5.2 T30 RT | 184.1 | 204.7 | 194.4 |
| #2.5.2 T120 | 220.4 | 192.0 | 206.2 |
| 4° C. | | | |
| #2.5.2 T120 | 169.8 | 208.4 | 189.1 |
| RT | | | |
| #2.5.2 T24 h | 177.4 | 181.2 | 179.3 |
| 4° C. | | | |
| #2.5.2 T24 h | 159.7 | 164.8 | 162.3 |
| RT | | | |
| #2.4 T0 | 303.5 | 320.0 | 311.8 |
| #2.4 T30 | 265.6 | 290.2 | 277.9 |
| #2.4 T120 | 315.8 | 234.7 | 275.3 |
| #2.4 T24 h | 265.5 | 292.2 | 278.8 |

The samples provided (LTD) included 8 “standards” (no details of their preparation were given, apart from the concentration which ranged from 0 to 100 ng/ml) and samples which were presumed to be stability samples (no details of their preparation were given). The stability work appeared to have been undertaken on 3 individual donor samples. Note, that 2 of the donor samples were heavily haemolysed.

TABLE 57
LTD standards

| LTD quoted | Elisa |
|---|---|
| values, | values, |
| ng/ml | ng/ml |
| 0 | 3.8 |
| 1 | 7.9 |
| 2.5 | 11 |
| 5 | 14 |
| 10 | 20 |
| 25 | 46 |
| 50 | 78 |
| 100 | 145 |

Regression of LTD quoted values and observed values (obtained with the CLF Elisa) is shown in FIG. 23.

### Stability (LTD Samples)

TABLE 58

| | percent |
|---|---|
| | loss of |
| | CLF, |
| | PLASMA |
| | 2.5.1 |
| T0 | 0% |
| T30, 4° C. | 3% |
| T30 RT | −5% |
| T120 4° C. | 1% |
| T120 RT | −6% |
| T24 h 4° C. | −9% |
| T24 h RT | −13% |

TABLE 59

| | percent loss of |
|---|---|
| | CLF, PLASMA |
| | 2.5.2 |
| T0 | 0% |
| T30, 4° C. | 10% |
| T30 RT | 3% |
| T120 4° C. | 10% |
| T120 RT | 1% |
| T24 h 4° C. | −5% |
| T24 h RT | −14% |

TABLE 60

| | percent |
|---|---|
| | loss of |
| | CLF, |
| | PLASMA |
| | 2.4 |
| T0 | 0% |
| T30 | −11% |
| T120 | −12% |
| T24 h | −11% |

There is an excellent correlation between the LTD quoted values (of the standards sent), but the slope (1.4) indicates a 40% difference in values (between quoted and obtained). Please note that the assay requirement is for an assay range of 20 ng/ml (sensitivity requirement) to 1000 ng/ml, while the standard sent only ranged from 0 to 100 ng/ml (5 of the 8 samples had quoted concentrations which were below the required sensitivity of 20 ng/ml).

Although no details were given on the stability samples, it appears that 4° C. storage is slightly better than RT (˜18-22° C.) storage. The percent loss at 24 hours was approximately 10% (see Tables 58-59).

### Sample Set 2

The second set of samples was assayed in duplicate, and gave the following results listed in Table 61.

TABLE 61

| | Measured CLF (ng/ml) | | |
|---|---|---|---|
| Sample | Rep. 1 | Rep. 2 | Mean |
| Human zero | −3.6 | −6.3 | <5 |
| Human CAL 1 | 0.4 | 1.2 | <5 |
| Human CAL 2 | 2.2 | 2.0 | <5 |
| Human CAL 3 | 10.2 | 10.1 | 10 |
| Human CAL 4 | 15.2 | 17.5 | 16 |
| Human CAL5 | 26.8 | 40.3 | 34 |
| Human CAL 6 | 65.0 | 78.3 | 72 |
| Human CAL 7 | 116.2 | 126.2 | 121 |
| Human D0 | 108.4 | 117.2 | 113 |
| Human D1 | 127.5 | 145.5 | 136 |
| Human D3 | 155.3 | 153.7 | 154 |
| Human D28 | 190.9 | 179.9 | 185 |
| Rat zero | 4.0 | 4.5 | <5 |
| Rat CAL 1 | 6.2 | 2.5 | <5 |
| Rat CAL 2 | 22.7 | 22.2 | 22 |
| Rat CAL 3 | 55.0 | 52.2 | 54 |
| Rat CAL 4 | 163.6 | 185.2 | 174 |
| Rat CAL 5 | 381.0 | 311.2 | 346 |
| Rat CAL 6 | 712.4 | 730.5 | 721 |
| Rat CAL 7 | 1272.1 | 1464.7 | 1368 |

Comparison of Standards Prepared from Aqueous Ethanol and Methanol Stock Solutions

Because of discrepancies between the expected and observed CLF concentrations for the HPLC-used standards tested in “Evaluation of HPLC samples” it was hypothesised that the discrepancy might be due to differences in the method of stock solution preparation employed (aqueous ethanol; see Appendix D) or methanol, and/or the repeated use of the same stock solution.

Therefore, a series of experiments were carried out to assess the impact of stock solution identity and age on measured CLF levels, comparing:
Freshly-prepared methanol stock solution
Freshly-prepared aqueous ethanol stock solution
Aged (3 days-4 weeks) aqueous ethanol stock solution

For each of the three stock preparations a set of standards, ranging from 0 to 1000 ng/ml were prepared in Buffer 24 (see Appendix B). These were then assayed together.

The standards prepared from the aged stock solution (3 week old) were used as the standard curve and the freshly prepared methanol and aqueous ethanol standards were treated as unknowns and read off the curve. Analysis of the data is shown in the FIG. 24.

The slopes produced by the freshly prepared stocks were 1.05 and 1.48, indicating a 5% difference (between expected and obtained concentrations) for the aqueous ethanol and a 48% difference for the methanol standards respectively.

The percentage difference observed for the methanol standards correlates with the 40% difference observed in “Evaluation of HPLC samples”. Together with percentage difference obtained with the aqueous ethanol standards the results suggest that the discrepancies were not due to the degradation of the aged aqueous ethanol stock but due to the differences in the method of preparing the stock solutions employed in the immunoassay and in the HPLC methodology.

APPENDIX A
RESULTS vs. FINAL PRODUCT PERFORMANCE
REQUIREMENT TARGETS

| | | Target Performance Characteristics (Key | Performance Obtained and | |
|---|---|---|---|---|
| # | Requirement | requirements are shown BOLD) | Reference | Comment |
| 1. | Target Markets | Clinical Research in DDIs | | |
| 2. | Market Segments | N/A | | |
| 3. | Assay Format | 96 microwell with 8 × 12 removable strips | A 96 microwell colorimetric ELISA | Requirement Met |
| | | manual colorimetric ELISA. | was developed | |
| 4. | Results output | Quantitative | A quantitative ELISA was developed | Requirement Met |
| 5. | Intended Use 1 | Research tool that can measure Cholyl- | | |
| | | Lysyl-Fluorescein (CLF) in (later projects | | |
| | | may require CE markings | | |
| 6. | Measurement units 1 | Ng/ml | | |
| 7. | Measurement Units 2 | N/A | | |
| 8. | Calibration reference | Antigen: Cholyl-Lysyl-Fluorescein (CLF), | | |
| | | CAS Nr 140616-46-2 (free acid form)The | | |
| | | molecular formula is: C51 H60 N3 Na3 O11S | | |
| | | and the MW is 992.07 | | |
| 9. | Normalisation | N/A | | |
| | parameter | | | |
| 10. | Body Fluids | Serum and EDTA/Heparin Plasma | The feasibility ELISA was used to | Requirement Met |
| | | | measure both serum and heparin | |
| | | | plasma samples; EDTA samples were | |
| | | | not evaluated | |
| 11. | Sample Size μl | ≦100 μl | 25 μl sample | Requirement Met |
| 12. | Sample dilution (range) | Dilution of samples is not desirable and will | Requirement 13 met | |
| | | not be adopted if requirement 13 can be met | | |
| 13. | Assay Range | 20-1000 ng/ml (no dilution) | An assay range of up to 1000 ng/ml | Requirement Met |
| | | | was developed | |
| 14. | Sensitivity (LDD) | <20 ng/ml | The actual sensitivity obtained was | Requirement Met |
| | | | 3.3 ng/ml. (Ref 4.2) | |
| 15. | Precision | <10% CV between assay | The inter-assay CV (across 6 assays | Requirement Met |
| | | | and 3 CLF concentrations) was <10% | |
| | | | (range was 4.5% to 9.4%). (Ref 4.1) | |
| 16. | Clinical sensitivity | N/A | | |
| 17. | Clinical specificity | N/A | | |
| 18. | Cross-reactants and | None to be tested (see limitations) | | |
| | highest concentration | | | |
| | that does not interfere. | | | |
| 19. | Clinical Categories to | N/A | | |
| | be tested (n) | | | |
| 20. | Limitations | Patients on fluorescein-containing | | |
| | | medication will be excluded from CLF assay. | | |
| 21. | Warnings | To be determined | | |
| 22. | Dilutional recovery | 100% +/− 10% | The mean dilution recovery was | Requirement not met but |
| | | | 112% over a concentration range of | capable of being met |
| | | | 15.6 to 1000 ng/ml (106% over a | with further development |
| | | | range of 31 to 100 ng/ml). (Ref 4.2) | |
| 23. | Comparator assay1 | A HPLC method may provide some | | |
| | and expected | comparative results but no specific | | |
| | agreement | studies/targets agreed | | |
| 24. | Comparator assay2 | N/A | | |
| | and expected | | | |
| | agreement | | | |
| 25. | Time to first result | <1 hour | A 40 minute assay protocol (1 step of | Requirement met. |
| | | | 30 minutes and a 10 minute substrate | |
| | | | incubation step) was developed | |
| 26. | Drift performance | <10% across the plate | The mean assay drift (difference in | Requirement met. |
| | | | concentrations at the end of the plate | |
| | | | from those at the beginning of the | |
| | | | plate) was −4%. (Ref 4.5) | |
| 27. | Other incubation time | N/A (analyte assumed to be stable) | | |
| | parameters | | | |
| 28. | Reagent stability and | Industry standard but research tool so 1 month | Storage of the buffer-based CLF | Requirement not met - |
| | shelf-lives | at 2-8 C. given programme length | standards for 4 weeks at 2-8 C. | further development |
| | (unopened/opened) | | resulted in a 30-40% decrease in | could provide suitable |
| | and transport needs | | immunoreactivity. (ref 4.6) | alternatives. |
| 29. | Test size | N/A | | |
| 30. | Protocol steps | As few as possible (1 step preferred) | A 1-step assay was developed | Requirement Met |
| 31. | QC fluids | Will be investigated. | No specific work carried out. | Further development |
| | | | | could evaluate QC |
| | | | | materials. |
| 32. | QC schemes | N/A | | |
| 33. | Interfering substances | Test standard bilirubin, haemoglobin and | The results presented suggest that | Requirement is Met |
| | | icteris at standard levels | the CLF assay is not unduly affected | |
| | | | by haemolysis, icteria and lipaemia. | |
| | | | The effect of spiking these potential | |
| | | | interferents at 1 mg/ml was minimal. | |
| | | | (ref 4.3) | |
| 34. | Incubation temperature | Room temperature not shaking preferred. | Room temperature non shaking | Requirement is Met |
| | | 37° C. shaking acceptable | protocol developed | |
| 35. | Reagent presentation | N/A | | |
| 36. | Calibrator Fluids | Buffer standards prepared by weight, liquid if | Liquid buffer and serum standards | Requirement is Met |
| | | possible | were prepared | |
| 37. | Calibrator IRP ref | N/A | | |
| 38. | Data reduction | Standard curve fit software | | |
| | methods | | | |
| 39. | Preservative name and | To be decided | | |
| | concentration | | | |
| 40. | Preferred buffer | Compatible with analyte and sample matrix | | |
| | composition | | | |
| 41. | Preferred antibody | N/A | | |
| | specificity/avidity/type | | | |
| 42. | Key reagent | N/A | | |
| | concentrations | | | |
| 43. | Sources of raw | To be agreed; no preference expressed on | | |
| | materials | polyclonal versus monoclonal antibodies. | | |
| 44. | Replicates | Duplicates | | |
| 45. | Presentation | N/A | | |
| 46. | Recovery after spiking | 100% +/− 10% | The spiking recovery data (mean % | Requirement is met |
| | | | recovery of 99%) suggest that the | |
| | | | assay is able to detect CLF added to | |
| | | | plasma over the assay range. The | |
| | | | mean recovery after spiking of CLF | |
| | | | into 2 sera was 99%. (Ref 4.5) | |
| 47. | Instrument | N/A manual assay only | | |
| | performance | | | |
| 48. | Sample stability | 1-2 months stability if possible, liquid | The data on the stability suggest that | Requirement not met; |
| | | preferred, freeze-dried acceptable | spiked samples can be stored for | further development or |
| | | | 48 hrs at 4 C. but should avoid higher | suggested storage |
| | | | temperatures e.g. 37 C. In addition the | alternatives would be |
| | | | results suggest that spiked CLF is | required. |
| | | | unstable if exposed to light over a | |
| | | | long period, with up to 25% of CLF | |
| | | | immunoreactivity being lost after | |
| | | | 48 hrs; (Ref 4.3) A significant | |
| | | | reduction in measured CLF (mean | |
| | | | 27%) occurs when samples are kept | |
| | | | at 2-8 C. for 12 days. It is proposed | |
| | | | that samples containing CLF should | |
| | | | be kept frozen. (Ref 4.4) | |
| 49. | Packaging/vials | N/A | | |

APPENDIX B
BUFFER FORMULATIONS

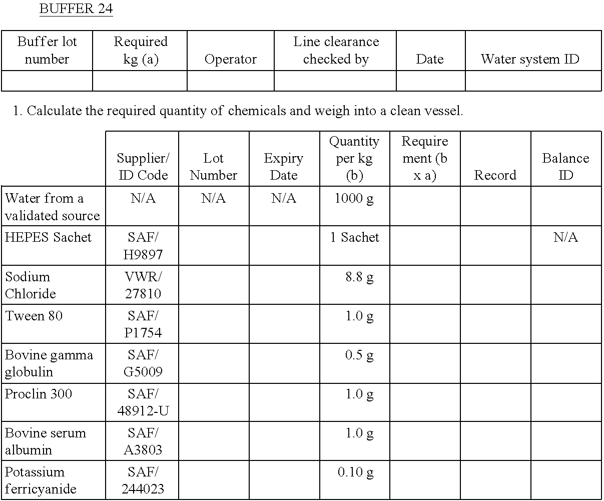

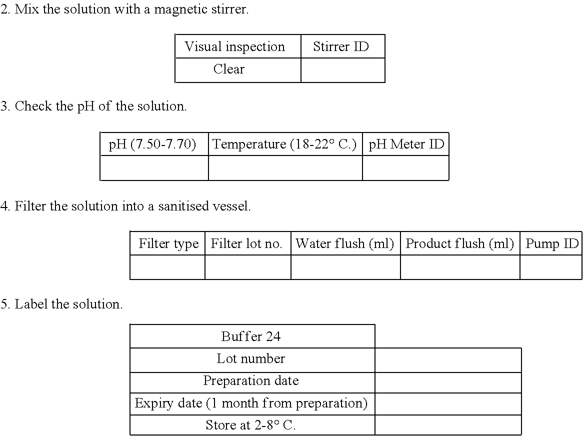

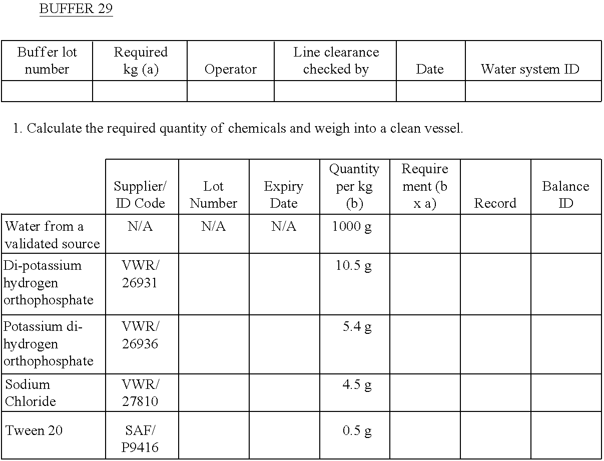

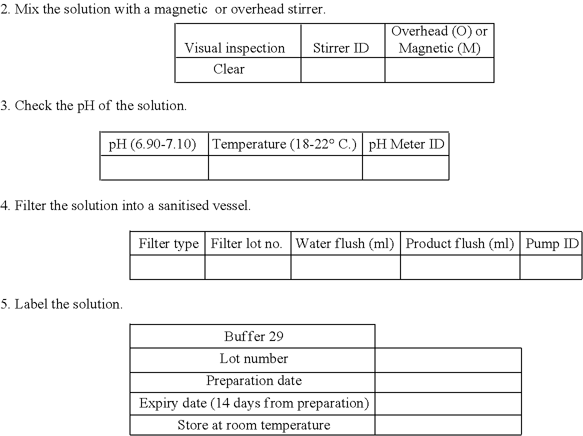

### APPENDIX C

### Anti-Fluorescein-HRP Conjugate Formulation (DV047/17-3)

Horseradish peroxidase (BBI Enzymes, HRP4; 25 mg/ml in 0.1M phosphate pH7.5) is activated with a 20:1 excess of SMCC (Thermo Fisher 22360; 17.5 mg/ml in DMSO) for 60 minutes at 20° C., the reaction quenched for 15 minutes at 20° C. with a volume of glycine solution (100 mg/ml in water) equivalent to 6.67% of the reaction volume, then purified on Sephadex G25M into 5 mM EDTA, 50 mM phosphate 150 mM NaCl pH 7.0 buffer (final HRP concentration 9.85 mg/ml).
In parallel, mouse monoclonal anti-fluorescein antibody (HyTest 5F3-2A3; 0.70 mg/ml in 0.1M phosphate pH7.5) is activated with a 20:1 excess of SATA (Thermo Fisher 26102; 1.0 mg/ml in DMSO) for 60 minutes at 20° C., the reaction deprotected for 15 minutes at 20° C. with a volume of 0.05M EDTA 2.5M Hydroxylamine pH 7.0 equivalent to 9.75% of the reaction volume then purified on Sephadex G25M into 5 mM EDTA, 50 mM phosphate 150 mM NaCl pH 7.0 buffer (final antibody concentration 0.28 mg/ml).
The activated HRP and activated antibody are incubated for 16 hours at 20° C. at a molar HRP:antibody ratio of 10:1, the reaction quenched with a volume of N-ethylmaleimide solution (1 mg/ml in water) equivalent to 0.43% of the reaction volume, then purified on Superdex 200PG into 50 mM phosphate 150 mM NaCl pH 6.7 buffer. The first major peak is collected (final antibody concentration 0.08 mg/ml, HRP: antibody incorporation 1.4:1)
1% (w/w) bovine serum albumin, 1% (w/v) Proclin 950 and (0.001% (w/v) potassium ferricyanide are added to the conjugate, which is filtered to 0.2 um and stored at 2-8° C.

### APPENDIX D

### Procedure Employed for Preparation of CLF Standards

To 1.0 mg of CLF is added sequentially 1000 ul of deionised water, 500 ul of ethanol and 30 ul of 50% aqueous sodium hydroxide solution, yielding a stock solution with a CLF concentration of 654 ug/ml.

This stock solution was stored at <−20° C. between each use, and thawed to room temperature before each use.

For the preparation of standards, this solution was diluted to 1000 ng/ml in Buffer 24 (see Appendix B), and thence by serial two-fold dilution into Buffer 24 to the required final concentrations.

### Example 4

### Dosage of Cholyl-L-Lysyl-Fluorescein (CLF) in Human Plasma by LC-MS/MS and Dosage of Three CLF Preperations by LC-UV (Formerly Appendix Dl

### Introduction

A high performance liquid chromatography tandem mass spectrometry assay was previously developed (Examples 1 and 2) for the analysis of Cholyl-Lysyl-L-Fluorescein (CLF) in biological samples. This methodology was used for quantification of CLF in rat and human plasma samples. Some differences were highlighted between quantification of CLF in human and rat plasma samples using HPLC-MS/MS method and the ELISA methodology. The differences obtained in human plasma samples spiked with CLF (from an in vitro study) were likely due to the method of preparation of working solutions. Thus, the following study was focused on 2 major points: (i) The comparison of solubility and stability of CLF in several solvents or mixtures by HPLC-UV-MS analysis, and (ii) the impact of working solution preparation methodology on analytical results (HPLC-MS/MS) in human plasma samples spiked with CLF.

### Material and Methods

### Reagents

Working solutions of CLF were prepared. Water, methanol and acetonitrile (LC-MS grade) were obtained from Carlo Erba (France). Formic acid and 5-([4,6-Dichlorotriazin-2-yl]amino)fluorescein hydrochloride as internal standard (IS) were purchased from Sigma Aldrich (France). Free-drug human plasma (pool of donors, mix gender, lithium heparin as anticoagulant), was purchased from Patricell (UK). Drug-free rat plasma was prepared from male Sprague Dawley rats.

### Materials

Analysis was performed on a HPLC-UV-MS/MS system consisting of an AmaZon SL ion trap mass spectrometer (Bruker) equipped with an ESI interface and an Ultimate 3000 device as HPLC system (Dionex) equipped with a thermostated autosampler, a thermostated column compartment and an UV detector. Data were acquired and processed using Hystar and its extensions QuantAnalysis and DataAnalysis (version 3.2, Bruker), respectively.

### Analytical Parameters

A high performance liquid chromatography tandem mass spectrometry assay has been developed (see Examples 1 and 2).

HPLC separation was operated with a mixture of water and acetonitrile containing 0.1% of formic acid and the reversed phase mode was selected. The injected volume of sample was 20 μl and the “full loop” mode was applied. The temperature of the analytical column was kept at 30° C. CLF, its metabolites and its IS were separated on a SunFire C₁₈ column (5 μm, 2.1×100 mm, Waters) by elution with a linear gradient of acetonitrile in water as described in Table 62.

The wavelength of UV detector was scaled at 254 nm. An ion trap mass spectrometer equipped with electrospray ionization (ESI) interface was operated in positive ionization mode. Detection was carried out in the following modes:
MS mode (full scan) in positive ESI for analysis of working and treatment solutions and metabolites identification,
MS/MS mode (MRM, multiple reaction monitoring) in positive ESI for qualitative analysis of CLF, its IS and its metabolites. Briefly, specific parent ions formed for CLF, its IS and its identified metabolites were selected for MS/MS fragmentations.

The analytical parameters are summarized in Table 62.

TABLE 62
Summary of analytical parameters

| | |
|---|---|
| Column | Acclaim 120 C₁₈ column (5 μm, 2.1 × 100 mm, |
| | Dionex) |
| Mobile phase A | 0.1% formic acid in 5/95 (acetonitrile/water; |
| | v/v) |
| Mobile phase B | 0.1% formic acid in acetonitrile |
| Linear gradient | From 20 to 100% of mobile phase B in 8 minutes |
| | Then, B = 100% during 2 min (8-10 minutes) |
| | Then, from 100 to 20% of mobile phase B in |
| | 2 minutes |
| | Total run time: 12 min |
| Flow rate | 200 μl/min |
| Injection volume | 20 μl in full loop mode |
| Column temperature | 30° C. |
| Wavelength | 254 nm |
| Polarity | Positive ESI |
| Capillary voltage | 4.5 kV |
| Dry gas flow | 8 l/min |
| Dry temperature | 200° C. |
| Nebulizer pressure | 15 psi |
| Desolvatation gas | Dinitrogen N₂ |
| CID gas | Helium |
| MS/MS mode | MRM mode |
| | CLF: m/z 926.5 > 537.3 and 926.4 > 390.0 |
| | IS: m/z 495.0 > 459.0 and 495.0 > 398.1 |
| | Metabolite: m/z 805.9 > 50-850 |
| MS mode | Full scan mode |
| | From 50 to 1500 amu |

The wavelength of UV detector was scaled at 254 nm. An ion trap mass spectrometer equipped with electrospray ionization (ESI) interface was operated in positive ionization mode. Detection was carried out in the following modes:

MS mode (full scan) in positive ESI for analysis of working and treatment solutions and metabolites identification, MS/MS mode (MRM, multiple reaction monitoring) in positive ESI for qualitative analysis of CLF, its IS and its metabolites. Briefly, specific parent ions formed for CLF, its IS and its identified metabolites were selected for MS/MS fragmentations.

### Note:

Negative ESI was assayed during the research of CLF metabolites. However, no additional peak was observed in the negative ionization mode. Thus, the positive ionization mode was conserved because of its better sensitivity.

### Quantification of CLF in Human Plasma Samples

Two working solutions of CLF were prepared. The first was prepared in 100% methanol for a final concentration of CLF at 109.6 μg/ml (SS CLF-MetOH) and the second was prepared in aqueous ethanol (water/ethanol/sodium hydroxide (50%), at 1000/500/30, v/v/v) for a final concentration of CLF at 71.6 μg/ml (SS CLF-EtOH).

Five human plasma samples, spiked with CLF from both working solutions, were prepared for HPLC-MS/MS analysis. These samples (named S1, S2, S3, S4 and S5) were thawed at room temperature, extemporaneously, and 100 μl of each were transferred in appropriate tubes.

The HPLC-MS/MS analysis was conducted with two calibration curves, constituted with 7 single points (non zero). The first one was prepared by serial dilutions in methanol of the working solution named SS CLF-MetOH. The second one was prepared by serial dilutions in methanol of the working solution named SS CLF-EtOH. Both working solutions were centrifuged 5 min at 3000 g before dilutions.

These dilutions of CLF in methanol allowed obtaining the 10× standard solutions used for standard sample preparation. Then, calibration curves were prepared by adding 10 μl of the concentrated standard solutions to 90 μl of free-drug human plasma. The final concentrations of calibrators were of 1000, 500, 250, 100, 25, 5 and 1 ng/ml.

An internal standard (IS), 5-([4,6-Dichlorotriazin-2-yl]amino)fluorescein hydrochloride, was added to each plasma sample (calibrators and test samples) in order to monitor the extraction. One aliquot of IS at 100 μg/ml in methanol was thawed and diluted at 2 μg/ml in acetonitrile. Then, 100 μl of acetonitrile spiked with IS were added to each human sample in order to precipitate proteins. Samples were vortex/mixed and centrifuged 5 min at 20000 g (4° C.). After centrifugation, the clear supernatant (around 160 μl) was transferred into 1.2 ml HPLC glass vials and sealed. Samples were placed into the refrigerated autosampler and 20 μl were injected into the HPLC-MS/MS system.

### Study of Solubility and Stability of CLF in Working and Treatment Solutions

Working solution used during this study were centrifuged 5 min at 3000 g and then diluted in a methanol/water mixture (50/50, v/v) in order to obtain three homogeneous solutions at 50 μg/ml. Then, 200 μl were transferred into 1.2 ml HPLC glass vials and sealed. Samples were placed into the refrigerated auto-sampler and 20 μl were injected into the HPLC-UV-MS system.

A blank sample was included (methanol/water, 50/50, v/v) to clearly distinguish interferences and impurities. The UV detection (scaled at 254 nm) allowed evaluating the solubility of CLF in both solvents by comparing CLF chromatographic peak areas. In case of signs of instability (additional chromatographic peaks), MS and then MS/MS (if needed) detections allowed to identify the impurities formed.

Note: The colour of both working solutions was very different. SS CLF-EtOH has a strong yellowish appearance while SS CLF-MetOH has very clear yellow appearance.

### Data Management

Analytical signals obtained by MS/MS detection were processed on HyStar 3.2 (and its extension: QuantAnalysis) software leading to integrate the chromatographic peaks of the test items. The response was expressed by the ratio of the chromatographic peak area of CLF with the chromatographic peak area of its internal standard. Chromatograms obtained by UV and simple MS detection were integrated manually using DataAnalysis Software. The contents of CLF were determined in 5 human plasma samples provided, according to the standard curve generated from both separate working solutions. Values were expressed in μg/ml. CLF metabolites identification was performed using DataAnalysis Software. The content of CLF was semi-quantified using data previously obtained. The UV response at 254 nm was used for semi-quantification. Raw data were processed using Excel (2007) and Graph Pad Prism 4.

### Results

### Chromatographic and Mass Spectrometric Properties of CLF

The selected mass spectrometric conditions led to the production of specific daughter ions from a specific [M+H]⁺ parent ion for CLF, as shown in FIGS. 25A and 25B. The parent ion formed was detected at m/z 926.4. Fragmentation of m/z 926.4 led to the formation of two major daughter ions detected at m/z 537.3 and m/z 390.0. The minor daughters ions observed (m/z 908.2, 519.3, 501.3, 483.3) corresponded to different loss of water from major ions formed. The hypothetical chemical structures of CLF specific daughter ions formed are illustrated in Table 7 (in Example 2 above). Details of mass spectrometric properties of IS are described in Example 1.

The selected HPLC-MS/MS conditions allowed a clear-cut for both separation and detection of the CLF and its IS, as shown by their retention times (see FIG. 5). Retention times were of 6.5 and 6.9 min for CLF and its IS, respectively.

### Study of Solubility and Stability of CLF in Working Solutions

### Chromatographic Profiles of Solutions

The UV chromatograms obtained at 254 nm are shown in FIG. 26.

In the working solution prepared in methanol (SS CLF-MetOH) and in a solution prepared in ammonium bicarbonate 0.1 M, one major chromatographic peak was observed. The retention time was of 6.5 min and corresponded to CLF.

In the working solution prepared in aqueous ethanol mixture (SS CLF-EtOH), two additional chromatographic peaks were observed at 5.7 and 9.7 min. Chromatographic peak areas are shown in Table 63.

TABLE 63
Chromatographic peak areas obtained with UV detection (254 nm)
[CLF] = 50 μg/ml
Peak Area

| | | SS CLF- | | Aqueous |
|---|---|---|---|---|
| | | MetOH | SS CLF-EtOH | ammonium |
| | Retention | working | working | bicarbonate |
| Compounds | time | solution | solution | solution (0.1M) |
| CLF | 6.5 min | 4395.1 | 470.9 | 5305.5* |
| Impurity 1 | 5.7 min | ND | 4389.8 | ND |
| Impurity 2 | 9.7 min | ND | 419.3 | ND |

ND: not detected
*Other batch of CLF

Peak area of CLF was around 10-fold lower in aqueous ethanol mixture than in methanol and aqueous ammonium bicarbonate 0.1 M. Thus, differences previously observed in both working solutions were likely due to instability of CLF.

### Identification of Impurities by Mass Spectrometry

Impurity 2 was not detected in mass spectrometry. The mass spectrum of compounds eluted at 5.7 and 6.5 min are shown in FIG. 27. Major ions detected for both compounds were similar. Thus, reanalysis of SS CLF-EtOH using the MS/MS detection mode (MRM of m/z 926.4) was performed. MS/MS spectrum obtained for both compounds are shown in FIG. 28. No difference between both compounds was highlighted using tandem mass spectrometry. Thus, both compounds observed here were likely two isomeric forms of CLF.

### Discussion

The fluorescein moiety of CLF is a tautomer. Tautomers are isomers (structural isomers) of organic compounds that readily interconvert by a chemical reaction called tautomerization. This reaction commonly results in the formal migration of a hydrogen atom or proton, accompanied by a switch of a single bond and adjacent double bond. Because of the rapid inter-conversion, tautomers are generally considered to be the same chemical compound. The tautomerization of CLF, and especially of the fluorescein moiety, is pH-dependant. The chemical reaction is shown in FIG. 6. Thus, parent and daughter ions detected in mass spectrometry, and formed for both isomeric forms, were similar as shown in Table 64.

TABLE 64
Chemical structure of main ions formed in mass spectrometry

| [M + H]⁺ | | |
|---|---|---|
| (m/z) | Hypothetical chemical structure (no charged form) | Ion type |
| 926.5 | 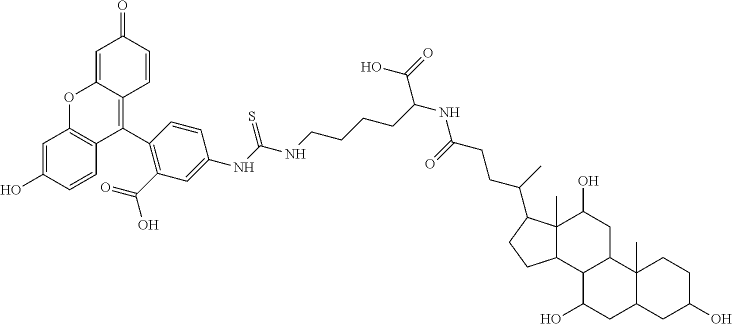 [SMILES: CC(CCC(=O)NC(CCCCNC(=S)Nc1ccc(-c2c3ccc(=O)cc-3oc3cc(O)ccc23)c(C(=O)O)c1)C(=O)O)C1CCC2C3C(O)CC4CC(O)CCC4(C)C3CC(O)C12C] | Parent ion |
| | or/and | |
| | 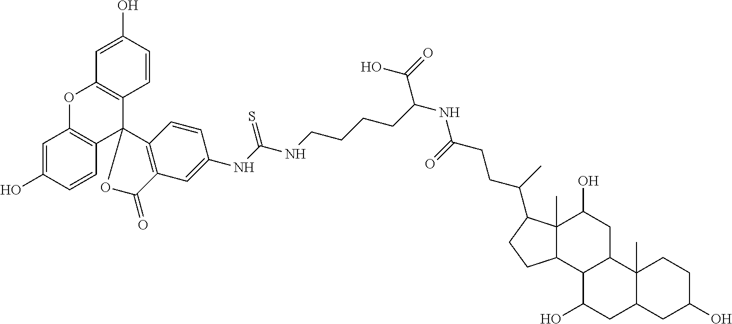 [SMILES: CC(CCC(=O)NC(CCCCNC(=S)Nc1ccc2c(c1)C(=O)OC21c2ccc(O)cc2Oc2cc(O)ccc21)C(=O)O)C1CCC2C3C(O)CC4CC(O)CCC4(C)C3CC(O)C12C] | |
| 537.3 | 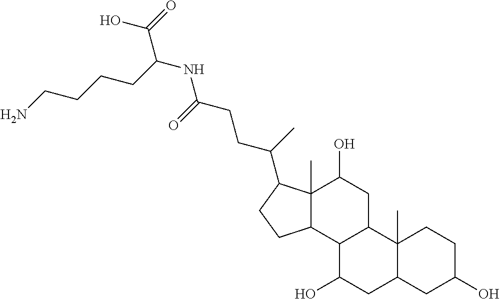 | Daughter ion of m/z 926.5 |
| 390.0 | 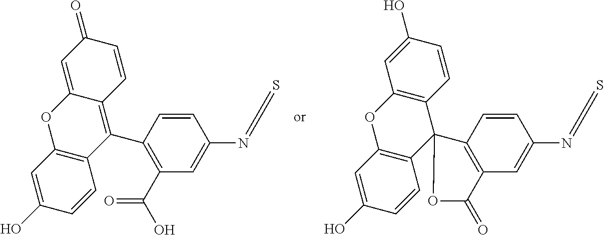 [SMILES: O=C(O)c1cc(N=C=S)ccc1-c1c2ccc(=O)cc-2oc2cc(O)ccc12 ; O=C1OC2(c3ccc(O)cc3Oc3cc(O)ccc32)c2ccc(N=C=S)cc21] | Daughter ion of m/z 926.5 |

CLF is in its native form when pH of the solution is below [pKa−2]. The addition of sodium hydroxide (strong base) has probably moved the steady state to the other tautomeric form (pH>pKa+2). However, in the ELISA assay, sodium hydroxide was diluted and buffered in the plasma and the pH of samples was decreased in order to obtain another steady state of CLF. The isomeric form of CLF was not detected in standard samples as shown in FIG. 30. The working solution was probably buffered in human plasma and during the sample extraction. This observation showed that the tautomerization of CLF is reversible and depends on the pH of the medium. Therefore in the ELISA assay, CLF was quantified in its native form. Fluorescence properties of both isomeric forms are probably also different. Indeed, quantum yield of fluorescence depends of the time-life of the excited state of the fluorophore (as shown by the color of working solutions). CLF was in its native form in the ammonium bicarbonate solution.

### Quantification of CLF in 5 Human Plasma Samples

### Standard Curves

Analysis of human plasma samples was conducted twice using each calibration curve. The isomeric form of CLF (tautomer), previously identified in the SS CLF-EtOH working solution, was not detected in standard samples as shown in FIG. 30. This working solution was probably buffered in human plasma and during the sample extraction. This observation showed that the tautomerization of CLF is reversible and is dependent on the pH of the medium.

Both calibration curves are shown in FIG. 31 and were processed with the 1/x weighed quadratic regression model. The r² values obtained were 0.9993 and 0.9998 for the first and the second calibration curves obtained from SS CLF-MetOH and SS CLF-EtOH working solutions, respectively.

### CLF Quantification in Human Samples

The five human samples, spiked with CLF at different concentration levels, were analyzed twice using each calibration curve. The CLF content in human samples are shown in Table 65. The isomeric form of CLF was not detected in human samples.

TABLE 65
Quantification of CLF ng/ml in human plasma samples.

| | From calibration curve | From calibration | | |
|---|---|---|---|---|
| | A | curve B | | |
| Sample | (SS CLF-MetOH) | (SS CLF-EtOH) | CV (%) | A-B (%) |
| S1 | 169.6 | 122.3 | 22.9 | 38.7 |
| S2 | 12.6 | 9.1 | 23.0 | 38.8 |
| S3 | 720.0 | 570.0 | 16.4 | 26.3 |
| S4 | 94.0 | 67.1 | 23.6 | 40.0 |
| S5 | 38.4 | 27.6 | 23.2 | 39.2 |

The coefficients of variation (CV, %) were calculated for each sample in order to evaluate differences between both quantifications. The CV % was of 16.4% for the highest concentration detected (sample S₃) and was into the range of 22.9-23.2 for the other samples.

The differences between both quantifications were also evaluated as follows:

$$A \text{-} B ( \% ) = \frac{Concentration A - Concentration B}{Concentration B} \times 100$$

The concentration of CLF in human plasma sample S3 was found 26.3% higher using the calibration curve A by comparing with the value obtained using the calibration curve B. In the other samples analyzed, the increase of CLF concentrations was into the range 38.7-40.0%.

### Conclusions

The following study has allowed us to highlight a number of properties of CLF. The first was the probable reversible tautomerization of the CLF when it is dissolved in a BASIC mixture. This isomeric transformation did not appear to have an impact on CLF quantification in plasma samples. However, the solvent used to dissolve CLF seemed to be important for the accuracy of the assay. The ammonium bicarbonate 0.1 M also seemed to be a suitable vehicle for preparing CLF for in vivo administration.

### REFERENCES

Hixson et al U.S. Pat. No. 4,264,514, 1981 Method and reagents for measuring the level of conjugated bile acids.
Mills, U.S. Pat. No. 6,030,841, 2000—Liver Function Test.
Dirk R. de Waart, Stephanie Ha\ddot{}usler, Maria L. H. Vlaming, Cindy Kunne, Emanuel Hanggi, Hans-Jurgen Gruss, Ronald P. J. Oude Elferink, and Bruno Stieger Hepatic Transport Mechanisms of Cholyl-L-Lysyl-Fluorescein JPET 334:78-86, 2010.
Mills C O, Milkiewicz P, Saraswat V, Elias E, Cholyl-lysyl-L-fluroscein and related lysyl fluorescein conjugated bile acid analogues, Yale J. Biol. Med. 70 (1997) 447-457.
Fayet M, Wahl P, The variation in the quantum yield and fluorescence decay of tyrosine as a function of pH, Biochim. et Biophys. Acta 229 (1971) 102-112
Principe d'ethique de l'experimentation animale, Directive n^{o} 86/609 CEE du 24 Nov. 1986, Décret n^{o} 87/848 du 19 Oct. 1987, Arrêté d'Application du 19 Avril 1988.
Roland F. Staack and Gerard Hopfgartner, New analytical strategies in studying drug metabolism, Anal. Bioanal. Chem., 2007, 388: 1365-1380.

1. A method for preparing a sample for analysis in an assay for Cholyl-L-Lysyl-Fluorescein (CLF), the method comprising contacting a sample comprising CLF with an alcohol under non-basic conditions.

2. A method according to claim 1, wherein the sample is a test sample obtained from a subject.

3. A method according to claim 1, wherein the sample is a blood sample, which is either serum or plasma.

4. A method according to claim 1, wherein the sample is a control sample, which is used to form a standard curve and against which an unknown test sample is measured.

5. A method according to claim 1, wherein the alcohol contacted with the sample comprises an aliphatic alcohol.

6. A method according to claim 5, wherein the alcohol comprises a C1-C5 alcohol.

7. A method according to claim 1, wherein the alcohol is selected from the group consisting of: methanol, ethanol and propanol.

8. A method according to claim 7, wherein the alcohol is methanol.

9. A method according to claim 1, wherein the concentration of alcohol following contacting with the sample is at least 30% volume per volume (v/v), 40% (v/v), 50% (v/v), 60% (v/v), or 70% (v/v).

10. A method according to claim 1, wherein the sample is contacted with an alcohol, wherein the pH is 7.0 or less.

11. A method according to any claim 1, wherein the sample is contacted with the alcohol in the absence of sodium hydroxide.

12. A Cholyl-L-Lysyl-Fluorescein (CLF) assay for determining the concentration of CLF in a sample, the method comprising:—(i) contacting a sample comprising CLF with an alcohol under non-basic conditions, and
(ii) quantifying the concentration of CLF in the sample using the method of claim 1, to thereby determine the concentration of the CLF.

13. (canceled)

14. An assay according to claim 12 or claim 13, wherein quantifying the concentration of CLF in the sample is performed using a detecting system selected from the group consisting of an immunoassay or HPLC.

15. An assay according to claim 14, wherein the immunoassay is an enzyme-linked immuno-specific assay (ELISA), and preferably a sandwich ELISA format.

16. An assay according to claim 14, wherein the method comprises using a competitive immunoassay for the detection of the CLF in the sample.

17. An assay according to claim 12, wherein the method further comprises measuring plasma levels of CLF using a fluorescein-specific monoclonal antibody.

18. An assay according to claim 12, wherein the method comprises determining the levels of CLF by measuring the signal generated by a coupled secondary antibody using enzyme-linked immuno-specific assay (ELISA) based on a known standard curve, which standard curve has been generated using the method of claim 1.

19. An assay according to claim 14, wherein the immunoassay comprises use of a biotin conjugated anti-FITC (fluorescein isothiocyanate) antibody, CLF (fluorescein (FITC)-conjugated bile salt) and a streptavidin-coated ELISA microtitre plate.

20. An assay according to claim 19, wherein FITG-biotin conjugates are captured on the ELISA plate.

21. An assay according to claim 20, wherein the fluorescein (FITC)-conjugated bile salt and FITC-biotin conjugate compete for binding to the anti-FITC-HRP antibody.

22. An assay according to claim 21, wherein unbound antibody is then removed, and the amount of remaining antibody is quantified colorimetrically, for example using tetramethylbenzidine (TMB) substrate.

23. An assay according to claim 14, wherein the immunoassay comprises the use of a blocking agent to inhibit binding between the fluorescein-conjugated bile salt and serum proteins.

24. An assay according to claim 23, wherein the blocking agent is selected from the group consisting of 8-anilino-i-naphthalene sulfonic acid (ANS) or deoxycholic acid.

25. A composition comprising Cholyl-L-Lysyl-Fluorescein (CLF) and an alcohol, wherein the composition does not comprise alkali.

26. A composition according to claim 25, wherein the alcohol is an aliphatic alcohol, and wherein the aliphatic alcohol is a C1-C5 alcohol selected from the group consisting of methanol, ethanol, and propanol.

27. A composition according to claim 25, wherein the composition is as defined in claim 10.

28. (canceled)

29. (canceled)

30. A method for determining drug-drug interactions (DDI) in a subject the method comprising the steps of (a) administering, into the bloodstream of a subject, Cholyl-L-Lysyl-Fluorescein (CLF);
(b) contacting a first blood sample, taken from the subject at time (T)=0 after the administration of one or more pharmacologically active compound(s), with an alcohol under non-basic conditions, and measuring the CLF concentration in the subject's plasma;
(c) contacting a second blood sample, taken from the subject at T=X, wherein X represents a unit of time after the administration of the one or more pharmacologically active compound(s), with an alcohol under non-basic conditions, and measuring the CLF concentration in the subject's plasma; and
(d) comparing the CLF concentration at T=X with that of T=0, whereby a difference in plasma levels of the CLF is determined, and the DDI in the subject is thereby evaluated.

31. An analyte or test composition comprising a physiological substrate conjugated to a signalling molecule, wherein the analyte or test composition is soluble in alcohol under non-basic conditions, for use in determining the in vivo serum or plasma levels of a composition whose metabolism is affected by the in vivo liver enzyme activity, wherein the liver enzyme activity corresponds to a drug-drug interaction, detoxification of a drug metabolite, or metabolism of the analyte.

32. An analyte or test composition according to claim 31, wherein the physiological substrate conjugated to a signalling molecule comprises Cholyl-L-Lysyl-Fluorescein (CLF).

33. An analyte or test composition according to claim 31, wherein the alcohol is as defined in claim 5 and wherein the composition is as defined in claim 10.

34. An analyte or test composition according to claim 31, wherein the alcohol is as defined in claim 5 and wherein the composition is as defined in claim 11

35. An analyte or test composition according to claim 31, wherein the composition is as defined in claim 10.

36. An analyte or test composition according to claim 31, wherein the composition is as defined in claim 11.

37. An assay according to claim 24, wherein the blocking agent comprises 8-anilirio-i-naphthalene sulfonic acid (ANS).

38. A composition according to claim 25, wherein the composition is as defined in claim 11.

39. A method for detection of cholestatic hepatoxicity in a subject, the method comprising the steps of (a) administering, into the bloodstream of a subject, Cholyl-L-Lysyl-Fluorescein (CLF);
(b) contacting a first blood sample, taken from the subject at time (T)=0 after the administration of one or more pharmacologically active compound(s), with an alcohol under non-basic conditions, and measuring the CLF concentration in the subject's plasma;
(c) contacting a second blood sample, taken from the subject at T=X, wherein X represents a unit of time after the administration of the one or more pharmacologically active compound(s), with an alcohol under non-basic conditions, and measuring the CLF concentration in the subject's plasma; and
(d) comparing the CLF concentration at T=X with that of T=0, whereby a difference in plasma levels of the CLF is determined, and cholestatic hepatoxicity in the subject is thereby evaluated.

40. The method of claim 30, where the subject is suffering from an unknown disease and wherein the method results in diagnosis of the disease and wherein the disease which is diagnosed is selected from a disease affecting an organ/tissue, wherein the organ/tissue is selected from the group consisting of liver, kidney, intestine, blood-brain barrier, blood-testicular barrier, and placenta.

41. The method of claim 39, where the subject is suffering from an unknown disease and wherein the method results in diagnosis of the disease and wherein the disease which is diagnosed is selected from a disease affecting an organ/tissue, wherein the organ/tissue is selected from the group consisting of liver, kidney, intestine, blood-brain barrier, blood-testicular barrier, and placenta.
